(19) United States
(12) Patent Application Publication
(10) Pub. No.: US 2005/0090505 A1
(43) Pub. Date: Apr. 28, 2005

(54) Methods of reducing risk of infection from pathogens

(76) Inventors: Michael R. Johnson, Chapel Hill, NC (US); Samuel E. Hopkins, Raleigh, NC (US)

Correspondence Address: Hutchison & Mason, PLLC, P.O. Box 31686, Raleigh, NC, 27612, US

(21) Appl. No.: 10/920,626

(22) Filed: Aug. 18, 2004

Related U.S. Application Data
(60) Provisional application No. 60/496,481, filed on Aug. 20, 2003.
(60) Provisional application No. 60/495,725, filed on Aug. 18, 2003.
(60) Provisional application No. 60/495,712, filed on Aug. 18, 2003.
(60) Provisional application No. 60/495,720, filed on Aug. 18, 2003.

(51) Int. Cl. A61K 31/4965; A61K 31/497

(52) U.S. Cl. 514/255.05; 514/255.06

(57) ABSTRACT

Prophylactic treatment methods are provided for protection of individuals and/or populations against infection from airborne pathogens. In particular, prophylactic treatment methods are provided comprising administering a sodium channel blocker or pharmaceutically acceptable salts thereof to one or more members of a population at risk of exposure to or already exposed to one or more airborne pathogens, either from natural sources or from intentional release of pathogens into the environment.

### CROSS REFERENCE TO RELATED APPLICATIONS

This application claims the benefit of U.S. Provisional Application Nos. 60/496,481, filed Aug. 20, 2003, 60/495,725, filed Aug. 19, 2003, 60/495,712, filed Aug. 19, 2003 and 60/495,720, filed Aug. 19, 2003, each of which is incorporated herein by reference in its entirety.

### BACKGROUND

1. Field of the Invention

The present invention relates to the use of sodium channel blockers for prophylactic, post-exposure prophylactic, preventive or therapeutic treatment against diseases or conditions caused by pathogens, particularly pathogens which may be used in bioterrorism.

2. Description of the Related Art

In recent years, a variety of research programs and biodefense measures have been put into place to deal with concerns about the use of biological agents in acts of terrorism. These measures are intended to address concerns regarding bioterrorism or the use of microorganisms or biological toxins to kill people, spread fear, and disrupt society. For example, the National Institute of Allergy and Infectious Diseases (NIAID) has developed a Strategic Plan for Biodefense Research which outlines plans for addressing research needs in the broad area of bioterrorism and emerging and reemerging infectious diseases. According to the plan, the deliberate exposure of the civilian population of the United States to Bacillus anthracis spores revealed a gap in the nation's overall preparedness against bioterrorism. Moreover, the report details that these attacks uncovered an unmet need for tests to rapidly diagnose, vaccines and immunotherapies to prevent, and drugs and biologics to cure disease caused by agents of bioterrorism.

Much of the focus of the various research efforts has been directed to studying the biology of the pathogens identified as potentially dangerous as bioterrorism agents, studying the host response against such agents, developing vaccines against infectious diseases, evaluating the therapeutics currently available and under investigation against such agents, and developing diagnostics to identify signs and symptoms of threatening agents. Such efforts are laudable but, given the large number of pathogens which have been identified as potentially available for bioterrorism, these efforts have not yet been able to provide satisfactory responses for all possible bioterrorism threats. Additionally, many of the pathogens identified as potentially dangerous as agents of bioterrorism do not provide adequate economic incentives for the development of therapeutic or preventive measures by industry. Moreover, even if preventive measures such as vaccines were available for each pathogen which may be used in bioterrorism, the cost of administering all such vaccines to the general population is prohibitive.

Until convenient and effective treatments are available against every bioterrorism threat, there exists a strong need for preventative, prophylactic or therapeutic treatments which can prevent or reduce the risk of infection from pathogenic agents.

### BRIEF SUMMARY

The present invention provides such methods of prophylactic treatment. In one embodiment, a prophylactic treatment method is provided comprising administering a prophylactically effective amount of a sodium channel blocker according to Formula I: 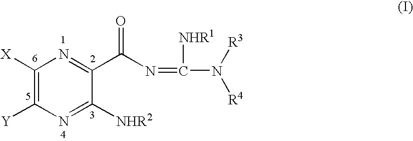
wherein
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 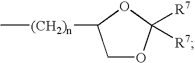
R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A): 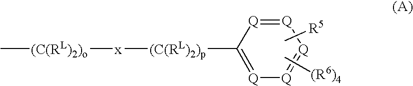
   wherein
each R^{L} is, independently, —R⁷, —(CH₂)ₙ—OR⁸, —O—(CH₂)ₘ—OR⁸, —(CH₂)ₙ—NR⁷R⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—O—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 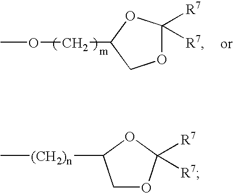
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
wherein each R⁵ is, independently,
Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ-(Z)_{g}-CAP, Link —(CH₂)ₙ(Z)_{g}-(CH₂)ₘ—CAP , Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}-CAP, Link —(CH₂)ₙNR¹³—(CH₂)ₘ(CHOR⁸)ₙCH₂NR¹³-(Z)_{g}-CAP, Link —(CH₂)ₘ-(Z)_{g}-(CH₂)ₘ—CAP, Link NH—C(═O)NH(CH₂)ₘ—CAP, Link —(CH₂)ₘ—C(═O)NR¹³—(CH₂)ₘ—C(═O)NR¹⁰R¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)ₘ-Het-(CH₂)ₘ—CAP;
wherein Link is, independently,
—O—, (CH₂)ₙ—, —O(CH₂)ₘ—, —NR¹³—C(═O)—NR¹³, —NR¹³C(═O)—(CH₂)ₘ—, —C(═O)NR¹³—(CH₂)ₘ, —(CH₂)ₙ-Z_{g}-(CH₂)ₙ, —S—, —SO—, —SO₂—, SO₂NR⁷—, SO₂NR¹⁰—, -Het-;
wherein each CAP is, independently, thiazolidinedione, oxazolidinedione, heteroaryl-C(═O)NR¹³R¹³, heteroaryl-CAP, —CN, —O—C(═S)NR¹³R¹³, -Z_{g}R¹³, —CR¹⁰(Z_{g}R¹³)(Z_{g}R¹³), —C(═O)OAr, —C(═O)NR¹³Ar, imidazoline, tetrazole, tetrazole amide, —SO₂NHR¹³, —SO₂NH—C(R¹³R¹³)-(Z)_{g}-R¹³, cyclic sugars and oligosaccharides, including cyclic amino sugars and oligosaccharides, 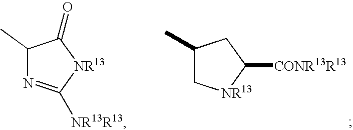
wherein Ar is, independently, phenyl; substituted phenyl, wherein said substituent is 1-3 groups selected, independently, from OH, OCH₃, NR¹³R¹³, Cl, F, CH₃; heteroaryl, e.g., pyridine, pyrazine, tinazine, furyl, furfuryl-, thienyl, tetrazole, thiazolidinedione and imidazoyl ( 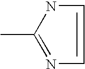
   ) and other heteroaromatic ring systems as defined below;
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
each R⁶ is, independently, —R⁷, —OR⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 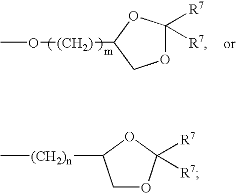
where when two R⁶ are —OR¹¹ and are located adjacent to each other on a phenyl ring, the alkyl moieties of the two R⁶ may be bonded together to form a methylenedioxy group;
with the proviso that when at least two —CH₂OR⁸ are located adjacent to each other, the R⁸ groups may be joined to form a cyclic mono- or di-substituted 1,3-dioxane or 1,3-dioxolane,
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 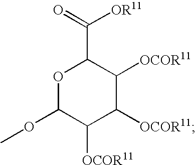
each R⁹ is, independently, —CO₂R¹³, —CON(R¹³)₂, —SO₂CH₂R¹³, or —C(═O)R¹³;
each R¹⁰ is, independently, —H, —SO₂CH₃, —CO₂R¹³, —C(═O)NR¹³R¹³,
—C(═O)R¹³, or —CH₂)ₘ—(CHOH)ₙ—CH₂OH;
each Z is, independently, CHOH, C(═O), —(CH₂)ₙ—CHNR¹³R¹³, C═NR¹³, or NR¹³;
each R¹¹ is, independently, lower alkyl;
each R¹² is independently, —SO₂CH₃, —CO₂R¹³, —C(═O)NR¹³R¹³, —C(═O)R¹³, or —CH₂—(CHOH)ₙ—CH₂OH;
each R¹³ is, independently, hydrogen, R⁷, R¹⁰, —(CH₂)ₘ—NR¹³R¹³, 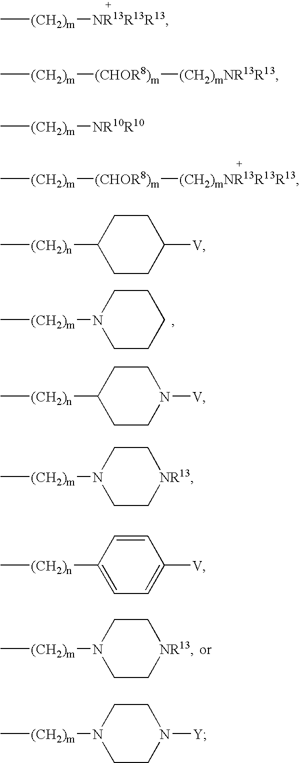
with the proviso that NR¹³R¹³ can be joined on itself to form a ring comprising one of the following: 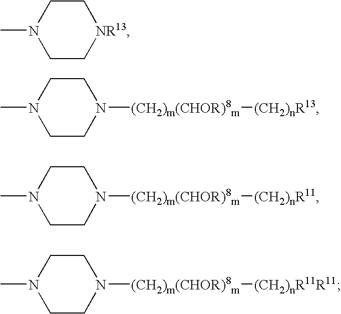
each Het is independently, —NR¹³, —S—, —SO—, or —SO₂—; —O—, —SO₂NR¹³—, —NHSO₂—, —NR¹³CO—, —CONR¹³—;
each g is, independently, an integer from 1 to 6;
each m is, independently, an integer from 1 to 7;
each n is, independently, an integer from 0 to 7;
each Q is, independently, C—R⁵, C—R⁶, or a nitrogen atom, wherein atom wherein at
most three Q in a ring are nitrogen atoms;
each V is, independently, —(CH₂)ₘ—NR⁷R¹⁰, —(CH₂)ₘ—NR⁷R⁷, —(CH₂)ₘ— 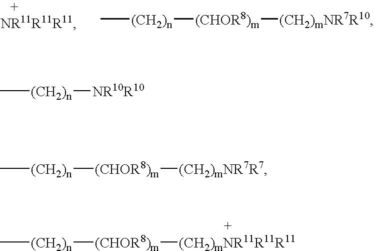
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane, or a pharmaceutically acceptable salt thereof to an individual in need of prophylactic treatment against infection from one or more airborne pathogens.

In another embodiment, a prophylactic treatment method is provided comprising administering a prophylactically effective amount of a sodium channel blocker according to Formula II: 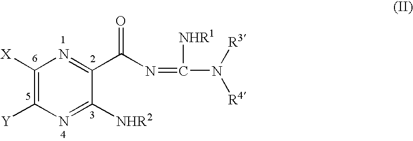
where
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 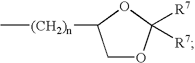
R^{3′} and R^{4′} are each, independently, hydrogen, a group represented by formula (A′), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R^{3′} and R^{4′} is a group represented by formula (A′):
   —(C(R^{L})₂)_{O}-x-(C(R^{L})₂)_{P}-CR^{5′}R^{6′}R^{6′} (A′)
   where
each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR₈)ₙ—CH₂OR⁸, —(CH₂)ₙ—CO₂R⁷, —O—(CH₂)ₘ—CO₂R⁷, —OSO₃H, —O-glucuronide, —O-glucose, 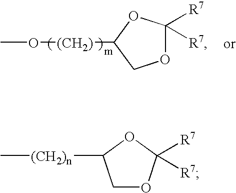
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
each R^{5′} is, independently, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 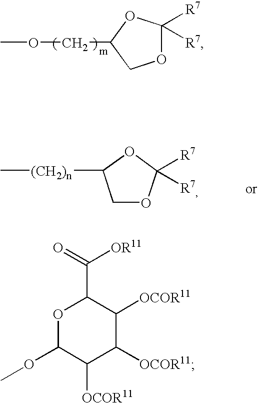
each R^{5′} is also, independently, —(CH₂)ₙ—NR¹²R¹², —O—(CH₂)ₘ—NR¹²R¹², —O—(CH₂)ₙ—NR¹²R¹², —O—(CH₂)ₘ-(Z)_{g}R¹², —(CH₂)ₙNR¹¹R¹¹, —O—(CH₂)ₘNR¹¹R¹¹, —(CH₂)ₙ—N^{⊕}—(R¹¹)₃, —O—(CH₂)ₘ—N^{⊕}—(R¹¹)₃, —(CH₂)ₙ-(Z)_{g}-(CH₂)ₘ—NR¹⁰R¹⁰, —O—(CH₂)ₘ-(Z)_{g}-(CH₂)ₘ—NR¹⁰R¹⁰, —(CH₂CH₂O)ₘ—CH₂CH₂NR¹²R¹², —O—(CH₂CH₂O)ₘ—CH₂CH₂NR¹²R¹², —(CH₂)ₙ—(C═O)NR¹²R¹², —O—(CH₂)ₘ—(C═O)NR¹²R¹², —O—(CH₂)ₘ—(CHOR⁸)ₘCH₂NR¹⁰-(Z)_{g}-R¹⁰, —(CH₂)ₙ—(CHOR⁸)ₘCH₂—NR¹⁰-(Z)_{g}-R¹⁰, —(CH₂)ₙNR¹⁰—O(CH₂)ₘ(CHOR⁸)ₙCH₂NR¹⁰-(Z)_{g}-R¹⁰, —O(CH₂)ₘ—NR¹⁰—(CH₂)ₘ—(CHOR⁸)ₙCH₂NR¹⁰-(Z)_{g}-R¹⁰, -(Het)-(CH₂)ₘ—OR⁸, -(Het)-(CH₂)ₘ—NR⁷R¹⁰, -(Het)-(CH₂)ₘ(CHOR⁸)(CHOR⁸)ₙ—CH₂OR⁸, -(Het)-(CH₂CH₂O)ₘ—R⁸, -(Het)-(CH₂CH₂O)ₘ—CH₂CH₂NR⁷R¹⁰, -(Het)-(CH₂)ₘ—C(═O)NR⁷R¹⁰, -(Het)-(CH₂)ₘ-(Z)_{g}-R⁷, -(Het)-(CH₂)ₘ—NR¹⁰—CH₂(CHOR⁸)(CHOR⁸)ₙ—CH₂OR⁸, -(Het)-(CH₂)ₘ—CO₂R⁷, -(Het)-(CH₂)ₘ—NR¹²R¹², -(Het)-(CH₂)ₙ—NR¹²R¹², -(Het)-(CH₂)ₘ-(Z)_{g}R¹², -(Het)-(CH₂)ₘNR¹¹R¹¹, -(Het)-(CH₂)ₘ—N^{⊕}—(R¹¹)₃, -(Het)-(CH₂)ₘ-(Z)_{g}-(CH₂)ₘ—NR¹⁰OR¹⁰, —(Het)-(CH₂CH₂O)ₘ—CH₂CH₂NR¹²R¹², -(Het)-(CH₂)ₘ—(C═O)NR¹²R², -(Het)-(CH₂)ₘ—(CHOR⁸)ₘCH₂NR¹⁰-(Z)_{g}-R¹⁰, -(Het)-(CH₂)ₘ—NR¹⁰—(CH₂)ₘ—(CHOR⁸)ₙCH₂NR¹⁰-(Z)_{g}-R¹⁰,
wherein when two —CH₂OR⁸ groups are located 1,2- or 1,3- with respect to each other the R⁸ groups may be joined to form a cyclic mono- or di-substituted 1,3-dioxane or 1,3-dioxolane,
—(CH₂)ₙ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—O—(CH₂)ₘ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—(CH₂)ₙ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane, or
—O—(CH₂)ₘ—NR¹⁰—CH₂(CHOR⁸)(CHOR⁸)ₙCH₂OR⁸, with the proviso that at least two —CH₂OR⁸ are located adjacent to each other and the R⁸ groups are joined to form a cyclic mono- or di-substituted 1,3-dioxane or 1,3-dioxolane;
wherein each R^{5′} is also, independently,
Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ-(Z)_{g}-CAP, Link —(CH₂)ₙ(Z)_{g}-(CH₂)ₘ—CAP, Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}-CAP, Link —(CH₂)ₙNR¹³—(CH₂)ₘ(CHOR⁸)ₙCH₂NR¹³-(Z)_{g}-CAP, Link —(CH₂)ₘ-(Z)_{g}-(CH₂)ₘ—CAP, Link NH—C(═O)—NH—(CH₂)ₘ—CAP, Link —(CH₂)ₘ—C(═O)NR¹³—(CH₂)ₘ—C(═O)NR¹⁰R¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)ₘ-Het-(CH₂)ₘ—CAP;
wherein Link is, independently, —O—, (CH₂)ₙ—, —O(CH₂)ₘ—, —NR¹³—C(═O)—NR¹³, —NR¹³—C(═O)—(CH₂)ₘ—, —C(═O)NR¹³—(CH₂)ₘ, —(CH₂)ₙ-Z_{g}-(CH₂)ₙ, —S—, —SO—, —SO₂—, SO₂NR⁷—, SO₂NR¹⁰—, -Het-;
wherein each CAP is, independently, thiazolidinedione, oxazolidinedione, heteroaryl-C(═O)NR¹³R¹³ , heteroaryl-CAP, —CN, —O—C(═S)NR¹³R¹³, -Z_{g}R¹³, —CR¹⁰(Z_{g}R¹³)(Z_{g}R¹³), —C(═O)OAr, —C(═O)NR¹³Ar, imidazoline, tetrazole, tetrazole amide, —SO₂NHR¹³, —SO₂NH—C(R¹³R¹³)-(Z)_{g}-R¹³, cyclic sugars and oligosaccharides, including cyclic amino sugars and oligosaccharides, 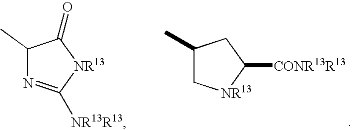
wherein Ar is, independently, phenyl; substituted phenyl, wherein said substituent is 1-3 groups selected, independently, from OH, OCH₃, NR¹³R¹³, Cl, F, CH₃; heteroaryl, e.g., pyridine, pyrazine, tinazine, furyl, furfuryl-, thienyl, tetrazole, thiazolidinedione and imidazoyl ( 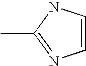
   ) and other heteroaromatic ring systems as defined below;
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
wherein when two —CH₂OR⁸ groups are located 1,2- or 1,3- with respect to each other the R⁸ groups may be joined to form a cyclic mono- or di-substituted 1,3-dioxane or 1,3-dioxolane,

each R^{6′} is, independently, —R^{5′}, —R⁷, —OR⁸, —N(R⁷)₂, —(CH₂)ₘ—OR⁸, —O—(CH₂)ₘ—OR⁸, —(CH₂)ₘ—NR⁷R¹⁰, —O—(CH₂)ₘ—NR⁷R¹⁰, —(CH₂)ₙ(CHOR⁸)(CHOR⁸)ₙ—CH₂OR⁸, —O(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—OC₂R⁷, —OSO₃H, —O-glucuronide, —O-glucose, 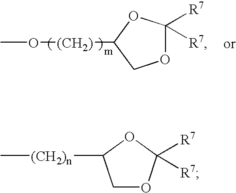
wherein when two —CH₂OR⁸ groups are located 1,2- or 1,3- with respect to each other the R⁸ groups may be joined to form a cyclic mono- or di-substituted 1,3-dioxane or 1,3-dioxolane;
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or

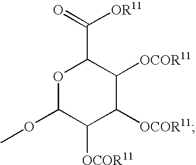

each R⁹ is, independently, —CO₂R¹³, —CON(R¹³)₂, —SO₂CH₂R¹³, or —C(═O)R¹³;
each R¹⁰ is, independently, —H, —SO₂CH₃, —CO₂R¹³R—C(═O)NR¹³R¹³, —C(═O)R¹³, or —(CH₂)ₘ—(CHOH)ₙ—CH₂OH;
each Z is, independently, CHOH, C(═O), —(CH₂)ₙ—, CHNR¹³R¹³, C═NR¹³, or NR¹³;
each R¹¹ is, independently, lower alkyl;
each R¹² is independently, —SO₂CH₃, —CO₂R¹³, —C(═O)NR¹³R¹³, —C(═O)R¹³, or —CH₂—(CHOH)ₙ—CH₂OH;
each R¹³ is, independently, hydrogen, R⁷, R¹⁰, —(CH₂)ₘ—NR¹³R¹³, 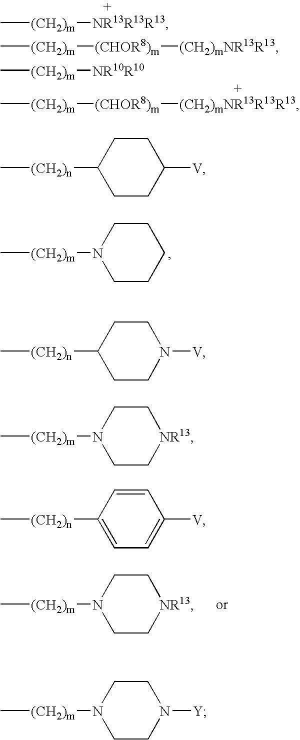
with the proviso that NR¹³R¹³ can be joined on itself to form a ring comprising one of the following: 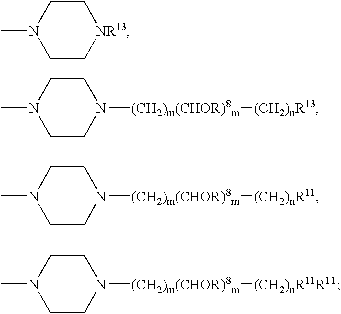
each Het is independently, —NR¹³, —S—, —SO—, or —SO₂—; —O—, —SO₂NR¹³—, —NHSO₂—, —NR¹³CO—, —CONR¹³—;
each g is, independently, an integer from 1 to 6;
each m is, independently, an integer from 1 to 7;
each n is, independently, an integer from 0 to 7;
each V is, independently, —(CH₂)ₘ—NR⁷R¹⁰, —(CH₂)ₘ—NR⁷R⁷, —(CH₂)ₘ— 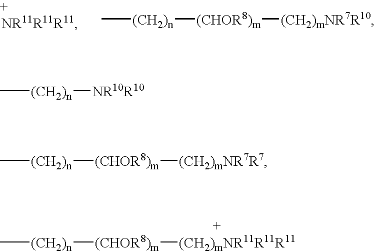
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane; or a pharmaceutically acceptable salt thereof to an individual in need of prophylactic treatment against infection from one or more airborne pathogens.

In another embodiment, a prophylactic treatment method is provided comprising administering a prophylactically effective amount of a sodium channel blocker according to Formula III: 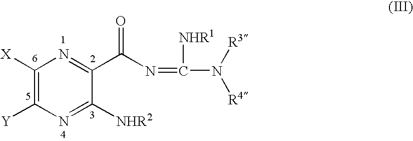
where
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 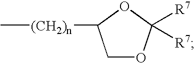
R^{3″} and R^{4″} are each, independently, hydrogen, a group represented by formula (A″), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R^{3′} and R^{4″} is a group represented by formula (A″): 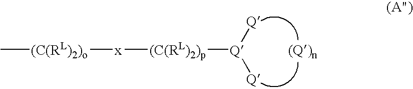
   where
each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 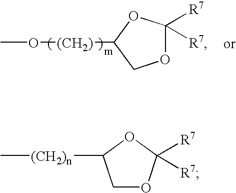
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰),
CHNR⁷R¹⁰, or represents a single bond;
each R^{5′} is, independently, independently, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 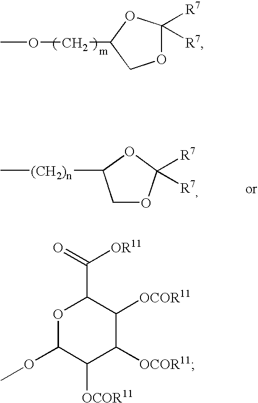
each R^{5′} is also, independently, —(CH₂)ₙ—NR¹²R¹², —O—(CH₂)ₘ—NR¹²R¹², —O—(CH₂)ₙ—NR¹²R¹², —O—(CH₂)ₘ-(Z)_{g}R¹², —(CH₂)ₙNR¹¹R¹¹, —O—(CH₂)ₘNR¹¹R¹¹, —(CH₂)ₙ—N^{⊕}—(R¹¹)₃, —O—(CH₂)ₘ—N^{⊕}—(R¹¹)₃, —(CH₂)ₙ-(Z)_{g}-(CH₂)ₘ—NR¹⁰OR¹⁰, —O—(CH₂)ₘ-(Z)_{g}-(CH₂)ₘ—NR¹⁰R¹⁰, —(CH₂CH₂O)ₘ—CH₂CH₂NR¹²R¹², —O—(CH₂CH₂O)ₘ—CH₂CH₂NR¹²R¹², —(CH₂)ₙ—(C═O)NR¹²R¹², —O—(CH₂)ₘ—(C═O)NR¹²R¹², —O—(CH₂)ₘ—(CHOR⁸)ₘCH₂NR¹⁰-(Z)_{g}-R¹⁰, —(CH₂)ₙ—(CHOR⁸)ₘCH₂—NR¹⁰-(Z)_{g}-R¹⁰, —(CH₂)ₙNR¹⁰—O(CH₂)ₘ(CHOR⁸)ₙCH₂NR¹⁰-(Z)_{g}-R¹⁰, —O(CH₂)ₘ—NR¹⁰—(CH₂)ₘ—(CHOR⁸)ₙCH₂NR¹⁰-(Z)_{g-R}¹⁰, -(Het)-(CH₂)ₘ—OR⁸, -(Het)-(CH₂)ₘ—NR⁷R¹⁰, -(Het)-(CH₂)ₘ(CHOR⁸)(CHOR⁸)ₙ—CH₂OR⁸, -(Het)-(CH₂CH₂O)ₘ—R⁸, -(Het)-(CH₂CH₂O)ₘ—CH₂CH₂NR⁷R¹⁰, -(Het)-(CH₂)ₘ—C(═O)NR⁷R¹⁰, -(Het)-(CH₂)ₘ-(Z)_{g}-R⁷, -(Het)-(CH₂)ₘ—NR¹⁰—CH₂(CHOR⁸)(CHOR⁸)ₙ—CH₂OR⁸, -(Het)-(CH₂)ₘ—CO₂R⁷, -(Het)-(CH₂)m-NR¹²R¹², -(Het)-(CH₂)ₙ—NR¹²R¹², -(Het)-(CH₂)ₘ-(Z)_{g}R¹², -(Het)-(CH₂)ₘNR¹¹R¹¹, -(Het)-(CH₂)ₘ—N^{⊕}—(R¹¹)₃, -(Het)-(CH₂)ₘ-(Z)_{g}-(CH₂)ₘ—NR¹⁰OR¹⁰, -(Het)-(CH₂CH₂O)ₘ—CH₂CH₂NR¹²R¹², -(Het)-(CH₂)ₘ—(C═O)NR¹²R¹², -(Het)-(CH₂)ₘ—(CHOR⁸)ₘCH₂NR¹⁰-(Z)_{g}-R¹⁰, -(Het)-(CH₂)ₘ—NR¹⁰—(CH₂)ₘ—(CHOR⁸)ₙCH₂NR¹⁰-(Z)_{g}-R¹⁰,
wherein when two —CH₂OR⁸ groups are located 1,2- or 1,3- with respect to each other the R⁸ groups may be joined to form a cyclic mono- or di-substituted 1,3-dioxane or 1,3-dioxolane,
—(CH₂)ₙ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—O—(CH₂)ₘ(CHOR⁸)(CHOR⁸)ₙ—CH₂0R⁸, with the proviso that at least two —CH₂OR⁸ are located adjacent to each other and the R⁸ groups are joined to form a cyclic mono- or di-substituted 1,3-dioxane or 1,3-dioxolane,
—(CH₂)ₙ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane, or
—O—(CH₂)ₘ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane;
wherein each R^{5′} is also, independently,
Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ-(Z)_{g}-CAP, Link —(CH₂)ₙ(Z)_{g}-(CH₂)ₘ—CAP, Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}-CAP, Link —(CH₂)ₙNR¹³—(CH₂)ₘ(CHOR⁸)ₙCH₂NR¹³-(Z)_{g}-CAP, Link —(CH₂)ₘ-(Z)_{g}-(CH₂)ₘ—CAP, Link NH—C(═O)—NH—(CH₂)ₘ—CAP, Link —(CH₂)ₘ—C(═O)NR¹³—(CH₂)ₘ—C(═O)NR¹⁰OR¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)m-Het-(CH₂)ₘ—CAP;
wherein Link is, independently,
—O—, (CH₂)ₙ—, —O(CH₂)ₘ, —NR¹³—C(═O)—NR¹³, —NR¹³—C(═O)—(CH₂)ₘ—, —C(═O)NR¹³—(CH₂)ₘ, —(CH₂)ₙ-Z_{g}-(CH₂)ₙ, —S—, —SO—, —SO₂—, SO₂NR⁷—, SO₂NR¹⁰—, -Het-;
wherein each CAP is, independently, thiazolidinedione, oxazolidinedione, heteroaryl-C(═O)NR²³R¹³, heteroaryl-CAP, —CN, —O—C(═S)NR¹³R¹³, -Z_{g}R¹³, —CR¹⁰(Z_{g}R¹³)(Z_{g}R¹³), —C(═O)NR¹³Ar, imidazoline, tetrazole, tetrazole amide, —SO₂NHR¹³, —SO₂NH—C(R¹³R¹³)-(Z)_{g}-R¹³, cyclic sugars and oligosaccharides, including cyclic amino sugars and oligosaccharides, 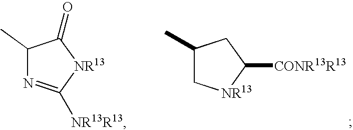
wherein Ar is, independently, phenyl; Substituted phenyl, wherein said substituent is 1-3 groups selected, independently, from OH, OCH₃, NR¹³R¹³, Cl, F, CH₃; heteroaryl, e.g., pyridine, pyrazine, tinazine, furyl, furfuryl-, thienyl, tetrazole, thiazolidinedione and imidazoyl ( 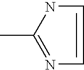
   ) and other heteroaromatic ring systems as defined below;
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
wherein when two —(CH₂OR⁸ groups are located 1,2- or 1,3- with respect to each other the R⁸ groups may be joined to form a cyclic mono- or di-substituted 1,3-dioxane or 1,3-dioxolane;
each R^{6′} is, independently, —R^{5′}, —R⁷, —OR⁸, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 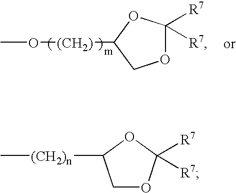
wherein when two —CH₂OR⁸ groups are located 1,2- or 1,3- with respect to each other the R⁸ groups may be joined to form a cyclic mono- or di-substituted 1,3-dioxane or 1,3-dioxolane;
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 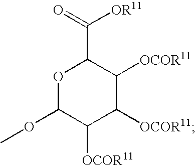
each R⁹ is, independently, —CO₂R¹³, —CON(R¹³)₂, —SO₂CH₂R¹³, or
each R¹⁰ is, independently, —H, —SO₂CH₃, —CO₂R¹³, —C(═O)NR¹³R¹³, C(═O)R¹³, or —(CH₂)ₘ—(CHOH)ₙ—CH₂OH;
each Z is, independently, CHOH, C(═O), —(CH₂)ₙ—, CHNR¹³R¹³, C═NR¹³, or NR¹³;
each R¹¹ is, independently, lower alkyl;
each R¹² is independently, —SO₂CH₃, —CO₂R¹³, —C(═O)NR¹³R¹³, —C(═O)R¹³, or —CH₂—(CHOH)ₙ—CH₂OH;
each R¹³ is, independently, hydrogen, R⁷, R¹⁰, —(CH₂)ₘ—NR¹³R¹³, 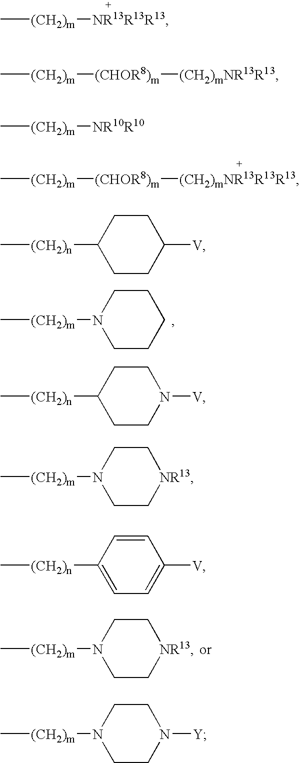
with the proviso that NR¹³R¹³ can be joined on itself to form a ring comprising one of the following: 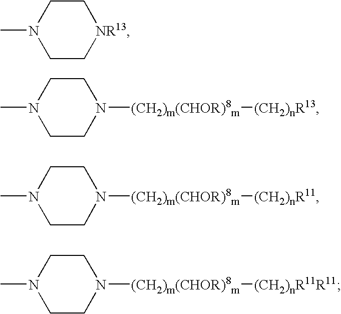
each Het is independently, —NR¹³, —S—, —SO—, or —SO₂—; —O—, —SO₂NR¹³—, —NHSO₂—, —NR¹³CO—, —CONR¹³—;
each g is, independently, an integer from 1 to 6;
each m is, independently, an integer from 1 to 7;
each n is, independently, an integer from 0 to 7;
each Q′ is, independently, —CR^{6′}R^{5′}, —CR^{6′}R^{6′}, N, —NR₃, —S—, —SO—, or —SO₂—;
wherein at most three Q′ in a ring contain a heteroatom and at least one Q′ must be —CR^{5′}R^{6′} or NR^{5′};
each V is, independently, —(CH₂)ₘ—NR⁷R¹⁰, —(CH₂)ₘ—NR⁷R⁷, —(CH₂)ₘ— 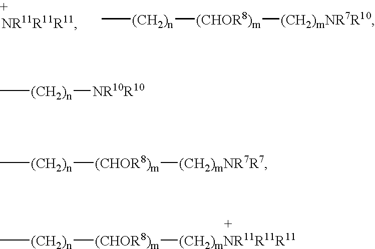
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3- with respect to each other the R⁸ groups may be joined to form a cyclic mono- or di-substituted 1,3-dioxane or 1,3-dioxolane;
or a pharmaceutically acceptable salt thereof, to an individual in need of prophylactic treatment against infection from one or more airborne pathogens.

In another embodiment, a prophylactic treatment method is provided for reducing the risk of infection from an airborne pathogen which can cause a disease in a human, said method comprising administering an effective amount of a sodium channel blocker of Formula I, II or III, or a pharmaceutically acceptable salt thereof, to the lungs of the human who may be at risk of infection from the airborne pathogen but is asymptomatic for the disease, wherein the effective amount of a sodium channel blocker or a pharmaceutically acceptable salt is sufficient to reduce the risk of infection in the human.

In another embodiment, a post-exposure prophylactic treatment or therapeutic treatment method is provided for treating infection from an airborne pathogen comprising administering an effective amount of a sodium channel blocker of Formula I, II or III, or a pharmaceutically acceptable salt thereof to the lungs of an individual in need of such treatment against infection from an airborne pathogen.

### DETAILED DESCRIPTION OF THE PREFERRED EMBODIMENTS

The prophylactic or therapeutic treatment methods of the present invention may be used in situations where a segment of the population has been, or is believed to have been, exposed to one or more airborne pathogens. The prophylactic or therapeutic treatment methods may additionally be used in situations of ongoing risk of exposure to or infection from airborne pathogens. Such situations may arise due to naturally occurring pathogens or may arise due to a bioterrorism event wherein a segment of the population is intentionally exposed to one or more pathogens. The individuals or portion of the population believed to be at risk from infection can be treated according to the methods disclosed herein. Such treatment preferably will commence at the earliest possible time, either prior to exposure if imminent exposure to a pathogen is anticipated or possible or after the actual or suspected exposure. Typically, the prophylactic treatment methods will be used on humans asymptomatic for the disease for which the human is believed to be at risk. The term “asymptomatic” as used herein means not exhibiting medically recognized symptoms of the disease, not yet suffering from infection or disease from exposure to the airborne pathogens, or not yet testing positive for a disease. The treatment methods may involve post-exposure prophylactic or therapeutic treatment, as needed.

Many of the pathogenic agents identified by NIAID have been or are capable of being aerosolized such that they may enter the body through the mouth or nose, moving into the bodily airways and lungs. These areas of the body have mucosal surfaces which naturally serve, in part, to defend against foreign agents entering the body. The mucosal surfaces at the interface between the environment and the body have evolved a number of “innate defense”, i.e., protective mechanisms. A principal form of such innate defense is to cleanse these surfaces with liquid. Typically, the quantity of the liquid layer on a mucosal surface reflects the balance between epithelial liquid secretion, often reflecting anion (Cl⁻ and/or HCO₃⁻) secretion coupled with water (and a cation counter-ion), and epithelial liquid absorption, often reflecting Na⁺ absorption, coupled with water and counter anion (Cl⁻ and/or HCO₃⁻).

R. C. Boucher, in U.S. Pat. No. 6,264,975, describes methods of hydrating mucosal surfaces, particularly nasal airway surfaces, by administration of pyrazinoylguanidine sodium channel blockers. These compounds, typified by amiloride, benzamil and phenamil, are effective for hydration of the mucosal surfaces. U.S. Pat. No. 5,656,256, describes methods of hydrating mucous secretions in the lungs by administration of benzamil or phenamil, for example, to treat diseases such as cystic fibrosis and chronic bronchitis. U.S. Pat. No. 5,725,842 is directed to methods of removing retained mucus secretions from the lungs by administration of amiloride.

It has now been discovered that certain sodium channel blockers which are classes of pyrazinoylguanidine compounds described and exemplified herein as Formulas I, II and III, and in U.S. Provisional Patent Applications 60/495,725, filed Aug. 19, 2003, 60/495,712, filed Aug. 19, 2003 and 60/495,720, filed Aug. 19, 2003, incorporated herein in their entirety, may be used in prophylactic treatment methods to protect humans in whole or in part, against the risk of infection from pathogens which may or may not have been purposely introduced into the environment, typically into the air, of a populated area. Such treatment may be effectively used to protect those who may have been exposed where a vaccine is not available or has not been provided to the population exposed and/or in situations where treatments for the infection resulting from the pathogen to which a population has been subjected are insufficient or unavailable altogether.

Without being bound by any theory, it is believed that the sodium channel blockers disclosed herein surprisingly may be used on substantially normal or healthy lung tissue to prevent or reduce the uptake of airborne pathogens and/or to clear the lungs of all or at least a portion of such pathogens. Preferably, the sodium channel blockers will prevent or reduce the viral or bacterial uptake of airborne pathogens. The ability of sodium channel blockers to hydrate mucosal surfaces is believed to function to first hydrate lung mucous secretions, including mucous containing the airborne pathogens to which the human has been subjected, and then facilitate the removal of the lung mucous secretions from the body. By functioning to remove the lung mucous secretions from the body, the sodium channel blocker thus prevents or, at least, reduces the risk of infection from the pathogen(s) inhaled or brought into the body through a bodily airway.

The present invention is concerned primarily with the prophylactic, post exposure, rescue and therapeutic treatment of human subjects, but may also be employed for the treatment of other mammalian subjects, such as dogs and cats, for veterinary purposes, and to the extent the mammals are at risk of infection or disease from airborne pathogens.

The term “airway” as used herein refers to all airways in the respiratory system such as those accessible from the mouth or nose, including below the larynx and in the lungs, as well as air passages in the head, including the sinuses, in the region above the larynx.

The terms “pathogen” and “pathogenic agent” are interchangeable and, as used herein, means any agent that can cause disease or a toxic substance produced by a pathogen that causes disease. Typically, the pathogenic agent will be a living organism that can cause disease. By way of example, a pathogen may be any microorganism such as bacterium, protozoan or virus that can cause disease.

The term “airborne pathogen” means any pathogen which is capable of being transmitted through the air and includes pathogens which travel through air by way of a carrier material and pathogens either artificially aerosolized or naturally occurring in the air.

The term “prophylactic” as used herein means the prevention of infection, the delay of infection, the inhibition of infection and/or the reduction of the risk of infection from pathogens and includes pre- and post-exposure to pathogens. The prophylactic effect may, inter alia, involve a reduction in the ability of pathogens to enter the body, or may involve the removal of all or a portion of pathogens which reach airways and airway surfaces in the body from the body prior to the pathogens initiating or causing infection or disease. The airways from which pathogens may be removed, in whole or part, include all bodily airways and airway surfaces with mucosal surfaces, including airway surfaces in the lungs.

The term “therapeutic” as used herein means to alleviate disease or infection from pathogens.

The compounds useful in this invention include sodium channel blockers such as those represented by Formulas I, II and III. The sodium channel blockers disclosed may be prepared by the procedures described herein, in combination with procedures known to those skilled in the art.

The term sodium channel blocker as used herein includes the free base and pharmaceutically acceptable salts thereof. Pharmaceutically acceptable salts are salts that retain the desired biological activity of the parent compound and do not impart undesired toxicological effects. Examples of such salts are (a) acid addition salts formed with inorganic acids, for example, hydrochloric acid, hydrobromic acid, sulfuric acid, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid and the like; and (c) salts formed from elemental anions for example, chlorine, bromine, and iodine.

It is to be noted that all enantiomers, diastereomers, and racemic mixtures of compounds within the scope of formulas (I), (II) and (III) are embraced by the present invention and are included within any reference to Formulas (I), (II) or (III) or compounds thereof. Additionally, all mixtures of such enantiomers and diastereomers are within the scope of the present invention and are included within any reference to Formulas (I), (II) or (III) or compounds thereof.

In the compounds represented by these formulas, examples of halogen include fluorine, chlorine, bromine, and iodine. Chlorine and bromine are the preferred halogens. Chlorine is particularly preferred. This description is applicable to the term “halogen” as used throughout the present disclosure.

As used herein, the term “lower alkyl” means an alkyl group having less than 8 carbon atoms. This range includes all specific values of carbon atoms and subranges there between, such as 1, 2, 3, 4, 5, 6, and 7 carbon atoms. The term “alkyl” embraces all types of such groups, e.g., linear, branched, and cyclic alkyl groups. This description is applicable to the term “lower alkyl” as used throughout the present disclosure. Examples of suitable lower alkyl groups include methyl, ethyl, propyl, cyclopropyl, butyl, isobutyl, etc.

As to Formula I, Y may be hydrogen, hydroxyl, mercapto, lower alkoxy, lower alkyl-thio, halogen, lower alkyl, lower cycloalkyl, mononuclear aryl, or —N(R²)₂. The alkyl moiety of the lower alkoxy groups is the same as described above. Examples of mononuclear aryl include phenyl groups. The phenyl group may be unsubstituted or substituted as described above. The preferred identity of Y is —N(R²)₂Particularly preferred are such compounds where each R₂ is hydrogen.

R¹ may be hydrogen or lower alkyl. Hydrogen is preferred for R¹.

Each R² may be, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or

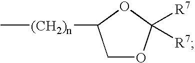

Hydrogen and lower alkyl, particularly C₁-C₃ alkyl are preferred for R² Hydrogen is particularly preferred.

R³ and R⁴ may be, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, provided that at least one of R³ and R⁴ is a group represented by formula (A).

Preferred compounds are those where one of R³ and R⁴ is hydrogen and the other is represented by formula (A).

In formula (A), the moiety —(C(R^{L})₂)ₒ-x-(C(R^{L})₂)ₚ— defines an alkylene group bonded to the aromatic ring. The variables o and p may each be an integer from 0 to 10, subject to the proviso that the sum of o and p in the chain is from 1 to 10. Thus, o and p may each be 0, 1, 2, 3, 4, 5, 6, 7, 8, 9, or 10. Preferably, the sum of o and p is from 2 to 6. In a particularly preferred embodiment of Formula I, the sum of o and p is 4.

The linking group in the alkylene chain, x, may be, independently, O, NR¹⁰, C(═O), CHOH, C(═N—R¹⁰), CHNR⁷R¹⁰, or represents a single bond; therefore, when x represents a single bond, the alkylene chain bonded to the ring is represented by the formula —(C(R^{L})₂)ₒ₊ₚ—, in which the sum o+p is from 1 to 10.

Each R^{L} in Formula I may be, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)m(CHOR⁸)(CHOR⁸)ₙCH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₘ—CO₂R⁷, —O—(CH₂)ₘ—CO₂R⁷, —OSO₃H, —O-glucuronide, —O-glucose,

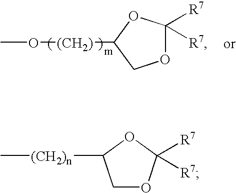

The preferred R^{L} groups for Formula I include —H, —OH, —N(R⁷)₂, especially where each R⁷ is hydrogen.

In the alkylene chain in formula (A), it is preferred that when one R^{L} group bonded to a carbon atoms is other than hydrogen, then the other R^{L} bonded to that carbon atom is hydrogen, i.e., the formula —CHR^{L}—. It is also preferred that at most two R^{L} groups in an alkylene chain are other than hydrogen, where in the other R^{L} groups in the chain are hydrogens. Even more preferably, only one R^{L} group in an alkylene chain is other than hydrogen, where in the other R^{L} groups in the chain are hydrogens. In these embodiments, it is preferable that x represents a single bond.

In another particular embodiment of Formula I, all of the R^{L} groups in the alkylene chain are hydrogen. In these embodiments, the alkylene chain is represented by the formula
—(CH₂)ₒ—x—(CH₂)ₚ—.

In Formula I, each R⁵ is, independently, Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ-(Z)_{g}-CAP, Link —(CH₂)ₙ(Z)_{g}-(CH₂)ₘ—CAP, Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}-CAP, Link —(CH₂)ₙNR¹³—(CH₂)ₘ(CHOR⁸)ₙCH₂NR¹³-(Z)_{g}-CAP, Link —(CH₂)ₘ-(Z)_{g}-(CH₂)ₘ—CAP, Link NH—C(═O)—NH—(CH₂)ₘ—CAP, Link —(CH₂)ₘ—C(═O)NR¹³—(CH₂)ₘ—C(═O)NR¹⁰R¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)ₘ-Het-(CH₂)ₘ—CAP;
wherein Link is, independently, —O—, (CH₂)ₙ—, —O(CH₂)ₘ—, —NR¹³—C(═O)—NR¹³, —NR¹³—C(═O)—(CH₂)ₘ—, —C(═O)NR¹³—(CH₂)ₘ, —(CH₂)ₙ-Z_{g}-(CH₂)ₙ, —S—, —SO—, —SO₂—, SO₂NR⁷—, SO₂NR¹⁰—, -Het-;
wherein each CAP is, independently, thiazolidinedione, oxazolidinedione, heteroaryl-C(═O)NR¹³R¹³, heteroaryl-CAP, —CN, —O—C(═S)NR¹³R¹³, -Z_{g}R¹³, —CR¹⁰(Z_{g}R¹³)(Z_{g}R¹³), —C(═O)OAr, —C(═O)NR¹³Ar, imidazoline, tetrazole, tetrazole amide, —SO₂NHR¹³, —SO₂NH—C(R¹³R¹³)-(Z)_{g}-R¹³, cyclic sugars and oligosaccharides, including cyclic amino sugars and oligosaccharides, 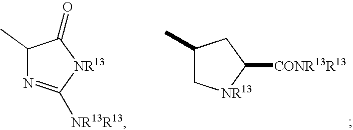
wherein Ar is, independently, phenyl; Substituted phenyl, wherein said substituent is 1-3 groups selected, independently, from OH, OCH₃, NR¹³R¹³, Cl, F, CH₃; heteroaryl, e.g., pyridine, pyrazine, tinazine, furyl, furfuryl-, thienyl, tetrazole, thiazolidinedione and imidazoyl ( 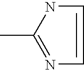
   ) and other heteroaromatic ring systems as defined below;
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
each R⁶ is, independently, —R⁷, —OR⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O -glucose, 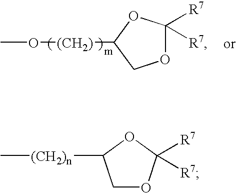
where when two R⁶ are —OR¹¹ and are located adjacent to each other on a phenyl ring, the alkyl moieties of the two R⁶ may be bonded together to form a methylenedioxy group;
with the proviso that when at least two —CH₂OR⁸ are located adjacent to each other, the R⁸ groups may be joined to form a cyclic mono- or di-substituted 1,3-dioxane or 1,3-dioxolane.

In addition, one of more of the R⁶ groups can be one of the R⁵ groups which fall within the broad definition of R⁶ set forth above.

When two R⁶ are —OR¹¹ and are located adjacent to each other on a phenyl ring, the alkyl moieties of the two R⁶ groups may be bonded together to form a methylenedioxy group, i.e., a group of the formula —O—CH₂—O—.

As discussed above, R⁶ may be hydrogen. Therefore, 1, 2, 3, or 4 R⁶ groups may be other than hydrogen. Preferably at most 3 of the R⁶ groups are other than hydrogen.

Each g is, independently, an integer from 1 to 6. Therefore, each g may be 1, 2, 3, 4, 5, or 6.

Each m is an integer from 1 to 7. Therefore, each m may be 1, 2, 3, 4, 5, 6, or 7.

Each n is an integer from 0 to 7. Therefore, each n may be 0, 1, 2, 3, 4, 5, 6, or 7.

Each Q in formula (A) is C—R⁵, C—R⁶, or a nitrogen atom, where at most three Q in a ring are nitrogen atoms. Thus, there may be 1, 2, or 3 nitrogen atoms in a ring. Preferably, at most two Q are nitrogen atoms. More preferably, at most one Q is a nitrogen atom. In one particular embodiment, the nitrogen atom is at the 3-position of the ring. In another embodiment of the invention, each Q is either C—R⁵ or C—R⁶, i.e., there are no nitrogen atoms in the ring.

More specific examples of suitable groups represented by formula (A) are shown in formulas (B)-(E) below: 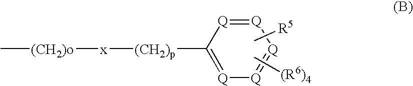
where o, x, p, R⁵, and R⁶, are as defined above; 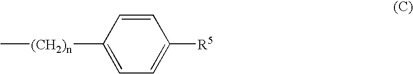
where n is an integer from 1 to 10 and R⁵ is as defined above; 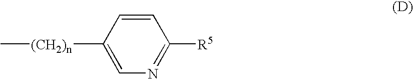
where n is an integer from 1 from 10 and R⁵ is as defined above; 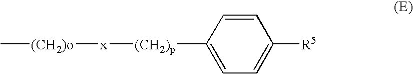
where o, x, p, and R⁵ are as defined above.

In a preferred embodiment of Formula I, Y is —NH₂.

In another preferred embodiment of Formula I, R² is hydrogen.

In another preferred embodiment of Formula I, R¹ is hydrogen.

In another preferred embodiment of Formula I, X is chlorine.

In another preferred embodiment of Formula I, R³ is hydrogen.

In another preferred embodiment of Formula I, R^{L} is hydrogen.

In another preferred embodiment of Formula I, o is 4.

In another preferred embodiment of Formula I, p is 0.

In another preferred embodiment of Formula I, the sum of o and p is 4.

In another preferred embodiment of Formula I, x represents a single bond.

In another preferred embodiment of Formula I, R⁶ is hydrogen.

In another preferred embodiment of Formula I, at most one Q is a nitrogen atom.

In another preferred embodiment of Formula I, no Q is a nitrogen atom.

In a preferred embodiment of Formula I:
X is halogen;
Y is —NR⁷)₂;
R¹ is hydrogen or C₁-C₃ alkyl;
R² is —R⁷, —R⁷, CH₂OR⁷, or —CO₂R⁷;
R³ is a group represented by formula (A); and R⁴ is hydrogen, a group represented by formula (A), or lower alkyl.

In another preferred embodiment of Formula I:
X is chloro or bromo;
Y is —N(R⁷)₂;
R² is hydrogen or C₁-C₃ alkyl;
at most three R⁶ are other than hydrogen as described above;
at most three R^{L} are other than hydrogen as described above; and at most 2 Q are nitrogen atoms.

In another preferred embodiment of Formula I:
Y is —NH₂.

In another preferred embodiment of Formula I:
R⁴ is hydrogen;
at most one R^{L} is other than hydrogen as described above;
at most two R⁶ are other than hydrogen as described above; and at most 1 Q is a nitrogen atom.

In another preferred embodiment, the compound of formula (I) is represented by the formula:

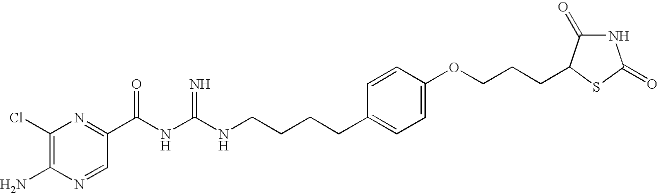

In another preferred embodiment, the compound of formula (I) is represented by the formula:

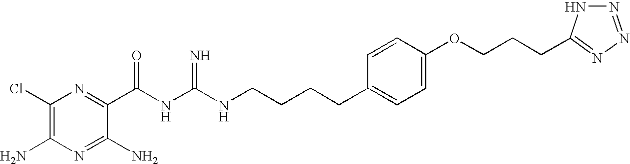

In another preferred embodiment, the compound of formula (I) is represented by the formula:

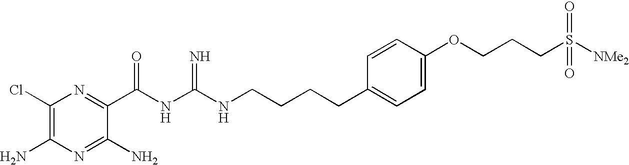

In another preferred embodiment, the compound of formula (I) is represented by the formula:

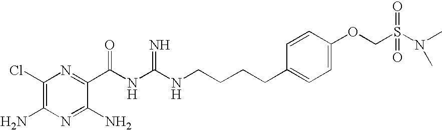

In another preferred embodiment, the compound of formula (I) is represented by the formula:

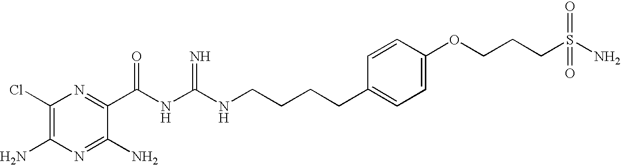

In another preferred embodiment, the compound of formula (I) is represented by the formula:

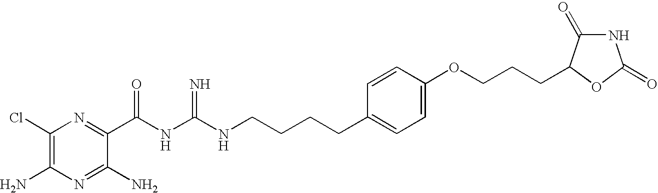

In another preferred embodiment, the compound of formula (I) is represented by the formula:

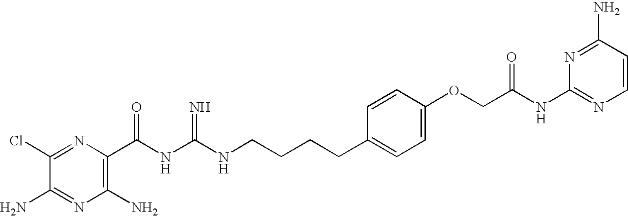

In another preferred embodiment, the compound of formula (I) is represented by the formula:

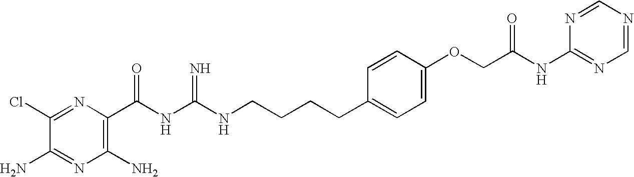

In another preferred embodiment, the compound of formula (I) is represented by the formula:

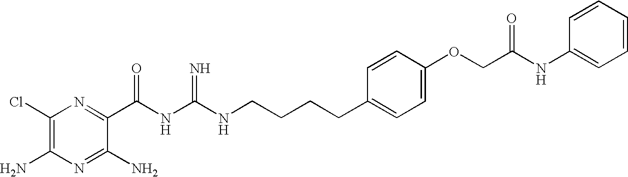

In another preferred embodiment, the compound of formula (I) is represented by the formula:

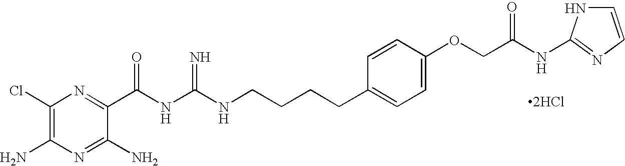

In another preferred embodiment, the compound of formula (I) is represented by the formula:

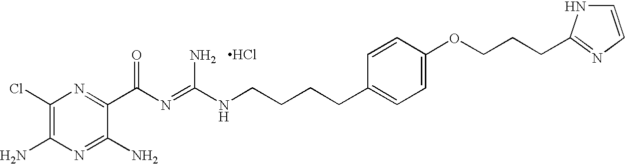

In another preferred embodiment, the compound of formula (I) is represented by the formula:

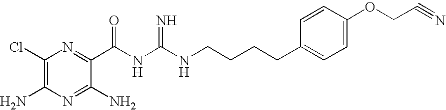

In another preferred embodiment, the compound of formula (I) is represented by the formula:

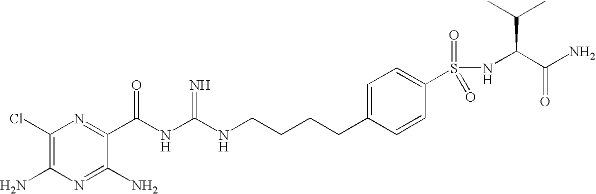

In another preferred embodiment, the compound of formula (I) is represented by the formula:

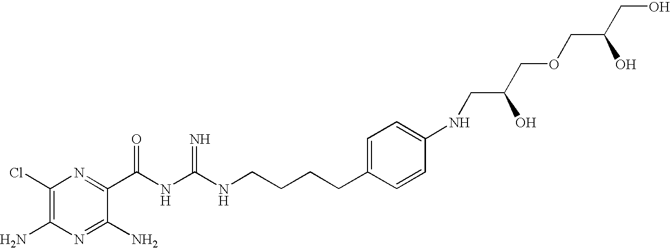

In another preferred embodiment, the compound of formula (I) is represented by the formula:

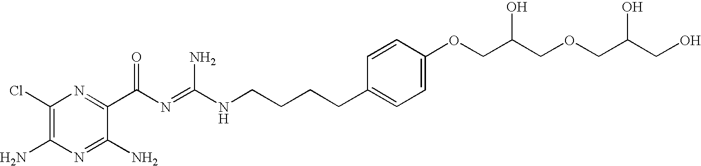

In another preferred embodiment, the compound of formula (I) is represented by the formula:

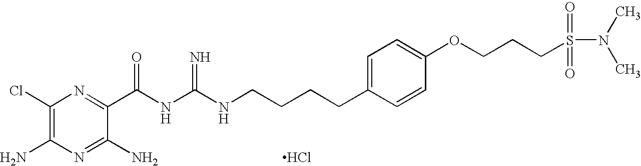

In another preferred embodiment, the compound of formula (I) is represented by the formula:

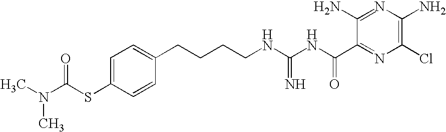

In another preferred embodiment, the compound of formula (I) is represented by the formula:

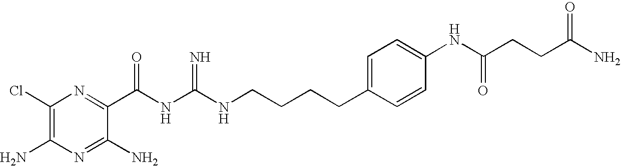

In another preferred embodiment, the compound of formula (I) is represented by the formula:

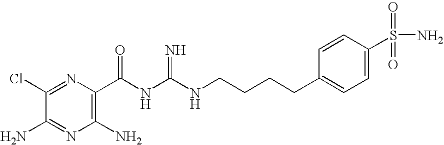

In another preferred embodiment, the compound of formula (I) is represented by the formula:

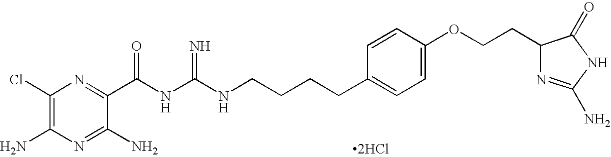

In another preferred embodiment, the compound of formula (I) is represented by the formula:

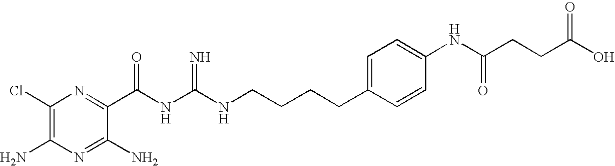

In another preferred embodiment, the compound of formula (I) is represented by the formula:

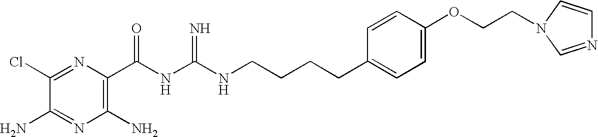

In another preferred embodiment, the compound of formula (I) is represented by the formula:

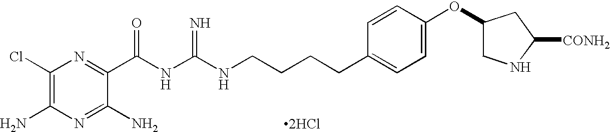

In another preferred embodiment, the compound of formula (I) is represented by the formula:

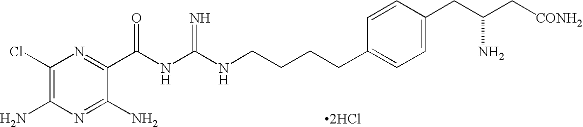

In another preferred embodiment, the compound of formula (I) is represented by the formula:

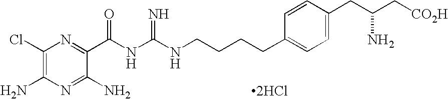

As to Formula II, in a preferred embodiment, each —(CH₂)ₙ-(Z)_{g}-R⁷ falls within the scope of the structures described above and is, independently,
—(CH₂)ₙ—(C═N)—NH₂,
—(CH₂)ₙ—NH—C(═NH)NH₂,
—(CH₂)ₙ—CONHCH₂(CHOH)ₙ—CH₂OH,
—NH—C(═O)—CH₂—(CHOH)ₙ—CH₂OH.

In another a preferred embodiment of Formula II, each —O—(CH₂)ₘ-(Z)_{g}-R⁷ falls within the scope of the structures described above and is, independently,
—O—(CH₂)_{M}—NH—C(═NH)—N(R⁷)₂,
—O—(CH₂)ₘCHNH₂—CO₂NR⁷R¹⁰

In another preferred embodiment of Formula II, each R^{5′} falls within the scope of the structures described above and is, independently,
—O—CH₂CHOHCH₂, O-glucuronide,
—OCH₂CHOHCH₃,
—OCH₂CH₂NH₂,
—OCH₂CH₂NHCO(CH₃)₃,
—CH₂CH₂OH,
—OCH₂CH₂OH,
—O—(CH₂)ₘ-Boc,
—(CH₂)ₘ—Boc,
—OCH₂CH₂OH,
—OCH₂CO₂H,
—O—(CH₂)ₘ—NH—C(═NH)—N(R⁷)₂,
—(CH₂)ₙ—NH—C(═NH)—N(R⁷)₂,
—NHCH₂(CHOH)₂—CH₂OH,
—OCH₂CO₂Et,
—NHSO₂CH₃,
—(CH₂)ₘNH—C(═O)—OR⁷,
—O—(CH₂)ₘ—NH—C(═O)—OR⁷,
—(CH₂)ₙ—NH—C(═O)—R¹¹,
—O—(CH₂)ₘ—NH—C(═O)—R¹¹,
—O—CH₂C(═O)NH₂,
—CH₂NH₂,
—NHCO₂Et,
—OCH₂CH₂CH₂CH₂OH,
—CH₂NHSO₂CH₃,
—OCH₂CH₂CHOHCH₂OH,
—OCH₂CH₂NHCO₂Et,
—NH—C(═NH₂)—NH₂,
—OCH₂-(α-CHOH)₂—CH₂OH
—OCH₂CHOHCH₂NH₂, 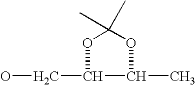
—(CH₂)ₘ—CHOH—CH₂—NHBOC,
—O—(CH₂)ₘ—CHOH—CH₂—NHBoc,
—(CH₂)ₘ—NHC(O)OR⁷,
—O—(CH₂)ₘ—NHC(O)OR⁷,
—OCH₂CH₂CH₂NH₂,
—OCH₂CH₂NHCH₂(CHOH)₂CH₂OH,
—OCH₂CH₂NH(CH₂[(CHOH)₂CH₂OH)]₂,
—(CH₂)₄—NHBoc,
—(CH₂)₄—NH₂,
—(CH₂)₄—OH,
—OCH₂CH₂NHSO₂CH₃,
—O—(CH₂)ₘ—C(═NH)—N(R⁷)₂,
—(CH₂)ₙ—C(═NH)—N(R⁷)₂,
—(CH₂)₃—NH Boc,
—(CH₂)₃NH₂,
—O—(CH₂)ₘ—NH—NH—C(═NH)—N(R⁷)₂,
—(CH₂)ₙ—NH—NH—C(═NH)—N(R₇)₂, or
—O—CH₂—CHOH—CH₂—NH—C(═NH)—N(R⁷)₂;

Preferred examples of R^{5′} in the embodiments of Formula II described above include:
—N(SO₂CH₃)₂,
—CH₂—CHNHBocCO₂CH₃ (α),
—O—CH₂—CHNH₂CO₂H (α),
—O—CH₂—CHNH₂CO₂CH₃ (α),
—O—(CH₂)₂—N⁺(CH₃)₃,
—C(═O)NH—(CH₂)₂—NH₂, and
—C(═O)NH—(CH₂)₂—NH—C(═NH)—NH₂.

Preferred examples of R^{5′} also include:
—N(SO₂CH₃)₂,
—CH₂—CHNHBocCO₂CH₃ (α),
—O—CH₂—CHNH₂CO₂H (α),
—O—CH₂—CHNH₂CO₂CH₃ (α),
—O—(CH₂)₂—N⁺(CH₃)₃,
—C(═O)NH—(CH₂)₂—NH₂,
—C(═O)NH—(CH₂)₂—NH—C(═NH)—NH₂, and

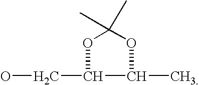

In Formula II, the preferred identity of Y is —N(R²)₂. Particularly preferred are such compounds where each R² is hydrogen.

R¹ in Formula II may be hydrogen or lower alkyl. Hydrogen is preferred for R¹.

R³′ and R⁴′ may be, independently, hydrogen, a group represented by formula (A′), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, provided that at least one of R^{3′} and R^{4′} is a group represented by formula (A′).

Preferred compounds of Formula II are those where one of R^{3′} and R⁴⁰ is hydrogen and the other is represented by formula (A′).

In formula (A′), the moiety —(C(R^{L})₂)ₒ-x-(C(R^{L})₂)ₚ— defines an alkylene group. The variables o and p may each be an integer from 0 to 10, subject to the proviso that the sum of o and p in the chain is from 1 to 10. Thus, o and p may each be 0, 1, 2, 3, 4, 5, 6, 7, 8, 9, or 10. Preferably, the sum of o and p is from 2 to 6. In a particularly preferred embodiment, the sum of o and p is 4.

The linking group in the alkylene chain of Formula II, x, may be, independently, O, NR¹⁰, C(═O), CHOH, C(═N—R¹⁰), CHNR⁷R¹⁰, or represents a single bond; therefore, when x represents a single bond, the alkylene chain bonded to the ring is represented by the formula —(C(R^{L})₂)ₒ₊ₚ—, in which the sum o+p is from 1 to 10.

The preferred R^{L} groups in Formula II include —H, —OH, —N(R⁷)₂, especially where each R⁷ is hydrogen.

In the alkylene chain in formula (A′), it is preferred that when one R^{L} group bonded to a carbon atoms is other than hydrogen, then the other R^{L} bonded to that carbon atom is hydrogen, i.e., the formula —CHR^{L}—. It is also preferred that at most two R^{L} groups in an alkylene chain are other than hydrogen, where in the other R^{L} groups in the chain are hydrogens. Even more preferably, only one R^{L} group in an alkylene chain is other than hydrogen, where in the other R^{L} groups in the chain are hydrogens. In these embodiments, it is preferable that x represents a single bond.

In another particular embodiment of Formula II, all of the R^{L} groups in the alkylene chain are hydrogen. In these embodiments, the alkylene chain is represented by the formula
—(CH₂)ₒ-x-(CH₂)ₚ—.

As discussed above, R may be hydrogen. Therefore, 1 or 2 R^{6′} groups may be other than hydrogen. Preferably at most 3 of the R^{6′} groups are other than hydrogen.

Each g is, independently, an integer from 1 to 6. Therefore, each g may be 1, 2,3,4, 5,or 6.

Each m is an integer from 1 to 7. Therefore, each m may be 1, 2, 3, 4, 5, 6, or 7.

Each n is an integer from 0 to 7. Therefore, each n maybe 0, 1, 2, 3, 4, 5, 6, or 7.

In a preferred embodiment of Formula II, Y is —NH₂.

In another preferred embodiment of Formula II, R² is hydrogen.

In another preferred embodiment of Formula II, R¹ is hydrogen.

In another preferred embodiment of Formula II, X is chlorine.

In another preferred embodiment of Formula II, R^{3′} is hydrogen.

In another preferred embodiment of Formula II, R^{L} is hydrogen.

In another preferred embodiment of Formula II, o is 4.

In another preferred embodiment of Formula II, p is 0.

In another preferred embodiment of Formula II, the sum of o and p is 4.

In another preferred embodiment of Formula II, x represents a single bond.

In another preferred embodiment of Formula II, R^{6′} is hydrogen.

In a preferred embodiment of Formula II:
X is halogen;
Y is —N(R⁷)₂;
R¹ is hydrogen or C₁-C₃ alkyl;
R² is —R⁷, —OR⁷, CH₂O⁷, or —CO₂R⁷;
R^{3′} is a group represented by formula (A′); and
R^{4′} is hydrogen, a group represented by formula (A′), or lower alkyl.

In another preferred embodiment of Formula II:
X is chloro or bromo;
Y is —N(R⁷)₂;
R² is hydrogen or C₁-C₃ alkyl;
at most three R^{6′} are other than hydrogen as described above;
at most three R^{L} are other than hydrogen as described above.

In another preferred embodiment of Formula II:
Y is —NH₂.

In another preferred embodiment of Formula II:
R⁴′ is hydrogen;
at most one R^{L} is other than hydrogen as described above;
at most two R⁶ are other than hydrogen as described above.

In another preferred embodiment, formula (II) is represented by the formula:

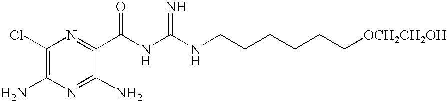

In another preferred embodiment, the compound of formula (II) is represented by the formula:

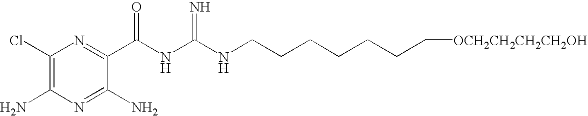

In another preferred embodiment, the compound of formula (II) is represented by the formula:

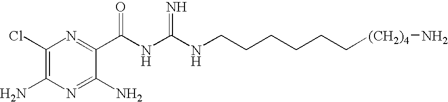

In another preferred embodiment, the compound of formula (II) is represented by the formula:

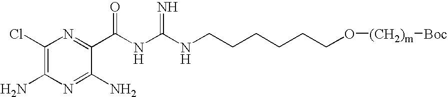

In another preferred embodiment, the compound of formula (II) is represented by the formula:

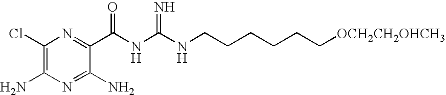

In another preferred embodiment, the compound of formula (II) is represented by the formula:

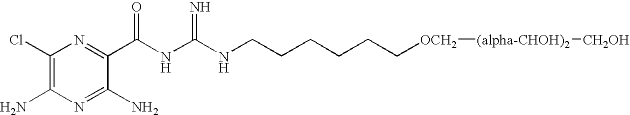

In another preferred embodiment, the compound of formula (II) is represented by the formula:

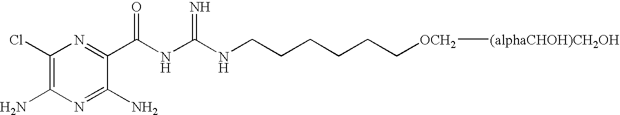

In another preferred embodiment, the compound of formula (II) is represented by the formula:

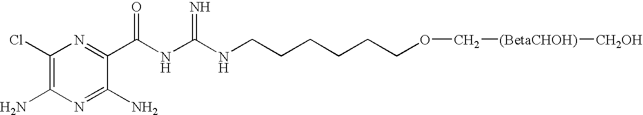

In another preferred embodiment, the compound of formula (II) is represented by the formula:

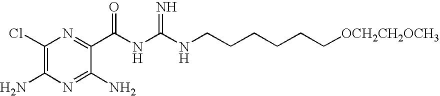

In another preferred embodiment, the compound of formula (II) is represented by the formula:

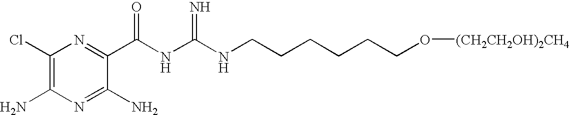

In another preferred embodiment, the compound of formula (II) is represented by the formula:

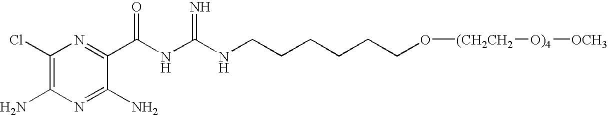

In another preferred embodiment, the compound of formula (II) is represented by the formula:

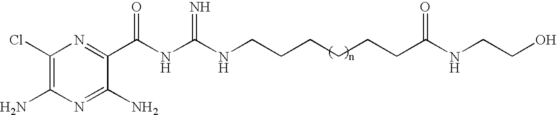

In another preferred embodiment, the compound of formula (II) is represented by the formula:

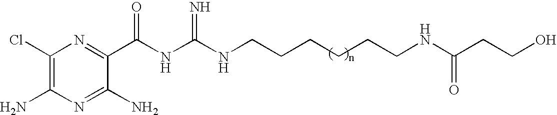

In another preferred embodiment, the compound of formula (II) is represented by the formula:

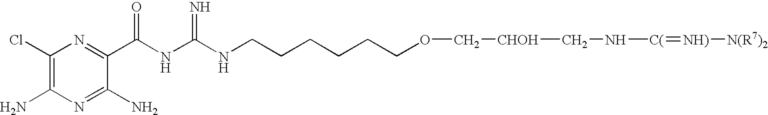

In another preferred embodiment, the compound of formula (II) is represented by the formula:

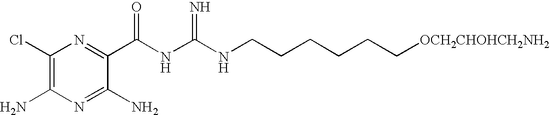

In another preferred embodiment, the compound of formula (II) is represented by the formula:

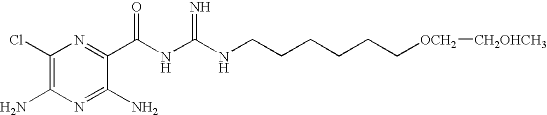

In another preferred embodiment, the compound of formula (II) is represented by the formula:

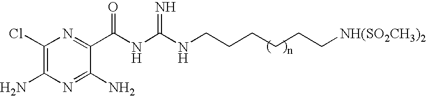

In another preferred embodiment, the compound of formula (II) is represented by the formula:

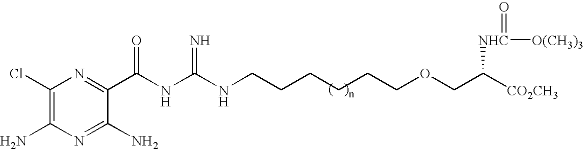

In another preferred embodiment, the compound of formula (II) is represented by the formula:

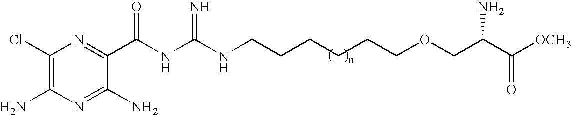

In another preferred embodiment, the compound of formula (II) is represented by the formula:

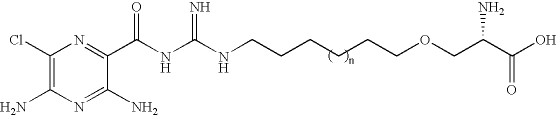

In another preferred embodiment, the compound of formula (II) is represented by the formula:

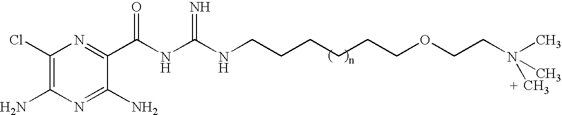

In another preferred embodiment, the compound of formula (II) is represented by the formula:

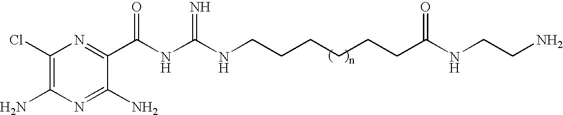

In another preferred embodiment, the compound of formula (II) is represented by the formula:

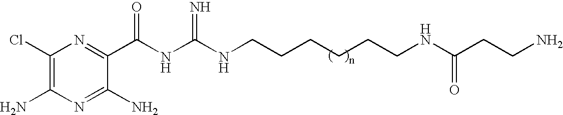

In another preferred embodiment, the compound of formula (II) is represented by the formula:

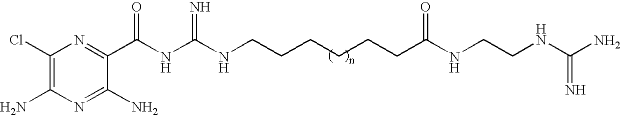

In another preferred embodiment, the compound of formula (II) is represented by the formula:

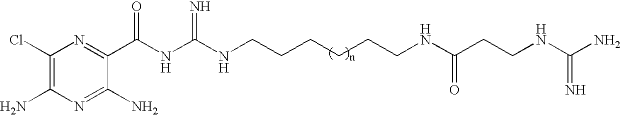

In another preferred embodiment, the compound of formula (II) is represented by the formula:

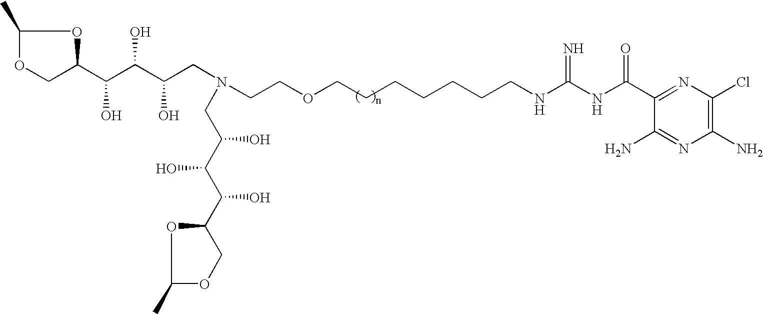

In another preferred embodiment, the compound of formula (II) is represented by the formula:

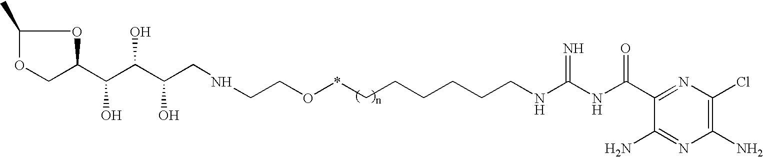

In another preferred embodiment, the compound of formula (II) is represented by the formula:

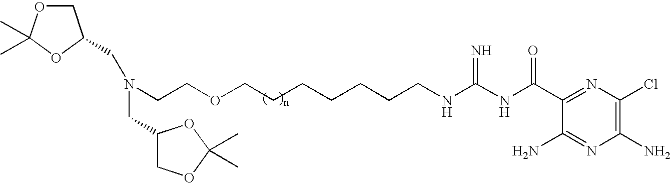

In another preferred embodiment, the compound of formula (II) is represented by the formula:

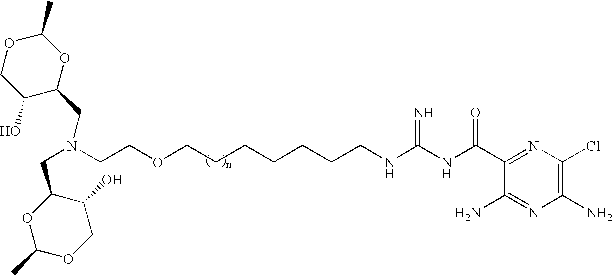

In another preferred embodiment, the compound of formula (II) is represented by the formula:

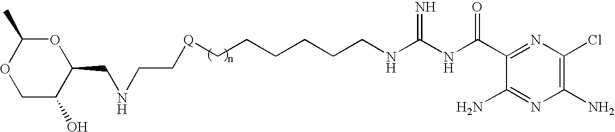

In another preferred embodiment, the compound of formula (II) is represented by the formula:

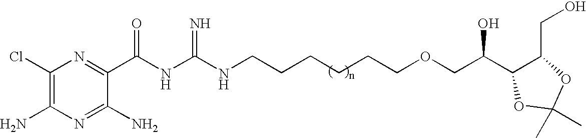

In another preferred embodiment, the compound of formula (II) is represented by the formula:

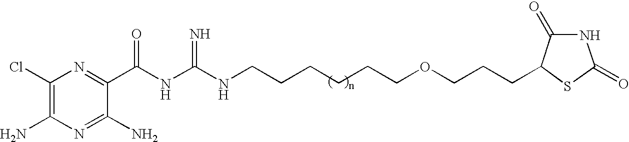

In another preferred embodiment, the compound of formula (II) is represented by the formula:

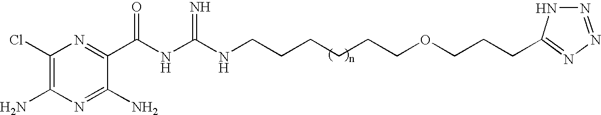

In another preferred embodiment, the compound of formula (II) is represented by the formula:

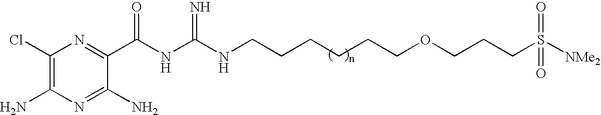

In another preferred embodiment, the compound of formula (II) is represented by the formula:

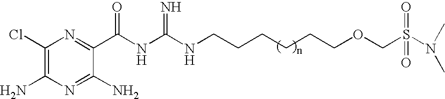

In another preferred embodiment, the compound of formula (II) is represented by the formula:

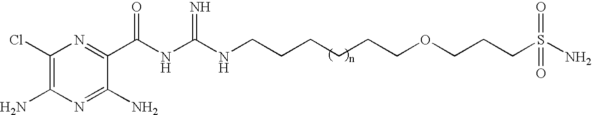

In another preferred embodiment, the compound of formula (II) is represented by the formula:

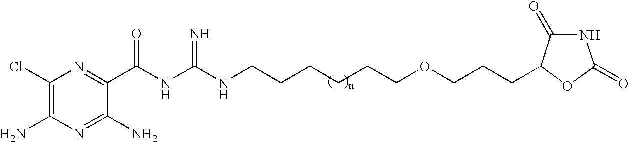

In another preferred embodiment, the compound of formula (II) is represented by the formula:

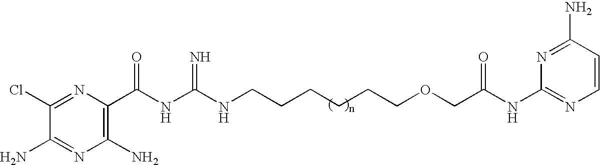

In another preferred embodiment, the compound of formula (II) is represented by the formula:

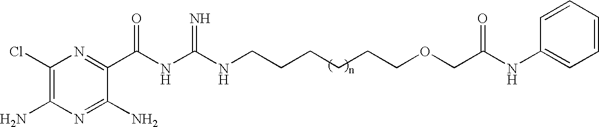

In another preferred embodiment, the compound of formula (II) is represented by the formula:

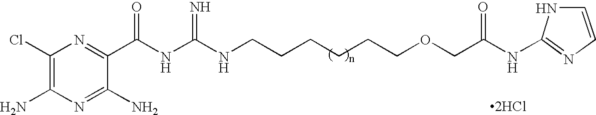

In another preferred embodiment, the compound of formula (II) is represented by the formula:

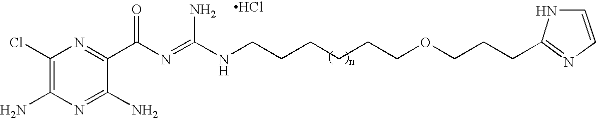

In another preferred embodiment, the compound of formula (II) is represented by the formula:

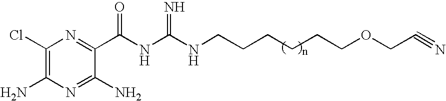

In another preferred embodiment, the compound of formula (II) is represented by the formula:

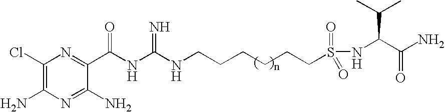

In another preferred embodiment, the compound of formula (II) is represented by the formula:

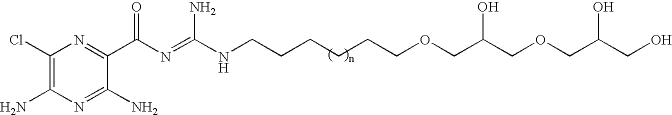

In another preferred embodiment, the compound of formula (II) is represented by the formula:

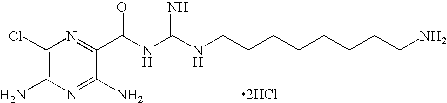

In another preferred embodiment, the compound of formula (II) is represented by the formula:

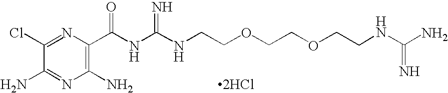

In another preferred embodiment, the compound of formula (II) is represented by the formula:

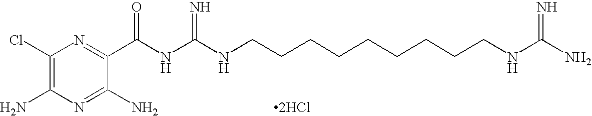

In another preferred embodiment, the compound of formula (II) is represented by the formula:

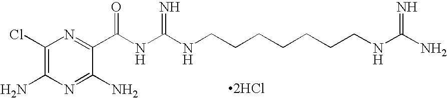

In another preferred embodiment, the compound of formula (II) is represented by the formula:

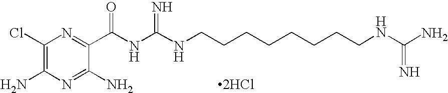

In another preferred embodiment, the compound of formula (II) is represented by the formula:

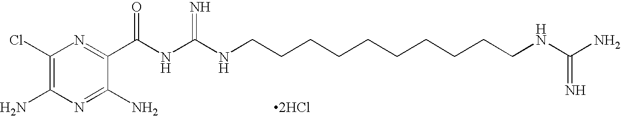

In another preferred embodiment, the compound of formula (II) is represented by the formula:

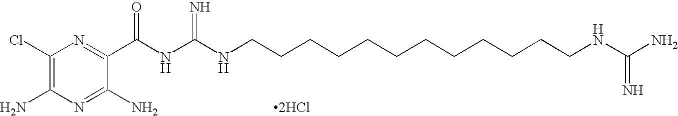

In another preferred embodiment, the compound of formula (II) is represented by the formula:

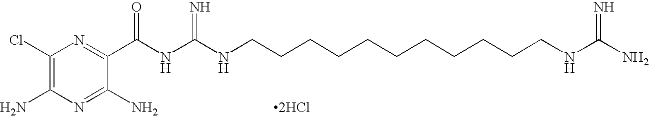

In another preferred embodiment, the compound of formula (II) is represented by the formula:

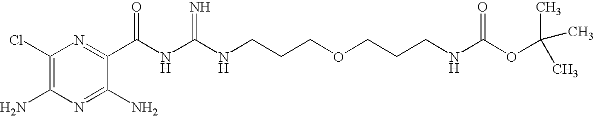

In another preferred embodiment, the compound of formula (II) is represented by the formula:

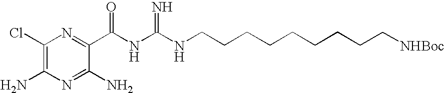

In another preferred embodiment, the compound of formula (II) is represented by the formula:

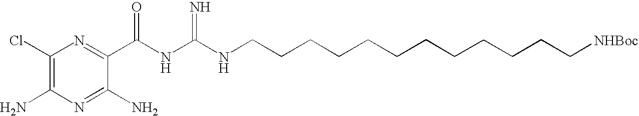

In another preferred embodiment, the compound of formula (II) is represented by the formula:

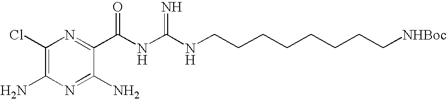

In another preferred embodiment, the compound of formula (II) is represented by the formula:

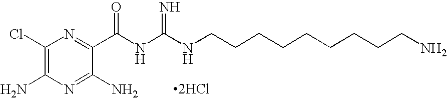

In another preferred embodiment, the compound of formula (II) is represented by the formula:

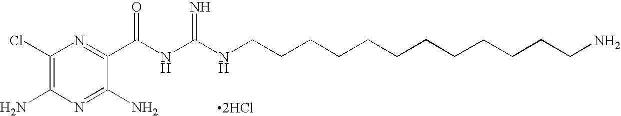

In another preferred embodiment, the compound of formula (II) is represented by the formula:

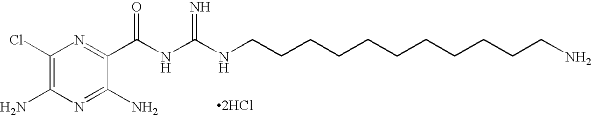

In another preferred embodiment, the compound of formula (II) is represented by the formula:

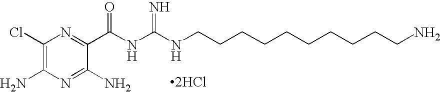

In another preferred embodiment, the compound of formula (II) is represented by the formula:

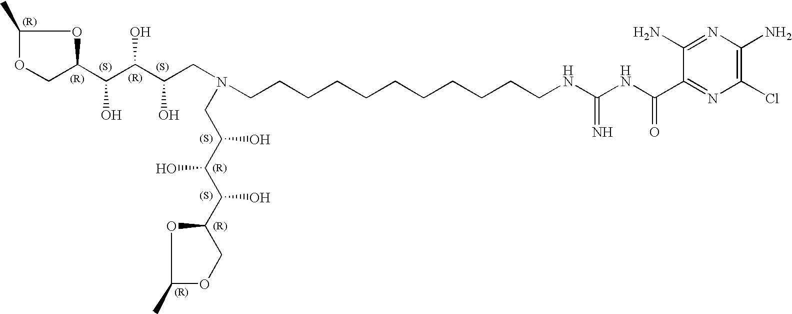

As to Formula III, in a preferred embodiment, each —(CH₂)ₙ-(Z)_{g}-R⁷ falls within the scope of the structures described above and is, independently,
—(CH₂)ₙ—(C═N)—NH₂,
—(CH₂)ₘ—NH—C(═NH)NH₂,
—(CH₂)ₙ—CONHCH₂(CHOH)₂—CH₂OH,
—NH—C(═O)—CH₂—(CHOH)ₙCH₂OH.

In another a preferred embodiment of Formula III, each —O—(CH₂)ₘ-(Z)_{g}R^{7,} falls within the scope of the structures described above and is, independently,
—O—(CH₂)_{M}—NH—C(═NH)—N(R⁷)₂,
—O—(CH₂)ₘ—CHNH₂—CO₂NR⁷R¹⁰

In another preferred embodiment of Formula III, each R^{5′} falls within the scope of the structures described above and is, independently,
—O—CH₂CHOHCH₂O-glucuronide,
—OCH₂CHOHCH₃,
—OCH₂CH₂NH₂,
—OCH₂CH₂NHCO(CH₃)₃,
—CH₂CH₂OH,
—OCH₂CH₂OH,
—O—(CH₂)ₘ-Boc,
—(CH₂)ₘ-Boc,
—OCH₂CH₂OH,
—OCH₂CO₂H,
—O—(CH₂)ₘ—NH—C(═NH)—N(R⁷)₂,
—(CH₂)ₙ—NH—C(═NH)—N(R⁷)₂,
—NHCH₂(CHOH)₂—CH₂OH,
—OCH₂CO₂Et,
—NHSO₂CH₃,
—(CH₂)ₘ—NH—C(═O)—OR⁷,
—O—(CH₂)ₘ—NH—C(═O)—OR⁷,
—(CH₂)ₙ—NH—C(═O)—R¹¹,
—O—(CH₂)ₘ—NH—C(═O)—R¹¹,
—O—CH₂C(═O)NH₂,
—CH₂NH₂,
—NHCO₂Et,
—OCH₂CH₂CH₂CH₂OH,
—CH₂NHSO₂CH₃,
—OCH₂CH₂CHOHCH₂OH,
—OCH₂CH₂NHCO₂Et,
—NH—C(═NH₂)—NH₂,
—OCH₂—(α(—CHOH)₂—CH₂OH
—OCH₂CHOHCH₂NH₂, 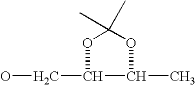
—(CH₂)ₘ—CHOH—CH₂—NHBoc,
—O—(CH₂)ₘ—CHOH—CH₂—NHBoc,
—(CH₂)ₘ—NHC(O)OR⁷,
—O—(CH₂)ₘ—NHC(O)OR⁷,
—OCH₂CH₂CH₂NH₂,
—OCH₂CH₂NHCH₂(CHOH)₂CH₂OH,
—OCH₂CH₂NH(CH₂[(CHOH)₂CH₂OH)]₂,
—(CH₂)₄—NHBoc,
—(CH₂)₄—NH₂,
—(CH₂)₄—OH,
—OCH₂CH₂NHSO₂CH₃,
—O—(CH₂)ₘ—C(═NH)—N(R⁷)₂,
—(CH₂)ₙ—C(═NH)—N(R⁷)₂,
—(CH₂)₃—NH Boc,
—(CH₂)₃NH₂,
—O—(CH₂)ₘ—NH—NH—C(═NH)—N(R⁷)₂,
—(CH₂)ₙ—NH—NH—C(═NH)—N(R⁷)₂, or
—O—CH₂—CHOH—CH₂—NH—C(═NH)—N(R⁷)₂;

Preferred examples of R^{5′} in the embodiments described above include:
—N(SO₂CH₃)₂,
—CH₂—CHNHBocCO₂CH₃ (α),
—O—CH₂—CHNH₂CO₂H (α),
—O—CH₂—CHNH₂CO₂CH₃ (α),
—O—(CH₂)₂—N⁺(CH₃)₃,
—C(═O)NH—(CH₂)₂—NH₂, and
—C(═O)NH—(CH₂)₂—NH—C(═NH)—NH₂.

Preferred examples of R^{5′} also include:
—N(SO₂CH₃)₂,
—CH₂—CHNHBocCO₂CH₃ (α),
—O—CH₂—CHNH₂CO₂H (α),
—O—CH₂—CHNH₂CO₂CH₃ (α),
—O—(CH₂)₂—N⁺(CH₃)₃,
—C(═O)NH—(CH₂)₂—NH₂,
—C(═O)NH—(CH₂)₂—NH—C(═NH)—NH₂, and

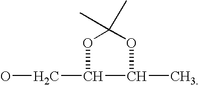

Substituents for the phenyl group where applicable in Formula III include halogens. Particularly preferred halogen substituents are chlorine and bromine.

Y in Formula III may be hydrogen, hydroxyl, mercapto, lower alkoxy, lower alkyl-thio, halogen, lower alkyl, lower cycloalkyl, mononuclear aryl, or —N(R²)₂. The alkyl moiety of the lower alkoxy groups is the same as described above. Examples of mononuclear aryl include phenyl groups. The phenyl group may be unsubstituted or substituted as described above. The preferred identity of Y is —N(R²)₂. Particularly preferred are such compounds where each R² is hydrogen.

R¹ may be hydrogen or lower alkyl in Formula III. Hydrogen is preferred for R¹.

Hydrogen and lower alkyl, particularly C₁-C₃ alkyl are preferred for R² in Formula III. Hydrogen is particularly preferred.

Preferred compounds of Formula III are those where one of R^{3″} and R^{4″} is hydrogen and the other is represented by formula (A″).

In formula (A″), the moiety —(C(R^{L})₂)ₒ-x-(C(R^{L})₂)ₚ— defines an alkylene group bonded to the cyclic ring. The variables o and p may each be an integer from 0 to 10, subject to the proviso that the sum of o and p in the chain is from 1 to 10. Thus, o and p may each be 0, 1, 2, 3, 4, 5, 6, 7, 8, 9, or 10. Preferably, the sum of o and p is from 2 to 6. In a particularly preferred embodiment, the sum of o and p is 4.

The linking group in the alkylene chain, x, may be, independently, O, NR¹⁰, C(═O), CHOH, C(═N—R¹⁰), CHNR⁷R¹⁰, or represents a single bond; therefore, when x represents a single bond, the alkylene chain bonded to the ring is represented by the formula —(C(R^{L})₂)ₒ₊ₚ—, in which the sum o+p is from 1 to 10.

The preferred R^{L} groups in Formula III include —H, —OH, —N(R⁷)₂, especially where each R⁷ is hydrogen.

In the alkylene chain in formula (A″), it is preferred that when one R^{L} group bonded to a carbon atoms is other than hydrogen, then the other R^{L} bonded to that carbon atom is hydrogen, i.e., the formula —CHR^{L}—. It is also preferred that at most two R^{L} groups in an alkylene chain are other than hydrogen, where in the other R^{L} groups in the chain are hydrogens. Even more preferably, only one R^{L} group in an alkylene chain is other than hydrogen, where in the other R^{L} groups in the chain are hydrogens. In these embodiments, it is preferable that x represents a single bond.

In another particular embodiment of the invention, all of the R^{L} groups in the alkylene chain are hydrogen. In these embodiments, the alkylene chain is represented by the formula
—(CH₂)ₒ-x-(CH₂)ₚ—.

Each g is, independently, an integer from 1 to 6. Therefore, each g may be 1, 2, 3, 4, 5, or 6.

Each m is an integer from 1 to 7. Therefore, each m may be 1, 2, 3, 4, 5, 6, or 7.

Each n is an integer from 0 to 7. Therefore, each n maybe 0, 1, 2, 3, 4, 5, 6, or 7.

Each Q′ is, independently, —CHR^{5′}, —CHR^{6′}, —NR⁷, —NR¹⁰, —S—, —SO—, or —SO₂—; wherein at most three Q′ in a ring contain a heteroatom and at least one Q′ must be —CHR^{5′}. Thus, there may be 1, 2, or 3 nitrogen atoms in a ring. Preferably, at most two Q′ are nitrogen atoms.

In a preferred embodiment of Formula III, Y is —NH₂.

In another preferred embodiment Formula III, R² is hydrogen.

In another preferred embodiment Formula III, R¹ is hydrogen.

In another preferred embodiment Formula III, X is chlorine.

In another preferred embodiment Formula III, R^{3′} is hydrogen.

In another preferred embodiment Formula III, R^{L} is hydrogen.

In another preferred embodiment Formula III, o is 4.

In another preferred embodiment Formula III, p is 0.

In another preferred embodiment Formula III, the sum of o and p is 4.

In another preferred embodiment Formula III, x represents a single bond.

In another preferred embodiment Formula III, R^{6′} is hydrogen.

In another preferred embodiment Formula III, at most 2 Q′ are nitrogen atoms.

In another preferred embodiment Formula III, at most one Q′ is a nitrogen atom.

In another preferred embodiment Formula III, no Q′ is a nitrogen atom.

In a preferred embodiment of Formula III:
X is halogen;
Y is —N(R⁷)₂;
R¹ is hydrogen or C₁-C₃ alkyl;
R² is —R⁷, —OR⁷, CH₂O⁷, or —CO₂R⁷;
R^{3″} is a group represented by formula (A″); and
R^{4″} is hydrogen, a group represented by formula (A″), or lower alkyl.

In another preferred embodiment of Formula III:
X is chloro or bromo;
Y is —N(R⁷)₂;
R² is hydrogen or C₁-C₃ alkyl;
at most three R^{6′} are other than hydrogen as described above;
at most three R^{L} are other than hydrogen as described above; and
at most 2 Q′ are nitrogen atoms.

In another preferred embodiment of Formula III:
Y is —NH₂;

In another preferred embodiment of Formula III:
R⁴ is hydrogen;
at most one R^{L} is other than hydrogen as described above;
at most two R^{6′} are other than hydrogen as described above; and
at most 1 Q′ is a nitrogen atom.

In another preferred embodiment of Formula III, the compound is represented by the formula:

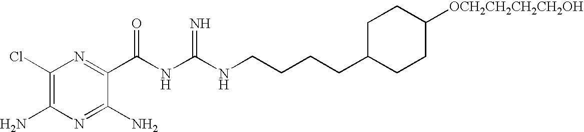

In another preferred embodiment, the compound of formula (III) is represented by the formula:

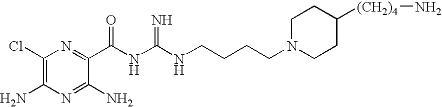

In another preferred embodiment, the compound of formula (III) is represented by the formula:

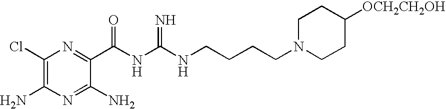

In another preferred embodiment, the compound of formula (III) is represented by the formula:

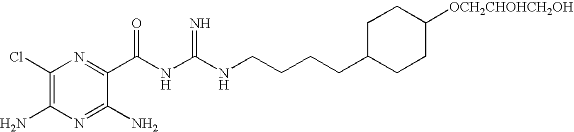

In another preferred embodiment, the compound of formula (III) is represented by the formula:

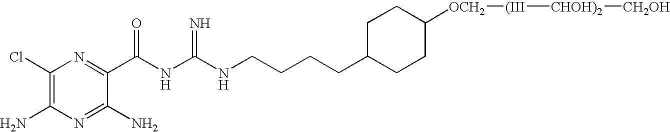

In another preferred embodiment, the compound of formula (III) is represented by the formula:

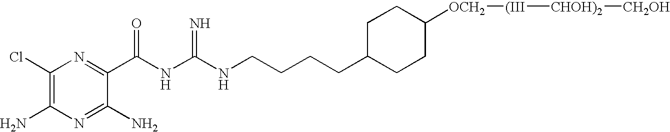

In another preferred embodiment, the compound of formula (III) is represented by the formula:

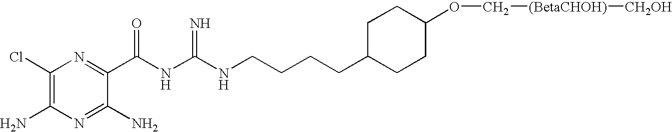

In another preferred embodiment, the compound of formula (III) is represented by the formula:

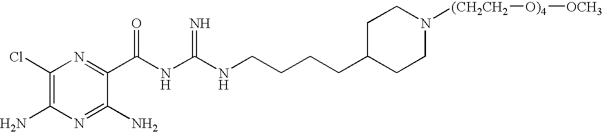

In another preferred embodiment, the compound of formula (III) is represented by the formula:

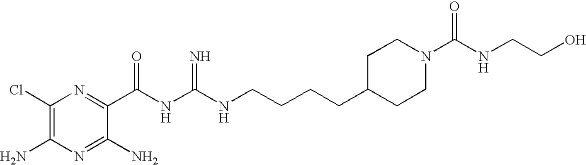

In another preferred embodiment, the compound of formula (III) is represented by the formula:

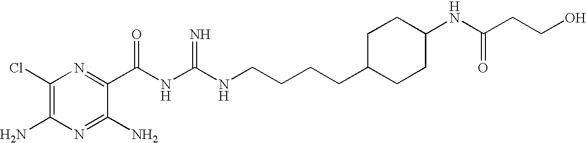

In another preferred embodiment, the compound of formula (III) is represented by the formula:

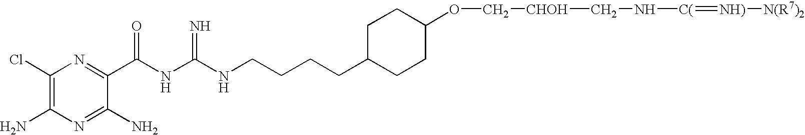

In another preferred embodiment, the compound of formula (III) is represented by the formula:

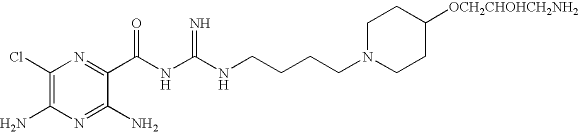

In another preferred embodiment, the compound of formula (III) is represented by the formula:

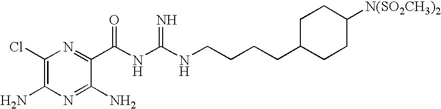

In another preferred embodiment, the compound of formula (III) is represented by the formula:

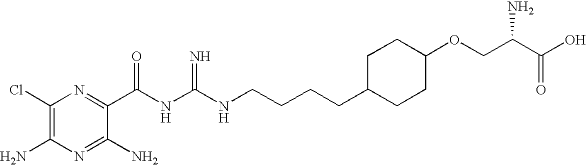

In another preferred embodiment, the compound of formula (III) is represented by the formula:

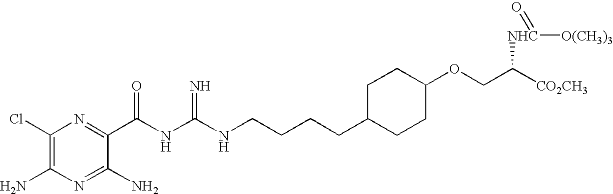

In another preferred embodiment, the compound of formula (III) is represented by the formula:

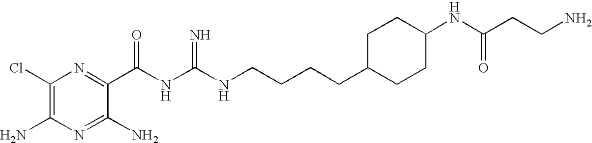

In another preferred embodiment, the compound of formula (III) is represented by the formula:

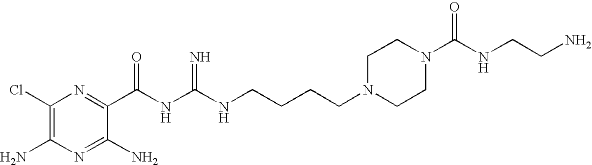

In another preferred embodiment, the compound of formula (III) is represented by the formula:

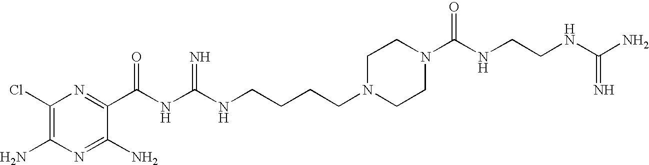

In another preferred embodiment, the compound of formula (III) is represented by the formula:

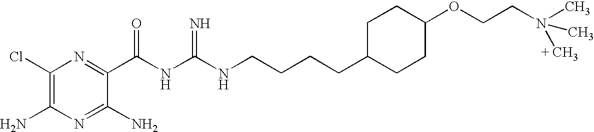

In another preferred embodiment, the compound of formula (III) is represented by the formula:

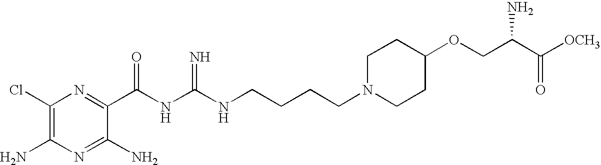

In another preferred embodiment, the compound of formula (III) is represented by the formula:

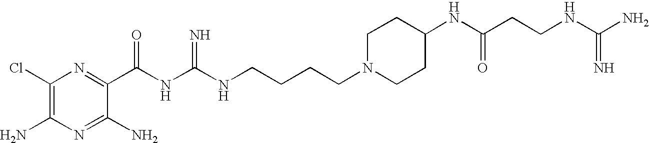

In another preferred embodiment, the compound of formula (III) is represented by the formula:

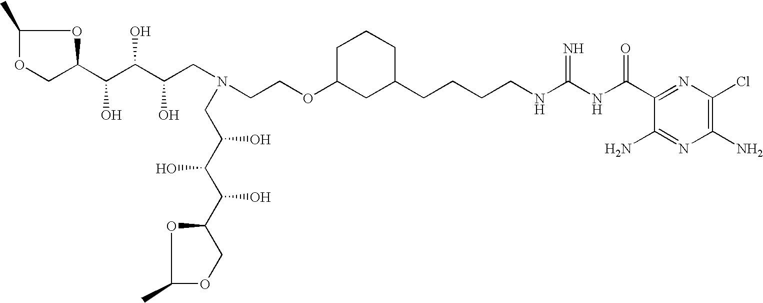

In another preferred embodiment, the compound of formula (III) is represented by the formula:

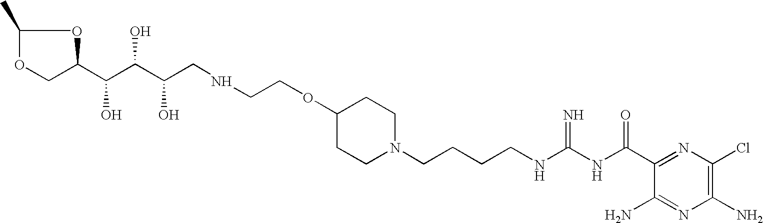

In another preferred embodiment, the compound of formula (III) is represented by the formula:

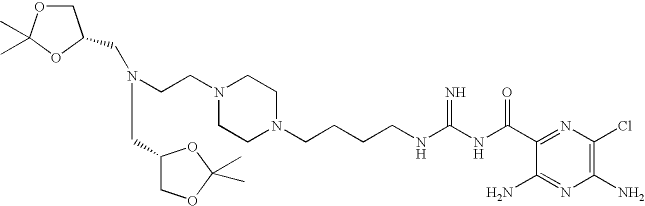

In another preferred embodiment, the compound of formula (III) is represented by the formula:

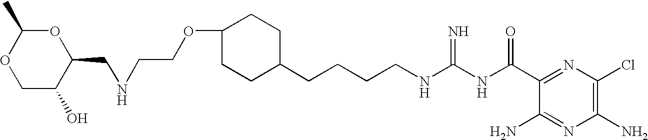

In another preferred embodiment, the compound of formula (III) is represented by the formula:

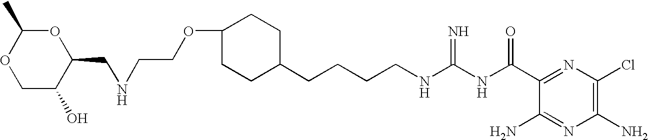

In another preferred embodiment, the compound of formula (III) is represented by the formula:

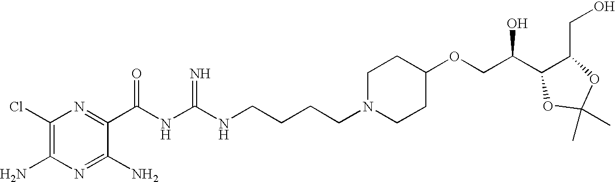

In another preferred embodiment, the compound of formula (III) is represented by the formula:

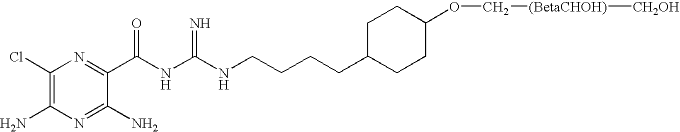

In another preferred embodiment, the compound of formula (III) is represented by the formula:

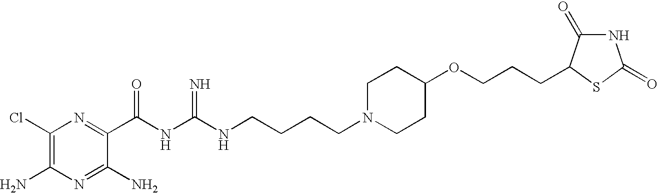

In another preferred embodiment, the compound of formula (III) is represented by the formula:

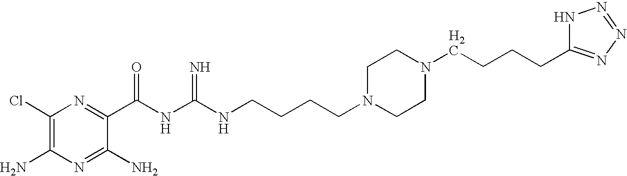

In another preferred embodiment, the compound of formula (III) is represented by the formula:

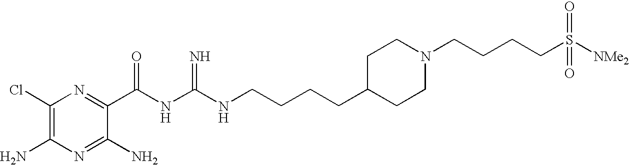

In another preferred embodiment, the compound of formula (III) is represented by the formula:

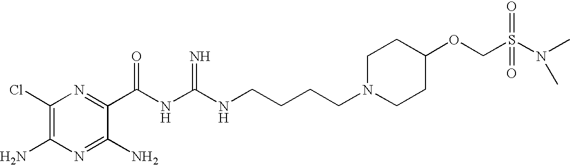

In another preferred embodiment, the compound of formula (III) is represented by the formula:

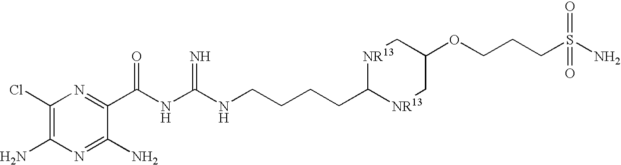

In another preferred embodiment, the compound of formula (III) is represented by the formula:

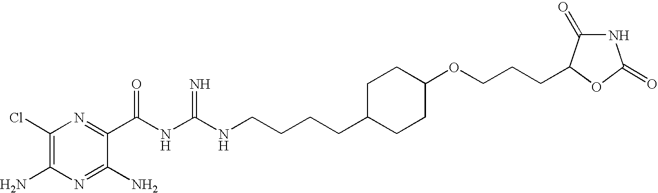

In another preferred embodiment, the compound of formula (III) is represented by the formula:

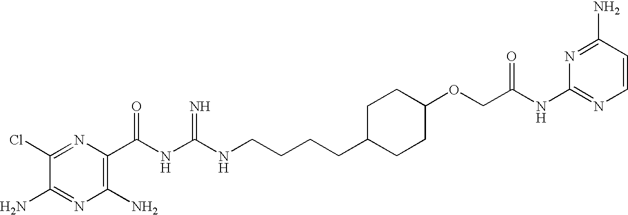

In another preferred embodiment, the compound of formula (III) is represented by the formula:

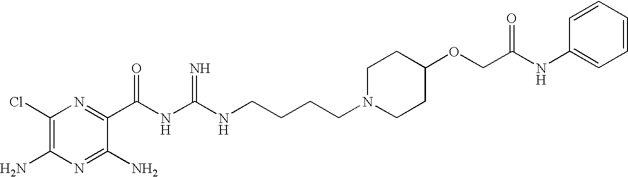

In another preferred embodiment, the compound of formula (III) is represented by the formula:

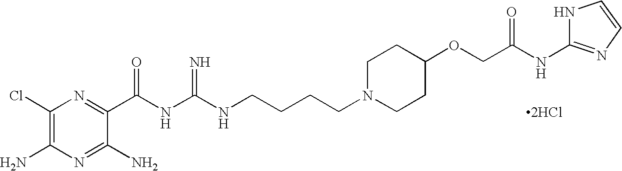

In another preferred embodiment, the compound of formula (III) is represented by the formula:

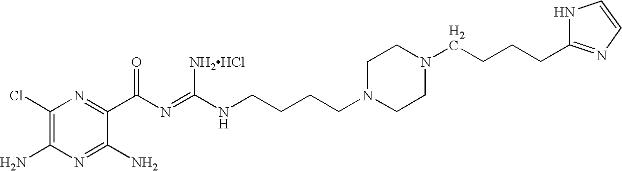

In another preferred embodiment, the compound of formula (III) is represented by the formula:

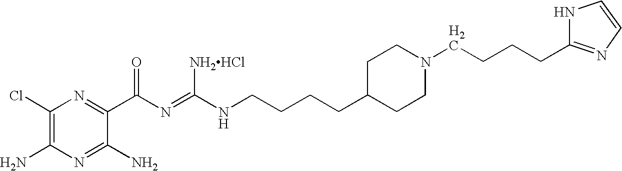

In another preferred embodiment, the compound of formula (III) is represented by the formula:

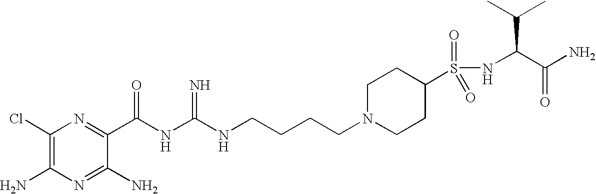

In another preferred embodiment, the compound of formula (III) is represented by the formula:

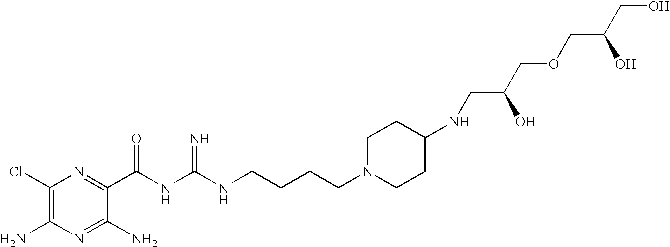

In another preferred embodiment, the compound of formula (III) is represented by the formula:

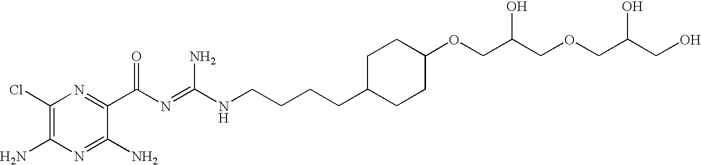

In another preferred embodiment, the compound of formula (III) is represented by the formula:

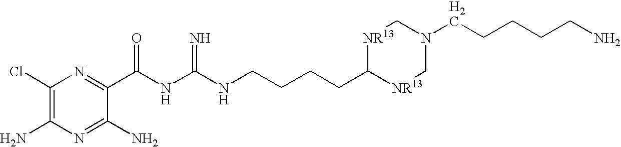

In another preferred embodiment, the compound of formula (III) is represented by the formula:

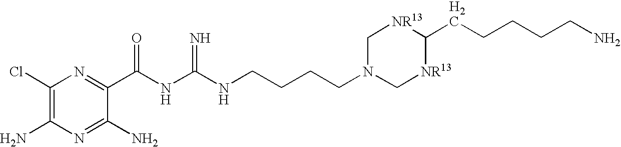

In another preferred embodiment, the compound of formula (III) is represented by the formula:

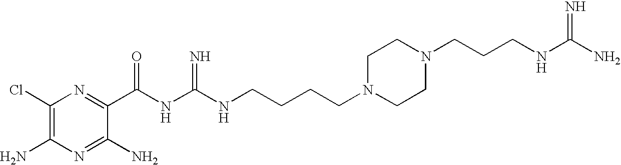

In another preferred embodiment, the compound of formula (III) is represented by the formula:

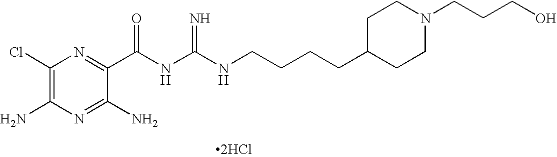

In another preferred embodiment, the compound of formula (III) is represented by the formula:

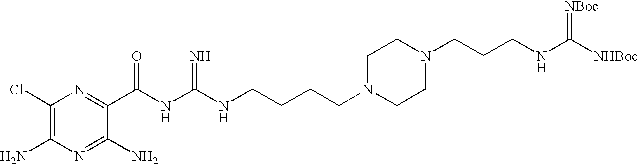

In another preferred embodiment, the compound of formula (III) is represented by the formula:

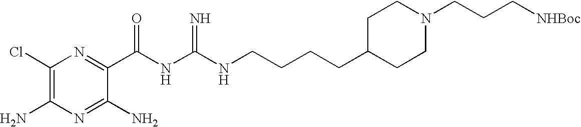

In another preferred embodiment, the compound of formula (III) is represented by the formula:

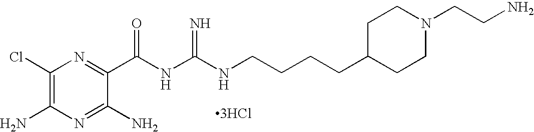

In another preferred embodiment, the compound of formula (III) is represented by the formula:

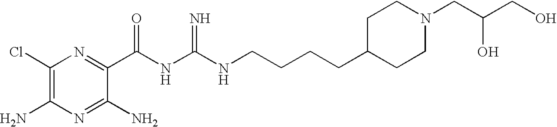

In another preferred embodiment, the compound of formula (III) is represented by the formula:

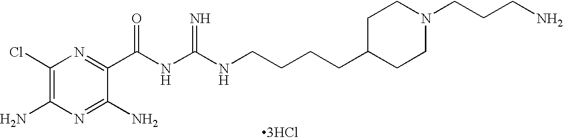

In another preferred embodiment, the compound of formula (III) is represented by the formula:

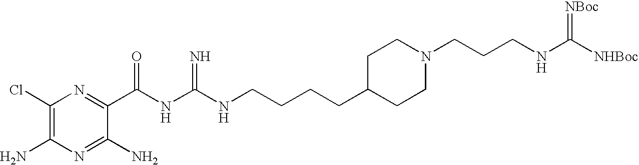

In another preferred embodiment, the compound of formula (III) is represented by the formula:

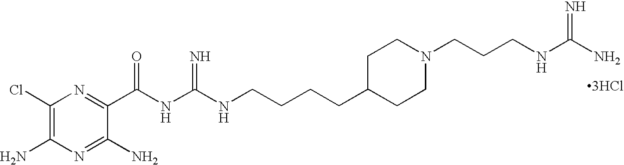

In another preferred embodiment, the compound of formula (III) is represented by the formula:

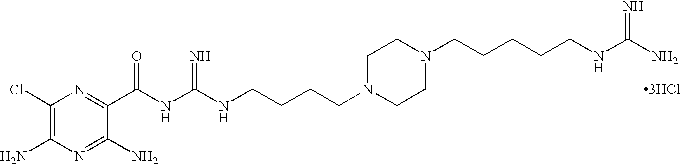

In another preferred embodiment, the compound of formula (III) is represented by the formula:

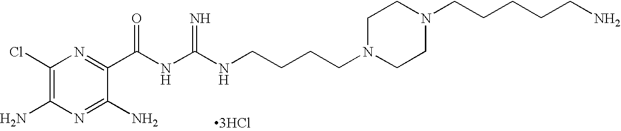

In another preferred embodiment, the compound of formula (III) is represented by the formula:

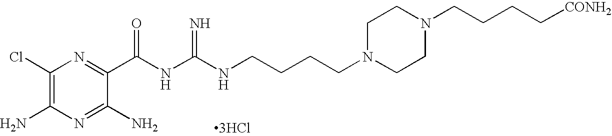

In another preferred embodiment, the compound of formula (III) is represented by the formula:

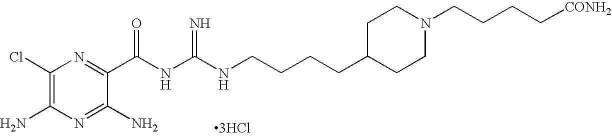

The active compounds disclosed herein may be administered to the lungs of a patient by any suitable means, but are preferably administered by administering an aerosol suspension of respirable particles comprised of the active compound, which the subject inhales. The compounds may be inhaled through the mouth or the nose. The active compound can be aerosolized in a variety of forms, such as, but not limited to, dry powder inhalants, metered dose inhalants or liquid/liquid suspensions. The quantity of sodium channel blocker included may be an amount sufficient to achieve the desired effect and as described in the attached applications.

Solid or liquid particulate sodium channel blocker prepared for practicing the present invention should include particles of respirable size: that is, particles of a size sufficiently small to pass through the mouth and larynx upon inhalation and into the bronchi and alveoli of the lungs. In general, particles ranging from about 1 to 5 microns in size (more particularly, less than about 4.7 microns in size) are respirable. Particles of non-respirable size which are included in the aerosol tend to be deposited in the throat and swallowed, and the quantity of non-respirable particles in the aerosol is preferably minimized. For nasal administration, a particle size in the range of 10-500 μm is preferred to ensure retention in the nasal cavity. Nasal administration may be useful where the pathogen typically enters through the nose. However, it is preferred to administer at least a portion of the sodium channel blocker in a dosage form which reaches the lungs to ensure effective prophylactic treatment in cases where the pathogen is expected to reach the lungs.

The dosage of active compound will vary depending on the prophylactic effect desired and the state of the subject, but generally may be an amount sufficient to achieve dissolved concentrations of active compound on the airway surfaces of the subject as described in the attached applications. Depending upon the solubility of the particular formulation of active compound administered, the daily dose may be divided among one or several unit dose administrations. The dosage may be provided as a prepackaged unit by any suitable means (e.g., encapsulating in a gelatin capsule).

Pharmaceutical formulations suitable for airway administration include formulations of solutions, emulsions, suspensions and extracts. See generally, J. Naim, Solutions, Emulsions, Suspensions and Extracts, in Remington: The Science and practice of Pharmacy, chap. 86 (19^{th} ed. 1995). Pharmaceutical formulations suitable for nasal administration may be prepared as described in U.S. Pat. No. 4,389,393 to Schor; U.S. Pat. No. 5,707,644 to Illum, U.S. Pat. No. 4,294,829 to Suzuki, and 4,835,142 to Suzuki.

In the manufacture of a formulation according to the invention, active agents or the physiologically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a capsule, which may contain from 0.5% to 99% by weight of the active compound. One or more active compounds may be incorporated in the formulations of the invention, which formulations may be prepared by any of the well-known techniques of pharmacy consisting essentially of admixing the components.

Aerosols or mists of liquid particles comprising the active compound may be produced by any suitable means, such as, for nasal administration, by a simple nasal spray with the active compound in an aqueous pharmaceutically acceptable carrier such as sterile saline solution or sterile water. Other means include producing aerosols with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers may consist of the active ingredient in a liquid carrier. The carrier is typically water (and most preferably sterile, pyrogen-free water) or a dilute aqueous alcoholic solution, preferably made isotonic with body fluids by the addition of, for example, sodium chloride.

Aerosols or mists of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. Such aerosol generators are known in the art. By way of example, see U.S. Pat. No. 5,725,842.

One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant.

A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 150 μl to produce a fine particle spray containing the active ingredient. Any propellant may be used in carrying out the present invention, including both chlorofluorocarbon-containing propellants and non-chlorofluorocarbon-containing propellants. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol, surfactants, such as oleic acid or sorbitan trioleate, antioxidants, preservatives such as methyl hydroxybenzoate, volatile oils, buffering agents and suitable flavoring agents.

Compositions containing respirable dry particles of sodium channel blockers as described in the attached applications may be prepared as detailed in those applications. The active compound may be formulated alone (i.e., the solid particulate composition may consist essentially of the active compound) or in combination with a dispersant, diluent or carrier, such as sugars (i.e., lactose, sucrose, trehalose, mannitol) or other acceptable excipients for lung or airway delivery, which may be blended with the active compound in any suitable ratio (e.g., a 1 to 1 ratio by weight). The dry powder solid particulate compound may be obtained by methods known in the art, such as spray-drying, milling, freeze-drying, and the like.

The aerosol or mist, whether formed from solid or liquid particles, may be produced by the aerosol generator at a rate of from about 10 to about 150 liters per minute, more preferably from about 30 to about 150 liters per minute, and most preferably about 60 liters per minute. Aerosols containing greater amounts of medicament may be administered more rapidly.

Other medicaments may be administered with the active compounds disclosed if such medicament is compatible with the active compound and other ingredients in the formulation and can be administered as described herein.

The pathogens which may be protected against by the prophylactic post exposure, rescue and therapeutic treatment methods of the invention include any pathogens which may enter the body through the mouth, nose or nasal airways, thus proceeding into the lungs. Typically, the pathogens will be airborne pathogens, either naturally occurring or by aerosolization. The pathogens may be naturally occurring or may have been introduced into the environment intentionally after aerosolization or other method of introducing the pathogens into the environment. Many pathogens which are not naturally transmitted in the air have been or may be aerosolized for use in bioterrorism.

The pathogens for which the treatment of the invention may be useful includes, but is not limited to, category A, B and C priority pathogens as set forth by the NIAID. These categories correspond generally to the lists compiled by the Centers for Disease Control and Prevention (CDC). As set up by the CDC, Category A agents are those that can be easily disseminated or transmitted person-to-person, cause high mortality, with potential for major public health impact. Category B agents are next in priority and include those that are moderately easy to disseminate and cause moderate morbidity and low mortality. Category C consists of emerging pathogens that could be engineered for mass dissemination in the future because of their availability, ease of production and dissemination and potential for high morbidity and mortality.

Category A: Bacillus anthracis (anthrax),
Clostridium botulinum (botulism),
Yersinia pestis (plague),
Variola major (smallpox) and other pox viruses,
Francisella tularensis (tularemia),
Viral hemorrhagic fevers
Arenaviruses,
   LCM (lymphocytic choriomeningitis), Junin virus,
Machupo virus, Guanarite virus,
   Lassa Fever,
Bunyaviruses,
   Hantavirus,
   Rift Valley Fever,
Flaviviruses,
   Dengue,
Filoviruses,
   Ebola
   Marburg;

Category B: Burkholderia pseudomallei (melioidosis),
Coxiella burnetii (Q fever),
Brucella species (brucellosis),
Burkholderia mallei (glanders),
Ricin toxin from Ricinus communis,
Epsilon toxin of Clostridium perfringens,
Staphylococcal enterotoxin B,
Typhus fever (Rickettsia prowazekii),
Food and water-borne pathogens
   bacteria:
      Diarrheagenic Escherichia coli,
      Pathogenic vibrios,
      Shigella species,
      Salmonella species,
      Listeria monocytogenes,
      campylobacter jejuni,
      Yersinia enterocolitica;
   Viruses
      Caliciviruses,
      Hepatitis A;
   Protozoa
      Cryptosporidium parvum,
      Cyclospora cayatenensis,
      Giardia lamblia,
      Entamoeba histolytica,
      Toxoplasma,
      Microsporidia, and
   Additional viral encephalitides
      West Nile virus,
      LaCrosse,
      California encephalitis,
      Venezuelan equine encephalitis,
      Eastern equine encephalitis,
      Western equine encephalitis,
      Japanese encephalitis virus and
      Kyasanur forest virus, and

Category C: emerging infectious disease threats such as Nipah virus and additional hantaviruses, tickborne hemorrhagic fever viruses such as Crimean Congo hemorrhagic fever virus, tickborne encephalitis viruses, yellow fever, multi-drug resistant tuberculosis, influenza, other rickettsias and rabies.

Additional pathogens which may be protected against or the infection risk therefrom reduced include influenza viruses, rhinoviruses, adenoviruses and respiratory syncytial viruses, and the like. A further pathogen which may be protected against is the coronavirus which is believed to cause severe acute respiratory syndrome (SARS).

A number of the above-listed pathogens are known to be particularly harmful when introduced into the body through the air. For example, Bacillus anthracis, the agent which causes anthrax, has three major clinical forms, cutaneous, inhalational, and gastrointestinal. All three forms may lead to death but early antibiotic treatment of cutaneous and gastrointestinal anthrax usually cures those forms of anthrax. Inhalational anthrax, on the other hand, is a potentially fatal disease even with antibiotic treatment. Initial symptoms may resemble a common cold. After several days, the symptoms may progress to severe breathing problems and shock. For naturally occurring or accidental infections, even with appropriate antibiotics and all other available supportive care, the historical fatality rate is believed to be about 75 percent, according to the NIAID. Inhalational anthrax develops after spores are deposited in alveolar spaces and subsequently ingested by pulmonary alveolar macrophages. Surviving spores are then transported to the mediastinal lymph nodes, where they may germinate up to 60 days or longer. After germination, replicating bacteria release toxins that result in disease. This process is interrupted by administration of a prophylactically effective amount of a sodium channel blocker, as the spores may be wholly or partially eliminated from the body by removal of lung mucous secretions hydrated through the action of the sodium channel blocker.

Another pathogen of primary concern as one of the most dangerous potential biological weapons because it is easily transmitted from person to person, no effective therapy exists and few people carry full immunity to the virus, is the small pox virus, Variola major. Smallpox spreads directly from person to person, primarily by aerosolized saliva droplets expelled from an infected person. Initial symptoms include high fever, fatigue, headache and backache followed in two or three days by a characteristic rash.

An embodiment of the present invention provides a method of prophylactically treating one or more individuals exposed or potentially exposed to smallpox virus or other pox virus comprising the administration of a prophylactically effective amount of a sodium channel blocker. The administration of an effective amount of a sodium channel blocker will function to allow the Variola major virus or other pox virus present in the aerosolized saliva droplets to which the individual was exposed to be wholly or partially removed from the body by removal of hydrated lung mucous secretions hydrated through the action of the sodium channel blocker.

The bacterium Yersinia pestis causes plague and is widely available throughout the world. NIAID has reported that infection by inhalation of even small numbers of virulent aerosolized Y. pestis bacilli can lead to pneumonic plague, which has a mortality rate of almost 100% if left untreated. Pneumonic plague has initial symptoms of fever and cough which resemble other respiratory illnesses. Antibiotics are effective against plague but success with antibiotics depends on how quickly drug therapy is started, the dose of inhaled bacteria and the level of supportive care for the patient; an effective vaccine is not widely available.

An embodiment of the present invention provides a method of prophylactically treating one or more individuals exposed or potentially exposed to aerosolized Y. pestis bacilli comprising the administration of a sodium channel blocker. The administration of an effective amount of a sodium channel blocker will function to allow the aerosolized Y. pestis bacilli to be wholly or partially removed from the body by removal of hydrated lung mucous secretions hydrated through the action of the sodium channel blocker.

Botulinum toxin is another substance believed to present a major bioterrorism threat as it is easily released into the environment. Antibiotics are not effective against botulinum toxin and no approved vaccine exists. Although the toxin may be transmitted through food, the botulinum toxin is absorbed across mucosal surfaces and, thus, embodiments of the present invention provide a method of prophylactically treating one or more individuals exposed or potentially exposed to botulinum toxin comprising the administration of a sodium channel blocker.

The NIAID has identified the bacteria that causes tularemia as a potential bioterrorist agent because Francisella tularensis is capable of causing infection with as few as ten organisms and due to its ability to be aerosolized. Natural infection occurs after inhalation of airborne particles. Tularemia may be treated with antibiotics and an experimental vaccine exists but knowledge of optimal therapeutic approaches for tularemia is limited because very few investigators are working on this disease. An embodiment of the present invention provides a method of prophylactically treating one or more individuals exposed or potentially exposed to aerosolized Francisella tularensis comprising the administration of a sodium channel blocker. The administration of an effective amount of a sodium channel blocker will function to allow the aerosolized Francisella tularensis to be wholly or partially removed from the body by removal of hydrated lung mucous secretions hydrated through the action of the sodium channel blocker.

The Category B and C bacteria most widely believed to have the potential to infect by the aerosol route include gram negative bacteria such as Brucella species, Burkholderia pseudomallei, Burkholderia mallei, Coxiella burnetii, and select Rickettsia spp. Each of these agents is believed to be capable of causing infections following inhalation of small numbers of organisms. Brucella spp. may cause brucellosis. Four of the six Brucella spp., B. suis, B. melitensis, B. abortus and B. canis, are known to cause brucellosis in humans. Burkholderia pseudomallei may cause melioidosis in humans and other mammals and birds. Burkholderia mallei, is the organism that causes glanders, normally a disease of horses, mules and donkeys but infection following aerosol exposure has been reported, according to NIAID. Coxiella burnetii, may cause Q fever and is highly infectious. Infections have been reported through aerosolized bacteria and inhalation of only a few organisms can cause infections. R. prowazekii, R. rickettsii, R. conorrii and R. typhi have been found to have low-dose infectivity via the aerosol route.

Methods are provided of prophylactically treating one or more individuals exposed or potentially exposed to aerosolized gram negative bacteria such as Brucella species, Burkholderia pseudomallei, Burkholderia mallei, Coxiella burnetii, and select Rickettsia spp comprising the administration of a sodium channel blocker. The administration of an effective amount of a sodium channel blocker will function to allow the aerosolized gram negative bacteria to be wholly or partially removed from the body by removal of hydrated lung mucous secretions hydrated through the action of the sodium channel blocker.

A number of typically arthropod-borne viruses are believed to pose a significant threat as potential bioterrorist weapons due to their extreme infectivity following aerosolized exposure. These viruses include arboviruses which are important agents of viral encephalitides and hemorrhagic fevers. Such viruses may include alphaviruses such as Venezuelan equine encephalitis virus, eastern equine encephalitis virus and western equine encephalitis virus. Other such viruses may include flaviviruses such as West Nile virus, Japanese encephalitis virus, Kyasanur forest disease virus, tick-borne encephalitis virus complex and yellow fever virus. An additional group of viruses which may pose a threat include bunyaviruses such as California encephalitis virus, or La Crosse virus, Crimean-Congo hemorrhagic fever virus. According to the NIAID, vaccines or effective specific therapeutics are available for only a very few of these viruses. In humans, arbovirus infection is usually initially asymptomatic or causes nonspecific flu-like symptoms such as fever, aches and fatigue.

An embodiment of the present invention provides a method of prophylactically treating one or more individuals exposed or potentially exposed to aerosolized arboviruses comprising the administration of a sodium channel blocker. The administration of an effective amount of a sodium channel blocker will function to allow the arboviruses to be wholly or partially removed from the body by removal of hydrated lung mucous secretions hydrated through the action of the sodium channel blocker.

Certain category B toxins such as ricin toxin from Ricinus communis, epsilon toxin of Clostridium perfringens and Staphylococcal enterotoxin B, also are viewed as potential bioterrorism tools. Each of these toxins may be delivered to the environment or population by inhalational exposure to aerosols. Low dose inhalation of ricin toxin may cause nose and throat congestion and bronchial asthma while higher dose inhalational exposure caused severe pneumonia, acute inflammation and diffuse necrosis of the airways in nonhuman primates. Clostridium perfringens is an anaerobic bacterium that can infect humans and animals. Five types of bacteria exist that produce four major lethal toxins and seven minor toxins, including alpha toxin, associated with gas gangrene, beta toxin, responsible for necrotizing enteritis, and epsilon toxin, a neurotoxin that leads to hemorrhagic enteritis in goats and sheep. Inhalation of Staphylococcus aureus has resulted in extremely high fever, difficulty breathing, chest pain and headache.

An embodiment of the present invention provides a method of prophylactically treating one or more individuals exposed or potentially exposed to aerosolized toxins comprising the administration of a sodium channel blocker. The administration of an effective amount of a sodium channel blocker will function to allow the aerosolized toxins to be wholly or partially removed from the body by removal of hydrated lung mucous secretions hydrated through the action of the sodium channel blocker.

Mycobacterium tuberculosis bacteria causes tuberculosis and is spread by airborne droplets expelled from the lungs when a person with tuberculosis coughs, sneezes or speaks. An embodiment of the present invention provides a method of prophylactically treating one or more individuals exposed or potentially exposed to Mycobacterium tuberculosis bacteria comprising the administration of a sodium channel blocker. The administration of an effective amount of a sodium channel blocker will function to allow the Mycobacterium tuberculosis bacteria to be wholly or partially removed from the body by removal of hydrated lung mucous secretions hydrated through the action of the sodium channel blocker.

The methods disclosed may also be used against more common pathogens such as influenza viruses, rhinoviruses, adenoviruses and respiratory syncytial viruses (RSV). An embodiment of the present invention provides a method of prophylactically or therapeutically treating one or more individuals exposed or potentially exposed to one of these viruses comprising the administration of a sodium channel blocker. The administration of an effective amount of a sodium channel blocker will function to allow the virus to be wholly or partially removed from the body by removal of hydrated lung mucous secretions hydrated through the action of the sodium channel blocker.

The methods of the present invention may further be used against the virus believed to be responsible for SARS, the coronavirus. Severe acute respiratory syndrome is a respiratory illness that is believed to spread by person-to-person contact, including when someone coughs or sneezes droplets containing the virus onto others or nearby surfaces. The CDC currently believes that it is possible that SARS can be spread more broadly through the air or by other ways that are not currently known. Typically, SARS begins with a fever greater than 100.4° F. Other symptoms include headache and body aches. After two to seven days, SARS patients may develop a dry cough and have trouble breathing.

To the extent SARS is caused by an airborne pathogen, the present invention provides a method of prophylactically treating one or more individuals exposed or potentially exposed to the SARS virus comprising the administration of a sodium channel blocker. The administration of an effective amount of a sodium channel blocker will function to allow the virus to be wholly or partially removed from the body by removal of hydrated lung mucous secretions hydrated through the action of the sodium channel blocker.

The compounds of formulas (I), (II) and (III) may be synthesized according to procedures known in the art. A representative synthetic procedure is shown in the scheme below:

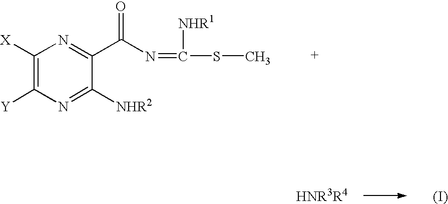

These procedures are described in, for example, E. J. Cragoe, “The Synthesis of Amiloride and Its Analogs” (Chapter 3) in Amiloride and Its Analogs, pp. 25-36, incorporated herein by reference. Other methods of preparing the compounds are described in, for example, U.S. 3,313,813, incorporated herein by reference. See in particular Methods A, B, C, and D described in U.S. Pat. No. 3,313,813. Other methods useful for the preparation of these compounds, are described in, for example, U.S. Provisional Applications 60/495,725, filed Aug. 19, 2003, 60/495,712, filed Aug. 19, 2003 and 60/495,720, filed Aug. 19, 2003, incorporated herein by reference. Several assays may be used to characterize the compounds of the present invention. Representative assays are discussed below.

In Vitro Measure of Sodium Channel Blocking Activity and Reversibility

One assay used to assess mechanism of action and/or potency of the compounds of the present invention involves the determination of lumenal drug inhibition of airway epithelial sodium currents measured under short circuit current (I_{SC}) using airway epithelial monolayers mounted in Ussing chambers. Cells obtained from freshly excised human, dog, sheep or rodent airways are seeded onto porous 0.4 micron Snapwell™ Inserts (CoStar), cultured at air-liquid interface (ALI) conditions in hormonally defined media, and assayed for sodium transport activity (I_{SC}) while bathed in Krebs Bicarbonate Ringer (KBR) in Using chambers. All test drug additions are to the lumenal bath with half-log dose addition protocols (from 1×10⁻¹¹ M to 3×10⁻⁵ M), and the cumulative change in I_{SC} (inhibition) recorded. All drugs are prepared in dimethyl sulfoxide as stock solutions at a concentration of 1×10⁻² M and stored at −20° C. Eight preparations are typically run in parallel; two preparations per run incorporate amiloride and/or benzamil as positive controls. After the maximal concentration (5×10⁻⁵ M) is administered, the lumenal bath is exchanged three times with fresh drug-free KBR solution, and the resultant I_{SC} measured after each wash for approximately 5 minutes in duration. Reversibility is defined as the percent return to the baseline value for sodium current after the third wash. All data from the voltage clamps are collected via a computer interface and analyzed off-line.

Dose-effect relationships for all compounds are considered and analyzed by the Prism 3.0 program. IC₅₀ values, maximal effective concentrations, and reversibility are calculated and compared to amiloride and benzamil as positive controls.

Pharmacological Assays of Absorption

(1) Apical Disappearance Assay

Bronchial cells (dog, human, sheep, or rodent cells) are seeded at a density of 0.25'10⁶/cm² on a porous Transwell-Col collagen-coated membrane with a growth area of 1.13 cm² grown at an air-liquid interface in hormonally defined media that promotes a polarized epithelium. From 12 to 20 days after development of an air-liquid interface (ALI) the cultures are expected to be >90% ciliated, and mucins will accumulate on the cells. To ensure the integrity of primary airway epithelial cell preparations, the transepithelial resistance (Rₜ) and transepithelial potential differences (PD), which are indicators of the integrity of polarized nature of the culture, are measured. Human cell systems are preferred for studies of rates of absorption from apical surfaces. The disappearance assay is conducted under conditions that mimic the “thin” films in vivo (˜25 μl) and is initiated by adding experimental sodium channel blockers or positive controls (amiloride, benzamil, phenamil) to the apical surface at an initial concentration of 10 μM. A series of samples (5 μl volume per sample) is collected at various time points, including 0, 5, 20, 40, 90 and 240 minutes. Concentrations are determined by measuring intrinsic fluorescence of each sodium channel blocker using a Fluorocount Microplate Flourometer or HPLC. Quantitative analysis employs a standard curve generated from authentic reference standard materials of known concentration and purity. Data analysis of the rate of disappearance is performed using nonlinear regression, one phase exponential decay (Prism V 3.0).

2. Confocal Microscopy Assay of Amiloride Congener Uptake

Virtually all amiloride-like molecules fluoresce in the ultraviolet range. This property of these molecules may be used to directly measure cellular update using x-z confocal microscopy. Equimolar concentrations of experimental compounds and positive controls including amiloride and compounds that demonstrate rapid uptake into the cellular compartment (benzamil and phenamil) are placed on the apical surface of airway cultures on the stage of the confocal microscope. Serial x-z images are obtained with time and the magnitude of fluorescence accumulating in the cellular compartment is quantitated and plotted as a change in fluorescence versus time.

3. In vitro Assays of Compound Metabolism

Airway epithelial cells have the capacity to metabolize drugs during the process of transepithelial absorption. Further, although less likely, it is possible that drugs can be metabolized on airway epithelial surfaces by specific ectoenzyme activities. Perhaps more likely as an ecto-surface event, compounds may be metabolized by the infected secretions that occupy the airway lumens of patients with lung disease, e.g. cystic fibrosis. Thus, a series of assays is performed to characterize the compound metabolism that results from the interaction of test compounds with human airway epithelia and/or human airway epithelial lumenal products.

In the first series of assays, the interaction of test compounds in KBR as an “ASL” stimulant are applied to the apical surface of human airway epithelial cells grown in the T-Col insert system. For most compounds, metabolism (generation of new species) is tested for using high performance liquid chromatography (HPLC) to resolve chemical species and the endogenous fluorescence properties of these compounds to estimate the relative quantities of test compound and novel metabolites. For a typical assay, a test solution (25 μl KBR, containing 10 μM test compound) is placed on the epithelial lumenal surface. Sequential 5 to 10 μl samples are obtained from the lumenal and serosal compartments for HPLC analysis of (1) the mass of test compound permeating from the lumenal to serosal bath and (2) the potential formation of metabolites from the parent compound. In instances where the fluorescence properties of the test molecule are not adequate for such characterizations, radiolabeled compounds are used for these assays. From the HPLC data, the rate of disappearance and/or formation of novel metabolite compounds on the lumenal surface and the appearance of test compound and/or novel metabolite in the basolateral solution is quantitated. The data relating the chromatographic mobility of potential novel metabolites with reference to the parent compound are also quantitated.

To analyze the potential metabolism of test compounds by CF sputum, a “representative” mixture of expectorated CF sputum obtained from 10 CF patients (under IRB approval) has been collected. The sputum has been be solubilized in a 1:5 mixture of KBR solution with vigorous vortexing, following which the mixture was split into a “neat” sputum aliquot and an aliquot subjected to ultracentrifugation so that a “supernatant” aliquot was obtained (neat=cellular; supernatant=liquid phase). Typical studies of compound metabolism by CF sputum involve the addition of known masses of test compound to “neat” CF sputum and aliquots of CF sputum “supernatant” incubated at 37° C., followed by sequential sampling of aliquots from each sputum type for characterization of compound stability/metabolism by HPLC analysis as described above. As above, analysis of compound disappearance, rates of formation of novel metabolites, and HPLC mobilities of novel metabolites are then performed.

4. Pharmacological Effects and Mechanism of Action of the Drug in Animals

The effect of compounds for enhancing mucociliary clearance (MCC) can be measured using an in vivo model described by Sabater et al., Journal of Applied Physiology, 1999, pp. 2191-2196, incorporated herein by reference.

Animal Preparation: Adult ewes (ranging in weight from 25 to 35 kg) were restrained in an upright position in a specialized body harness adapted to a modified shopping cart. The animals' heads were immobilized and local anesthesia of the nasal passage was induced with 2% lidocaine. The animals were then nasally intubated with a 7.5 mm internal diameter endotracheal tube (ETT). The cuff of the ETT was placed just below the vocal cords and its position was verified with a flexible bronchoscope. After intubation the animals were allowed to equilibrate for approximately 20 minutes prior to initiating measurements of mucociliary clearance.

Administration of Radio-aerosol: Aerosols of ^{99m}Tc-Human serum albumin (3.1 mg/ml; containing approximately 20 mCi) were generated using a Raindrop Nebulizer which produces a droplet with a median aerodynamic diameter of 3.6 μm. The nebulizer was connected to a dosimetry system consisting of a solenoid valve and a source of compressed air (20 psi). The output of the nebulizer was directed into a plastic T connector; one end of which was connected to the endotracheal tube, the other was connected to a piston respirator. The system was activated for one second at the onset of the respirator's inspiratory cycle. The respirator was set at a tidal volume of 500 mL, an inspiratory to expiratory ratio of 1:1, and at a rate of 20 breaths per minute to maximize the central airway deposition. The sheep breathed the radio-labeled aerosol for 5 minutes. A gamma camera was used to measure the clearance of ^{99m}Tc-Human serum albumin from the airways. The camera was positioned above the animal's back with the sheep in a natural upright position supported in a cart so that the field of image was perpendicular to the animal's spinal cord. External radio-labeled markers were placed on the sheep to ensure proper alignment under the gamma camera. All images were stored in a computer integrated with the gamma camera. A region of interest was traced over the image corresponding to the right lung of the sheep and the counts were recorded. The counts were corrected for decay and expressed as percentage of radioactivity present in the initial baseline image. The left lung was excluded from the analysis because its outlines are superimposed over the stomach and counts can be swallowed and enter the stomach as radio-labeled mucus.

Treatment Protocol (Assessment of activity at t-zero): A baseline deposition image was obtained immediately after radio-aerosol administration. At time zero, after acquisition of the baseline image, vehicle control (distilled water), positive control (amiloride), or experimental compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were extubated immediately following delivery of the total dose in order to prevent false elevations in counts caused by aspiration of excess radio-tracer from the ETT. Serial images of the lung were obtained at 15-minute intervals during the first 2 hours after dosing and hourly for the next 6 hours after dosing for a total observation period of 8 hours. A washout period of at least 7 days separated dosing sessions with different experimental agents.

Treatment Protocol (Assessment of Activity at t-4 hours): The following variation of the standard protocol was used to assess the durability of response following a single exposure to vehicle control (distilled water), positive control compounds (amiloride or benzamil), or investigational agents. At time zero, vehicle control (distilled water), positive control (amiloride), or investigational compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were restrained in an upright position in a specialized body harness for 4 hours. At the end of the 4-hour period animals received a single dose of aerosolized ^{99m}Tc-Human serum albumin (3.1 mg/ml; containing approximately 20 mCi) from a Raindrop Nebulizer. Animals were extubated immediately following delivery of the total dose of radio-tracer. A baseline deposition image was obtained immediately after radio-aerosol administration. Serial images of the lung were obtained at 15-minute intervals during the first 2 hours after administration of the radio-tracer (representing hours 4 through 6 after drug administration) and hourly for the next 2 hours after dosing for a total observation period of 4 hours. A washout period of at least 7 days separated dosing sessions with different experimental agents.

Statistics: Data were analyzed using SYSTAT for Windows, version 5. Data were analyzed using a two-way repeated ANOVA (to assess overall effects), followed by a paired t-test to identify differences between specific pairs. Significance was accepted when P was less than or equal to 0.05. Slope values (calculated from data collected during the initial 45 minutes after dosing in the t-zero assessment) for mean MCC curves were calculated using linear least square regression to assess differences in the initial rates during the rapid clearance phase.

### EXAMPLES

Having generally described this invention, a further understanding can be obtained by reference to certain specific examples which are provided herein for purposes of illustration only and are not intended to be limiting unless otherwise specified.

### Preparation of Sodium Channel Blockers

Materials and methods. All reagents and solvents were purchased from Aldrich Chemical Corp. and used without further purification. NMR spectra were obtained on either a Bruker WM 360 (¹H NMR at 360 MHz and ¹³C NMR at 90 MHz) or a Bruker AC 300 (¹H NMR at 300 MHz and ¹³C NMR at 75 MHz). Flash chromatography was performed on a Flash Elute system from Elution Solution (PO Box 5147, Charlottesville, Va. 22905) charged with a 90 g silica gel cartridge (40 M FSO-0110-040155, 32-63 μm) at 20 psi (N₂). GC-analysis was performed on a Shimadzu GC-17 equipped with a Heliflex Capillary Colunm (Alltech); Phase: AT-1, Length: 10 meters, ID: 0.53 mm, Film: 0.25 micrometers. GC Parameters: Injector at 320° C., Detector at 320° C., FID gas flow: H₂ at 40 ml/min., Air at 400 ml/min. Carrier gas: Split Ratio 16:1, N₂ flow at 15 ml/min., N₂ velocity at 18 cm/sec. The temperature program is 70° C. for 0-3 min, 70-300° C. from 3-10 min, 300° C. from 10-15 min.

HPLC analysis was performed on a Gilson 322 Pump, detector UV/Vis-156 at 360 nm, equipped with a Microsorb MV C8 column, 100 A, 25 cm. Mobile phase: A=acetonitrile with 0.1% TFA, B=water with 0.1% TFA. Gradient program: 95:5 B:A for 1 min, then to 20:80 B:A over 7 min, then to 100% A over 1 min, followed by washout with 100% A for 11 min, flow rate: 1 ml/min.

The following examples depict the synthesis of compounds according to Formula I.

### FORMULA I EXAMPLES

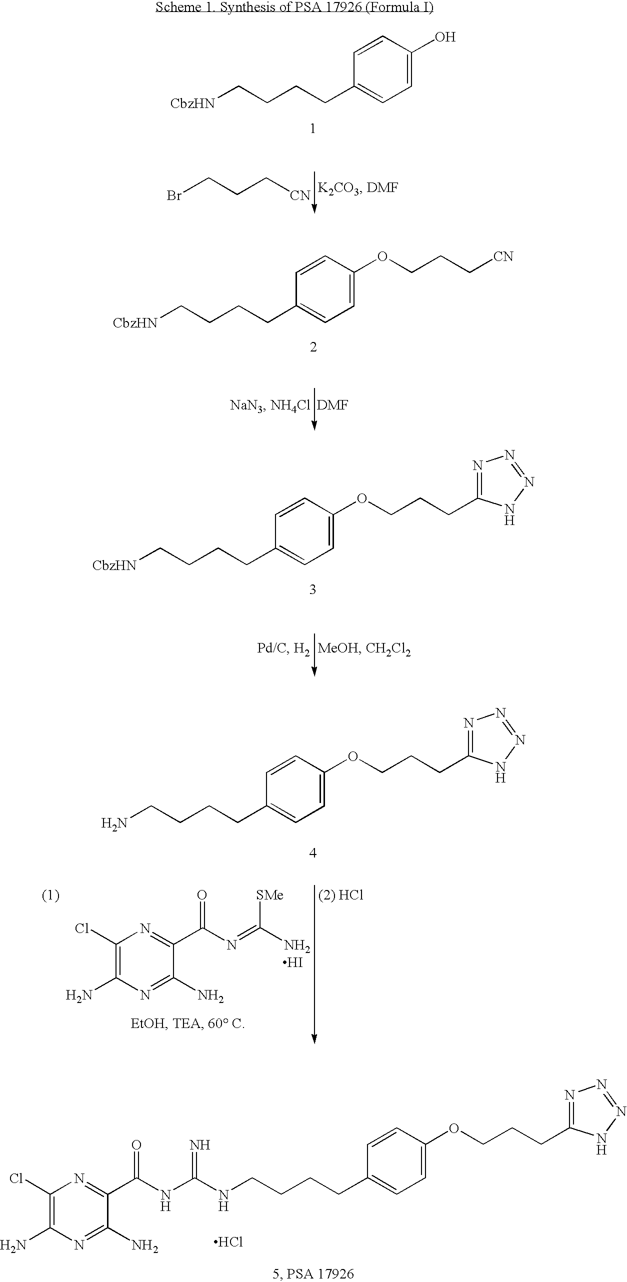

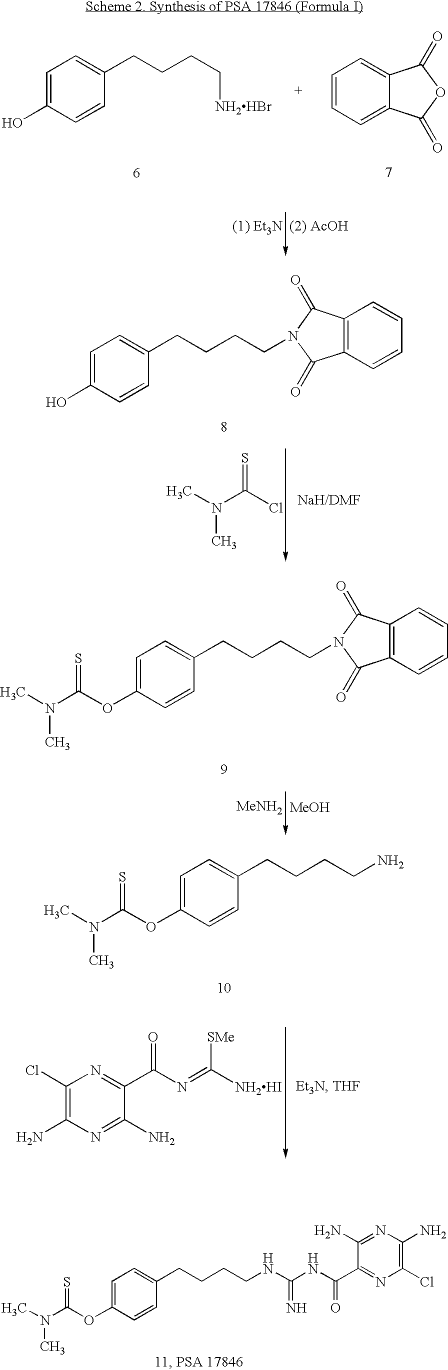

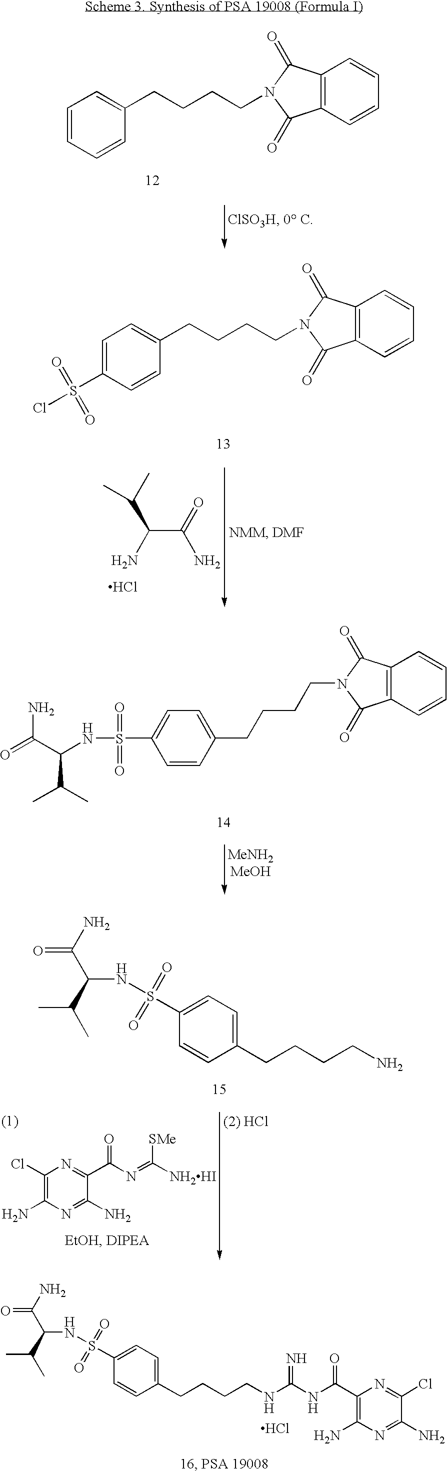

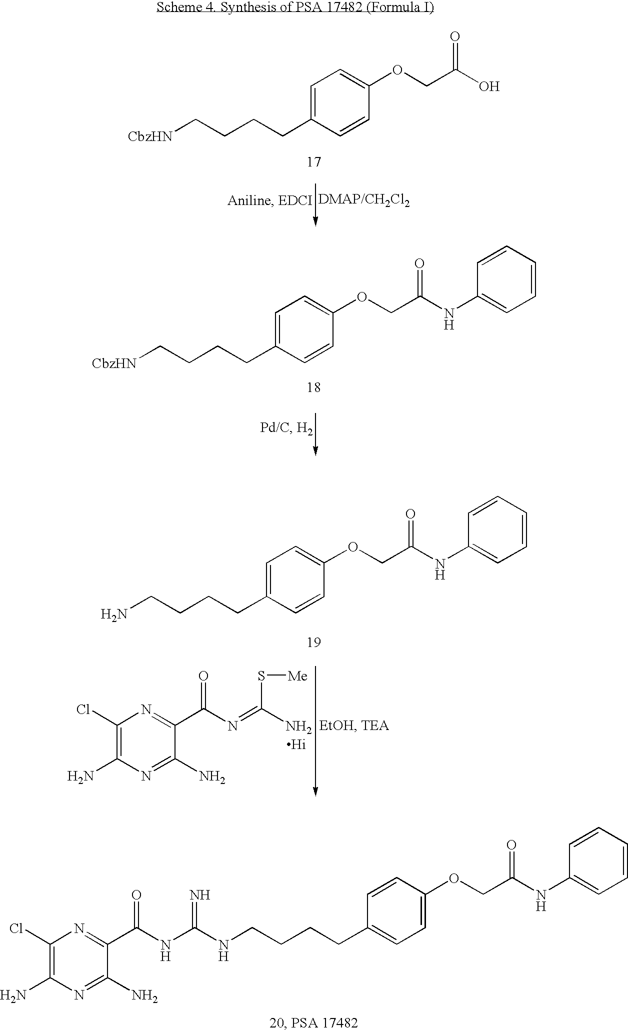

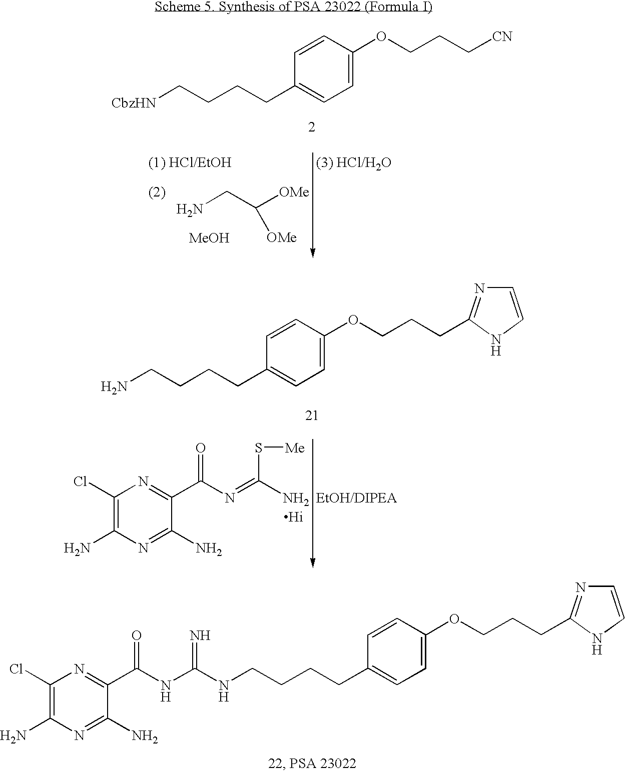

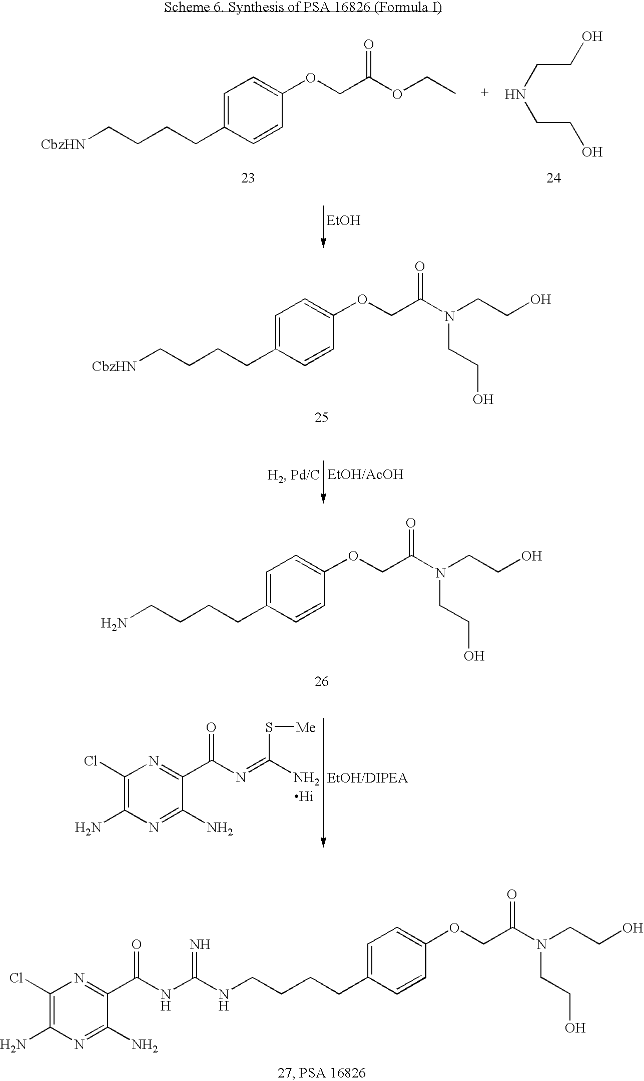

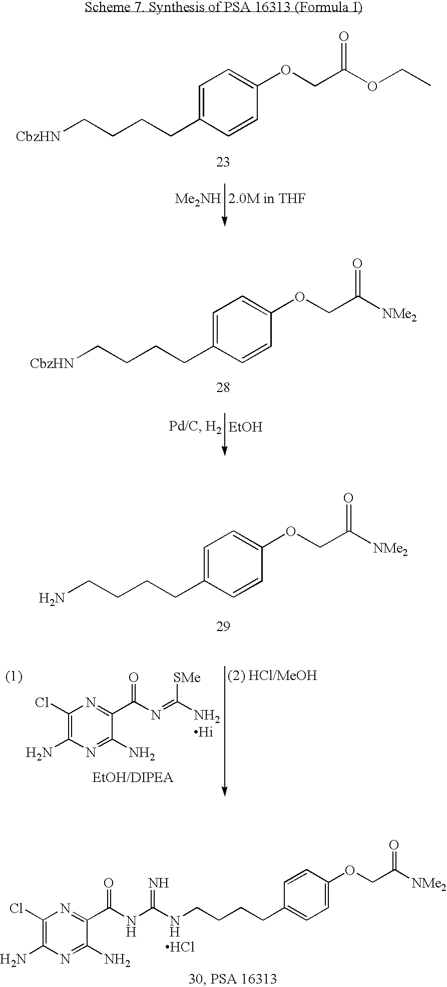

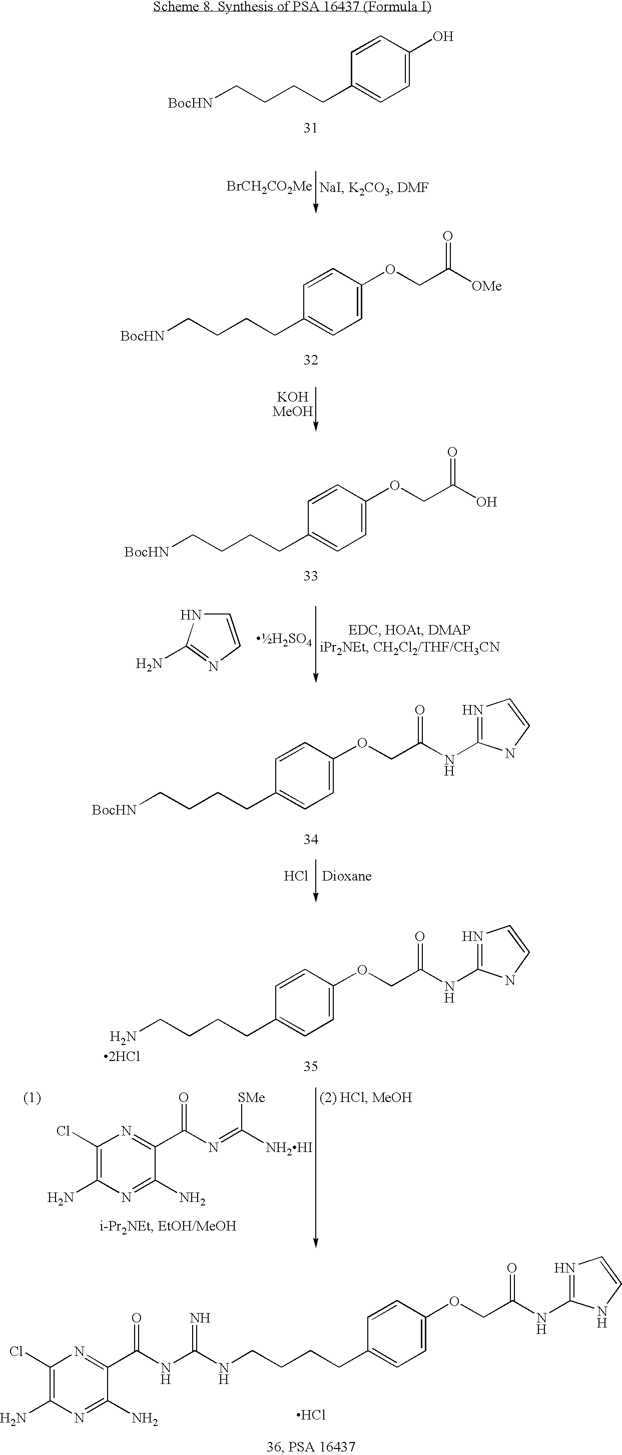

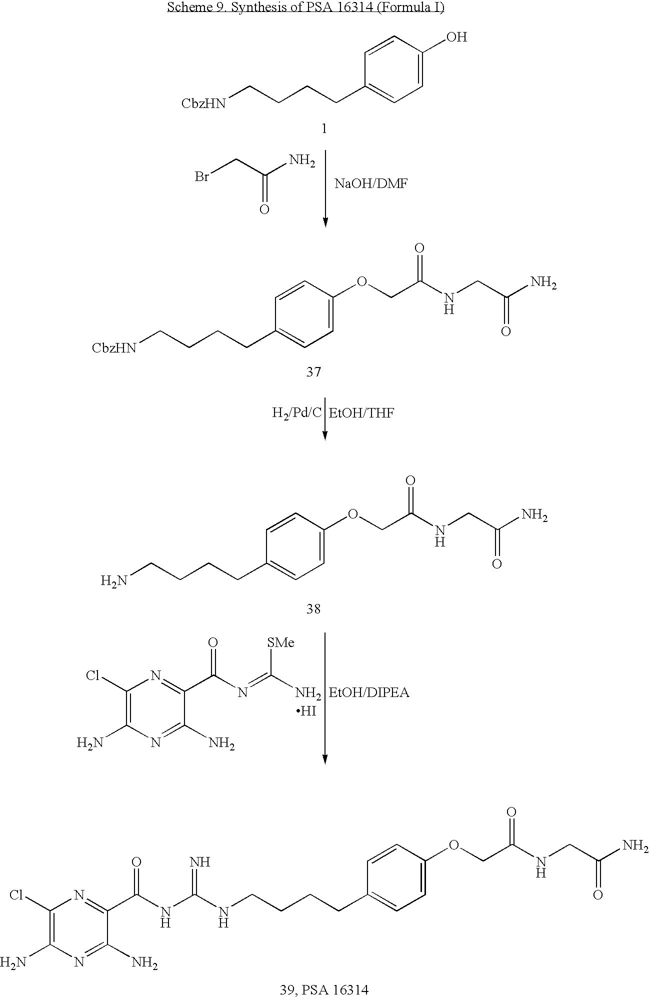

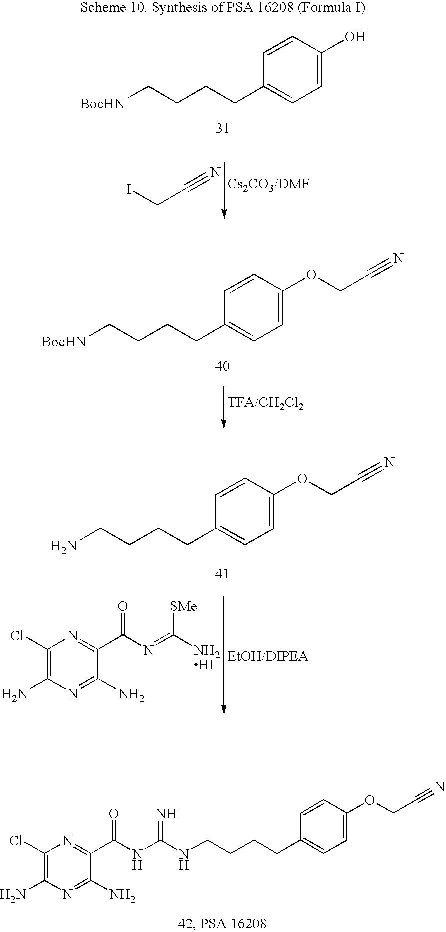

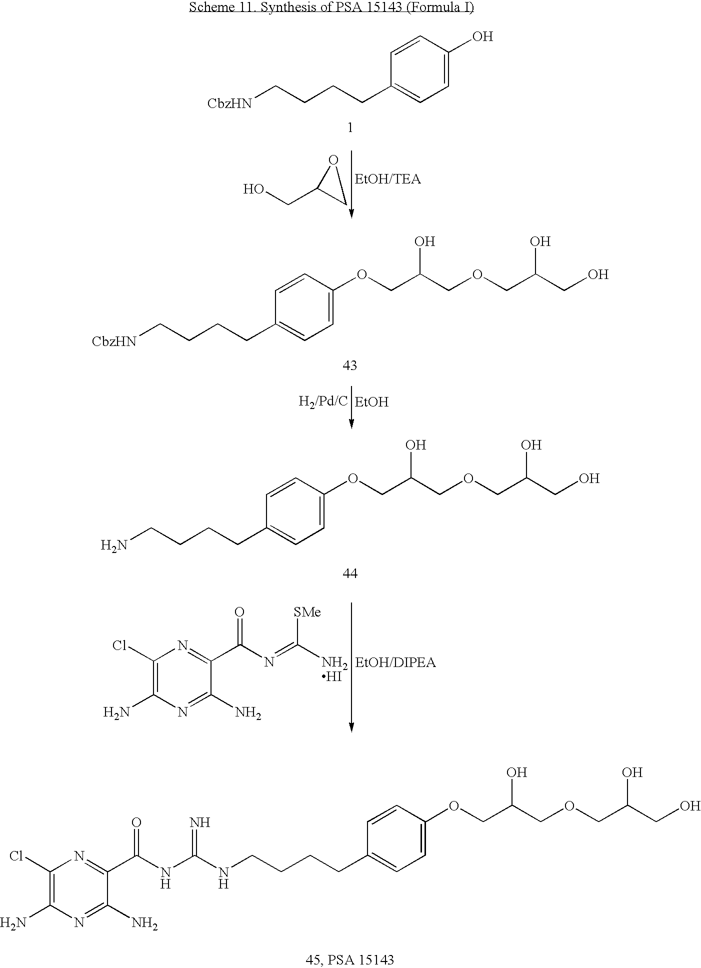

### Example 1

### Synthesis of N-(3,5-diamino-6-chloropyrazine-2-carbonyl)-N′-(4-{4-[3-(1H-tetrazol-5-yl)propoxy]phenyl}butyl)guanidine hydrochloride (PSA 17926)

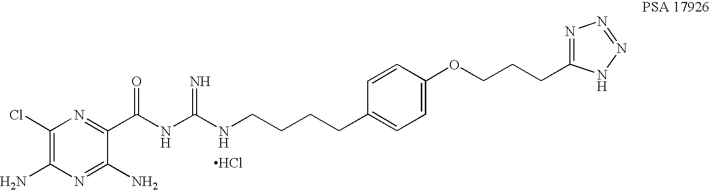

### {4-[4-(3-Cyanopropoxy)phenyl]butyl}carbamic acid benzyl ester (2)

A mixture of [4-(4-hydroxyphenyl)butyl]carbamic acid benzyl ester 1 (2.00 g, 6.70 mmol), 4-bromobutyronitrile (0.70 mL, 6.70 mmol), and potassium carbonate (1.00 g, 7.4 mmol) in DMF (10 mL), was stirred at 65° C. for 16 h. Solvent was removed by rotary evaporation and the residue was taken up in ethyl acetate, washed with water and brine, and concentrated under vacuum. The crude product was purified by flash silica gel column chromatography eluting with ethyl acetate/CH₂Cl₂ (1:9, v/v) to give the desired product 2 as a white solid (1.80 g, 75% yield). ¹H NMR (300 MHz, CDCl₃) δ 1.56 (m, 4H), 2.15 (m, 2H), 2.55 (m, 4H), 3.15 (m, 2H), 4.00 (m, 2H), 4.70 (br s, 1H), 5.10 (s, 2H), 6.80 (d, 2H), 7.05 (d, 2H), 7.30 (m, 5H). m/z (ESI): 367 [C₂₂H₂₆N₂O₃+H]⁺.

### (4-{4-[3-(1H-Tetrazol-5-yl)propoxy]phenyl}butyl)carbamic acid benzyl ester (3)

A mixture of {4-[4-(3-cyanopropoxy)phenyl]butyl}carbamic acid benzyl ester 2 (0.90 g, 2.5 mmol), sodium azide (0.50 g, 7.5 mmol), and ammonium chloride (0.40 g, 7.5 mmol) in DMF (7 mL), was stirred at 120° C. for 16 h. Inorganics were removed by vacuum filtration. The filtrate was diluted with ethyl acetate, and washed with water and brine. The organic solution was dried over Na₂SO₄, filtered and concentrated. The residue was taken up in ethyl acetate (5 mL) and diluted with hexanes (10 mL). Solid precipitates were collected by suction filtration and purified by flash silica gel column chromatography eluting with methanol/dichloromethane (1:50, v/v) to give the desired product 3 as a white solid (0.78 g, 76% yield). ¹H NMR (300 MHz, CD₃OD) δ 1.51 (m, 4H), 2.20 (m, 2H), 2.50 (m, 2H), 3.10 (m, 4H), 4.00 (m, 2H), 5.00 (s, 2H), 6.75 (d, 2H), 7.05 (d, 2H), 7.30 (m, 5H). m/z (ESI): 410 [C₂₂H₂₇N₅O₃+H]⁺.

### 4-{4-[3-(1H-Tetrazol-5-yl)propoxy]phenyl}butylamine (4)

A solution of (4-{4-[3-(1H-tetrazol-5-yl)propoxy]phenyl}butyl)carbamic acid benzyl ester 3 (0.30 g, 0.73 mmol) in methanol (20 ML) and dichloromethane (5 mL) was stirred at room temperature overnight under hydrogen atmosphere in the presence of 10% palladium-on-carbon catalyst (0.1 g, 50% wet). The catalyst was removed by suction filtration, and the filtrate was concentrated in vacuo to give the desired product 4 as a white solid (200 mg, 99% yield) which was used for the next step without further purification. m/z (ESI): 276 [C₁₄H₂₁N₅O+H]⁺.

### N-(3,5-Diamino-6-chloropyrazine-2-carbonyl)-N′-(4-{4-[3-(1H-tetrazol-5-yl)-propoxy]phenyl}butyl)guanidine hydrochloride (5, PSA 17926)

A solution of 4-{4-[3-(1H-tetrazol-5-yl)propoxy]phenyl}butylamine 4 (100 mg, 0.36 mmol) and triethylamine (0.15 mL, 0.39 mmol) in absolute ethanol (2 mL) was stirred at 60° C. for 5 min, after which 1-(3,5-diamino-6-chloropyrazine-2-carbonyl)-2-methyl-isothiourea hydriodide (150 mg, 0.39 mmol) was added in one portion. The reaction mixture was stirred at that temperature for 4 h and then cooled to room temperature. The reaction mixture was concentrated by rotary evaporation. The crude residue was washed with water and filtered. The filter cake was further washed with dichloromethane. A dark yellow solid (140 mg, 80% yield) thus obtained was slurried in a mixture of methanol and dichloromethane (5/95, v/v). The solid was collected by suction filtration, and 40 mg of such solid was mixed with 3% aqueous HCl (4 mL). The mixture was sonicated, stirred at room temperature for 15 min and filtered. The filter cake was dried under high vacuum to give N-(3,5-diamino-6-chloropyrazine-2-carbonyl)-N′-(4-{4-[3-(1H-tetrazol-5-yl)propoxy]phenyl}butyl)guanidine hydrochloride (5, PSA 17926) as a yellow solid. mp 125-127° C. (decomposed). ¹H NMR (300 MHz, CD₃OD) δ 1.70 (m, 4H), 2.22 (m, 2H), 2.60 (m, 2H), 3.10 (m, 2H), 4.00 (m, 2H), 6.70 (d, 2H), 7.09 (d, 2H). m/z (ESI): 488 [C₂₀H₂₆ClN₁₁O₂+H]⁺.

### Example 2

### Synthesis of dimethylthiocarbamic acid O-(4-{4-[N′-(3,5-diamino-6-chloropyrazine-2-carbonyl)guanidino]butyl}phenyl) ester (PSA 17846)

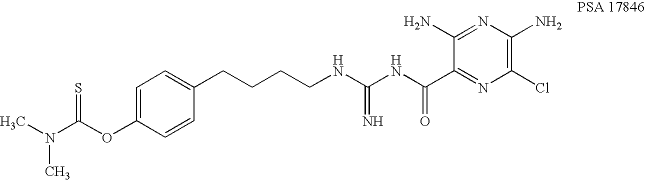

### 2-[4-(4-Hydroxyphenyl)butyl]isoindole-1,3-dione (8)

A mixture of 4-(4-aminobutyl)phenol hydrobromide 6 (8.2 g, 33.5mmol), phthalic anhydride 7 (5.0 g, 33.8 mmol), and triethylamine (4.6 mL, 33.5 mmol) in chloroform (50 mL) was stirred at reflux for 18 h, cooled to room temperature and concentrated by rotary evaporation. The residue was dissolved in acetic acid (50 mL) and stirred at 100° C. for 3 h. Solvent was evaporated and the resulting residue was purified by flash silica gel column chromatography eluting with CH₂Cl₂/EtOAc/hexanes (8:1:1, v/v) to give the desired product 8 as a white powder (4.1 g, 41% yield). ¹H NMR (300 MHz, DMSO-d₆) 6 1.57 (m, 4H), 2.46 (m, 2H), 3.58 (m, 2H), 6.64 (d, 2H), 6.95 (d, 2H), 7.82 (m, 4H), 9.12 (s, 1H). m/z (ESI): 296 [C₁₈H₁₇NO₃+H]⁺.

### Dimethylthiocarbamic acid O-{4-[4-(1,3-dioxo-1,3-dihydroisoindol-2-yl)butyl]-phenyl} ester (9)

A suspension of sodium hydride (60% in mineral oil, 0.44 g, 0.11 mmol) in anhydrous DMF (10 mL) was cooled to 0° C. and added to a solution of 2-[4-(4-hydroxyphenyl)-butyl]isoindole-1,3-dione 8 (2.95 g, 10 mmol) in DMF (15 mL). The mixture was stirred at 0° C. for 30 min and then at room temperature for an additional one hour. A solution of dimethylthiocarbamic acid chloride (1.35 g, 11 mmol) in DMF (10 mL) was then added. The reaction mixture was stirred at room temperature first for 16 h and then at 50° C. for 1 h, cooled back to room temperature and quenched with methanol (10 mL). The mixture was concentrated under vacuum and the residue was purified by flash silica gel column chromatography eluting with CH₂Cl₂/hexanes/EtOAc (10:1:0.2, v/v) to give the desired product 9 as a yellowish solid (2.27 g, 59% yield). ¹H NMR (300 MHz, CDCl₃) δ 1.72 (m, 4H), 2.67 (m, 2H), 3.33 (s, 3H), 3.45 (s, 3H), 3.71 (m, 2H), 6.95 (d, 2H), 7.18 (d, 2H), 7.70 (m, 2H), 7.84 (m, 2H). m/z (ESI): 383 [C₂₁H₂₂N₂O₃S+H]⁺.

### Dimethylthiocarbamic acid O-[4-(4-aminobutyl)phenyl] ester (10)

A mixture of dimethylthiocarbamic acid O-{4-[4-(1,3-dioxo-1,3-dihydroisoindol-2-yl)-butyl]phenyl} ester 9 (0.30 g, 0.80 mmol) and methylamine (2M in methanol, 10 mL, 20 mmol) was stirred at room temperature overnight. Solvent was removed by rotary evaporation and the residue was purified by flash silica gel column chromatography (Biotage) eluting with chloroform/methanol/concentrated ammonium hydroxide (10:1:0.1, v/v) to give dimethylthiocarbamic acid O-[4-(4-aminobutyl)phenyl] ester (10) as a clear colorless oil (118 mg, 46% yield). ¹H NMR (300 MHz, CD₃OD) δ 1.70 (m, 4H), 2.70 (m, 4H), 3.34 (s, 3H), 3.46 (s, 3H), 6.96 (d, 2H), 7.20 (d, 2H). m/z (ESI): 253 [Cl₃H₂₀N₂OS+H]⁺.

### Dimethylthiocarbamic acid O-(4-{4-[N′-(3,5-diamino-6-chloropyrazine-2-carbonyl)-guanidino]butyl}phenyl) ester (11, PSA 17846)

A solution of dimethylthiocarbamic acid O-[4-(4-aminobutyl)phenyl] ester 10 (115 mg, 0.45 mmol), triethylamine (0.30 mL, 2.2 mmol), and 1-(3,5-diamino-6-chloropyrazine-2-carbonyl)-2-methylisothiourea hydriodide (175 mg, 0.45 mmol) in anhydrous THF (6 mL) was stirred at reflux for 3 h and then cooled to room temperature. The reaction mixture was concentrated by rotary evaporation. The crude residue was purified by flash silica gel column chromatography (Biotage) eluting with chloroform/methanol/concentrated ammonium hydroxide (15:1:0.1, v/v) to give the desired product 11 as a yellow solid (180 mg, 86% yield). mp 102-105° C. ¹H NMR (300 MHz, CD₃OD) δ 1.70 (m, 4H), 2.65 (m, 2H), 3.20 (m, 2H), 3.30 (s, 3H), 3.40 (s, 3H), 6.95 (d, 2H), 7.20 (d, 2H). m/z (ESI): 465 [Cl₉H₂₅ClN₈O₂S+H]⁺.

### Example 3

### Synthesis of (2S)-(4-{4-[N′-(3,5-diamino-6-chloropyrazine-2-carbonyl)guanidino]-butyl}benzenesulfonylamino)-3-methylbutyramide (PSA 19008)

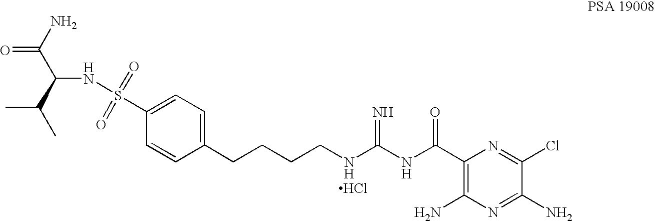

### 4-[4-(1,3-Dioxo-1,3-dihydroisoindol-2-yl)butyl]benzenesulfonyl chloride (13)

2-(4-Phenylbutyl)isoindole-1,3-dione 12 (1.9 g, 6.8 mmol) was added to chlorosulfonic acid (10 mL, 138 mmol) at 0° C. and the mixture was stirred for 1 h at the temperature. After storing in refrigerator at −5° C. overnight, the reaction mixture was poured onto crushed ice (100 g) and precipitates were collected by a suction filtration and dried under high vacuum to afford the desired product 13 (2.48 g, 99% yield). ¹H NMR (300 MHz, CDCl₃) δ 1.70 (m, 4H), 2.78 (m, 2H), 3.70 (m, 2H), 7.40 (d, 2H) 7.70 (d, 2H), 7.85 (d, 2H), 7.95 (d, 2H).

### (2S)-{4-[4-(1,3-Dioxo-1,3-dihydroisoindol-2-yl)butyl]benzenesulfonylamino}-3-methylbutyramide (14)

4-[4-(1,3-Dioxo-1,3-dihydroisoindol-2-yl)butyl]benzenesulfonyl chloride 13 (0.45 g, 1.19 mmol) was dissolved in dry DMF (5 mL), and added to a solution of N-methylmorpholine (3 mL) and (2S)-amino-3-methylbutyramide (0.18 g, 1.19 mmol) in DMF (10 mL). The reaction mixture was stirred at room temperature for 66 h. Solvent was removed by rotary evaporation and the residue was purified by flash silica gel chromatography eluting with chloroform/methanol/concentrated ammonium hydroxide (15:1:0.1, v/v) to give the desired product 14 as a white powder (0.41 g, 73% yield). ¹H NMR (300 MHz, DMSO-d₆) δ 0.72 (d, 3H), 0.76 (d, 3H), 1.77 (m, 4H), 1.79 (m, 1H), 2.68 (m, 2H), 3.40 (m, 1H), 3.60 (m, 2H), 6.92 (s, 1H), 7.21 (s, 1H), 7.34 (d, 2H) 7.50 (d, 1H), 7.65 (d, 2H), 7.82 (m, 4H).

### (2S)-[4-(4-Aminobutyl)benzenesulfonylamino]-3-methylbutyramide (15)

A mixture of (2S)-{4-[4-(1,3-dioxo-1,3-dihydroisoindol-2-yl)butyl]-benzenesulfonylamino}-3-methylbutyramide 14 (0.40 g, 0.87 mmol) and methylamine (2 M in methanol, 20 mL, 40 mmol) was stirred at room temperature overnight. Solvent was removed by rotary evaporation and the residue was purified by flash silica gel column chromatography eluting with chloroform/methanol/concentrated ammonium hydroxide (3:1:0.1, v/v) to give (2S)-[4-(4-aminobutyl)benzenesulfonylamino]-3-methyl-butyramide (15) as a white powder (156 mg, 54% yield). ¹H NMR (300 MHz, CD₃OD) δ 0.85 (d, 3H), 0.87 (d, 3H), 1.66 (m, 4H), 1.90 (m, 1H), 2.69 (m, 4H), 3.51 (d, 1H), 7.35 (d, 2H) 7.75 (d, 2H). m/z (ESI): 328 [Cl₅H₂₅N₃O₃S+H]⁺.

### (2S)-(4-{4-[NM-(3,5-Diamino-6-chloropyrazine-2-carbonyl)guanidino]butyl}-benzenesulfonylamino)-3-methylbutyramide (16, PSA 19008)

A solution of (2S)-[4-(4-aminobutyl)benzenesulfonylamino]-3-methylbutyramide 15 (156 mg, 0.47 mmol), diisopropylethylamine (0.60 mL, 3.0 mmol), and 1-(3,5-diamino-6-chloropyrazine-2-carbonyl)-2-methylisothiourea hydriodide (230 mg, 0.61 mmol) in absolute ethanol (8 mL) was stirred at 70° C. for 5 h and then cooled to room temperature. The reaction mixture was concentrated by rotary evaporation. The crude residue was washed with water, filtered and the crude solid product was purified by flash silica gel column chromatography eluting with chloroform/methanol/concentrated ammonium hydroxide (5:1:0.1, v/v) to give the desired product as a yellow solid (137 mg, 54% yield). Part of the solid (86 mg) was further purified by semi-preparative HPLC (acetonitrile/water/0.1% TFA) to give the analytical pure sample which was then co-evaporated with 5% aqueous HCl to give the hydrochloride salt 16. mp 154-156° C. (decomposed). ¹H NMR (300 MHz, CD₃OD) δ 0.85 (d, 3H), 0.86 (d, 3H), 1.70 (m, 4H), 1.90 (m, 1H), 2.75 (m, 2H), 3.32 (m, 2H), 3.52 (d, 1H), 7.35 (d, 2H), 7.75 (d, 2H). m/z (ESI): 540 [C₂₁H₃₀ClN₉O₄S+H]⁺. [α]_{D}²⁵+5.20 (c 0.50, MeOH).

### Example 4

### Synthesis of 2-(4-{4-[N′-(3,5 -diamino-6-chloropyrazine-2-carbonyl)guanidino]-butyl}phenoxy)-N-phenylacetamide (PSA 17482)

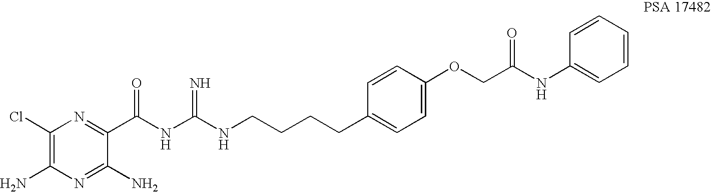

### 4-(4-Phenylcarbamoylmethoxyphenyl)butyl]carbamic acid benzyl ester (18)

A mixture of [4-(4-benzyloxycarbonylaminobutyl)phenoxy] acetic acid (300 mg, 0.84 mmol), aniline (0.15 mL, 1.70 mmol), DMAP (60 mg, 0.50 mmol) and EDC.HCl (320 mg, 1.70 mmol) in CH₂Cl₂ (30 mL) was stirred at room temperature for 66 h. The reaction mixture was concentrated under vacuum and the residue was subjected to flash silica gel column chromatography eluting with methanol/CH₂Cl₂ (1:99, v/v) to give the desired amide 18 as a white solid (360 mg, 99% yield). ¹H NMR (300 MHz, CDCl₃) δ 1.55 (m, 4H), 2.60 (m, 2H), 3.20 (m, 2H), 4.58 (s, 2H), 4.70 (br s, 1H), 5.10 (s, 2H), 6.88 (d, 2H), 7.15 (m, 3H), 7.35 (m, 7H), 7.58 (s, 2H), 8.25 (s, 1H). m/z (ESD): 433 [C₂₆H₂₈N₂O₄+H]⁺.

### 2-[4-(4-Aminobutyl)phenoxy]-N-phenylacetamide (19)

A solution of [4-(4-phenylcarbamoylmethoxyphenyl)butyl]carbamic acid benzyl ester 18 (0.30 g, 0.69 mmol) in ethanol (10 mL), THF (6 mL), and acetic acid (2 mL) was stirred at room temperature for 2 h under hydrogen atmosphere in the presence of 10% Pd/C catalyst (0.2 g, 50% wet). The catalyst was removed by suction filtration and the filtrate was concentrated in vacuo. The residue was purified by flash silica gel column chromatography eluting with CH₂Cl₂/methanol/concentrated ammonium hydroxide (30:1:0, 30:1:0.3, v/v) to give the desired amine 19 as a white solid (200 mg, 97% yield). ¹H NMR (300 MHz, CD₃OD) δ 1.60 (m, 4H), 2.55 (m, 2H), 2.70 (m, 2H), 4.60 (s, 2H), 6.88 (d, 2H), 7.15 (m, 3H), 7.35 (m, 2H), 7.58 (d, 2H), 8.25 (s, 1H). m/z (EST): 299 [Cl₈H₂₂N₂O₂+H]⁺.

### 2-(4-{4-[NA-(3,5-Diamino-6-chloropyrazine-2-carbonyl)guanidino]butyl}phenoxy)-N-phenylacetamide (20, PSA 17482)

A solution of 2-[4-(4-aminobutyl)phenoxy]-N-phenylacetamide 19 (100 mg, 0.35 mmol) and triethylamine (0.14 mL, 1.00 mmol) in absolute ethanol (2 mL) was stirred at 60° C. for 30 min, after which 1-(3,5-diamino-6-chloropyrazine-2-carbonyl)-2-methyl-isothiourea hydriodide (140 mg, 0.37 mmol) was added in one portion. The reaction mixture was stirred at that temperature for 4 h, cooled to room temperature, and concentrated by rotary evaporation. The crude residue was triturated with water and filtered. The filter cake was purified by flash silica gel column chromatography eluting with dichloromethane/methanol/concentrated ammonium hydroxide (500:10:0, 500:10:1, 200:10:1, v/v) to give 2-(4-{4-[N-(3,5-diamino-6-chloro-pyrazine-2-carbonyl)guanidino]butyl}phenoxy)-N-phenylacetamide (20, PSA 17482) as a yellow solid (120 mg, 67% yield). mp 168-170° C. ¹H NMR (300 MHz, DMSO-d₆) δ 1.55 (m, 4H), 2.55 (m, 2H), 3.16 (m, 2H), 4.65 (s, 2H), 6.60 (br s, 2H), 6.90 (d, 2H), 7.08 (m, 2H), 7.15 (d, 2H), 7.30 (m, 5H), 7.60 (d, 2H), 9.00 (br s, 1H), 10.00 (br s, 1H). m/z (ESI): 511 [C₂₄H₂₇ClN₈O₃+H]⁺.

### Example 5

### Synthesis of N-(3,5-diamino-6-chloropyrazine-2-carbonyl)-N′-(4-{4-[3-(1H-imidazol-2-yl)propoxy]phenyl}butyl)guanidine (PSA 23022)

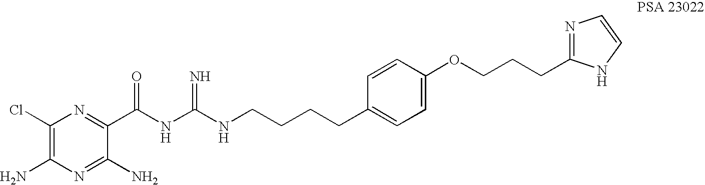

### 4-{4-[3-(1H-Imidazol-2-yl)propoxylphenyl}butylamine (21)

Compound 2 (0.156 g, 0.425 mmol) was dissolved in anhydrous ethanol (10 mL). To the solution was bubbled anhydrous HCl gas for 3 min. The reaction vessel was sealed and the mixture was stirred at room temperature for 48 h, and then concentrated to dryness under vacuum. The resulting residue was dissolved in anhydrous methanol (5 mL). To the newly formed solution was added 2,2-dimethoxyethylamine (0.097 mL, 0.891 mmol) in one portion. After stirring at room temperature overnight, temperature was raised to reflux which was maintained for another 3 h before the mixture was cooled to ambient temperature. Solvent was removed under vacuum and the residue was treated with 1.2 N HCl aqueous solution at 80° C. for 2 hours. The mixture was then cooled to ambient temperature again and neutralized to pH 9 with powder K₂CO₃. Water was completely removed under vacuum and the residue was dissolved in methanol. The methanol solution was loaded onto silica gel, and the product was eluted with a mixture of concentrated ammonium hydroxide/MeOH/CH₂Cl₂ (1.8:18:81.2, v/v), affording the product 21 (27 mg, 23% overall yield) as an off-white solid. ¹H NMR (300 MHz, CD₃OD): δ 1.60 (m, 4H), 2.14 (m, 2H), 2.56 (t, 2H), 2.76 (t, 2H), 2.86 (t, 2H), 3.94 (t, 2H), 6.79 (d, 2H), 6.91 (s, 2H), 7.08 (d, 2H). m/z (APCI): 274 [Cl₆H₂₃N₃O+H]⁺.

### N-(3,5-Diamino-6-chloropyrazine-2-carbonyl)-N′-(4-(4-[3-(1H-imidazol-2-yl)propoxylphenyl}butyl)guanidine (22, PSA 23022)

Compound 21 (23 mg, 0.084 mmol) was dissolved in a mixture of ethanol (3 mL) and Hunig's base (0.074 mL, 0.421 mmol) at 65° C. over 15 min. To the solution was added 1-(3,5-diamino-6-chloropyrazine-2-carbonyl)-2-methlisothiourea hydriodide (43 mg, 0.109 mmol) and the resulting mixture was stirred at the above temperature for an additional 3 h before all liquid was removed under vacuum. The residue was chromatographed on silica gel, eluting with a mixture of concentrated ammonium hydroxide/methanol/dichloromethane (1.5:15:63.5, v/v), to afford the desired product 22 (34 mg, 83% yield) as a yellow solid. mp 123-126° C. (decomposed), ¹H NMR (300 MHz, CD₃OD): δ 1.62 (m, 4H), 2.14 (m, 2H), 2.58 (t, 2H), 2.88 (t, 2H), 3.21(t, 2H), 3.94(t, 2H), 6.77 (d, 2H), 6.90 (s, 2H), 7.06 (d, 2H). m/z (APCI): 486 [C₂₂H₂₈ClN₉O₂+H]⁺.

### Example 6

### Synthesis of 2-(4-{4-[N-(3,5-diamino-6-chloropyrazine-2-carbonyl)guanidino]-butyl}phenoxy)-N,N-bis-(2-hydroxyethyl)acetamide (PSA 16826)

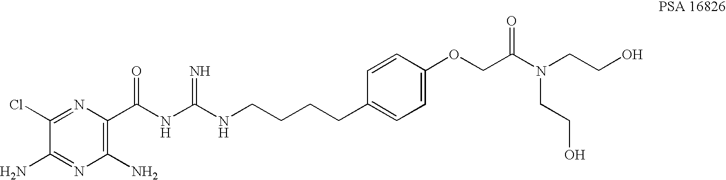

[4-(4-{[N,N-Bis-(2-hydroxyethyl)carbamoyl]methoxy}phenyl)butyl]carbamic acid benzyl ester (25)

A solution of [4-(4-benzyloxycarbonylaminobutyl)phenoxy]acetic acid ethyl ester 23 (0.3 g, 0.78 mmol), 2-(2-hydroxyethylamino)ethanol 24 (0.15 mL, 1.6 mmol), and ethanol (20 mL) was heated at 70° C. for 72 hours. Solvent was evaporated in vacuo. The residue was purified by flash chromatography (silica gel, dichloromethane/methanol, 100:5, v/v) to provide [4-(4-{[N,N-bis-(2-hydroxyethyl)carbamoyl]methoxy}phenyl)-butyl]carbamic acid benzyl ester 25 [0.19 g, 100% based on the recovered starting material (0.13 g)] as a pale yellow solid. ¹H NMR (300 MHz, CD₃OD) δ 1.65 (m, 4H), 2.50 (m, 2H), 3.20 (m, 2H), 3.55 (m, 4H), 3.75 (m, 4H), 4.80 (s, 2H), 5.10 (s, 2H), 6.85 (d, 2H), 7.10 (d, 2H), 7.40 (m, 5H). m/z (ESI): 445 [C₂₄H₃₂N₂O₆+H]⁺.

### 2-[4-(4-Aminobutyl)phenoxy]-N,N-bis-(2-hydroxyethyl)acetamide (26)

To a degassed solution of [4-(4-{[N,N-bis-(2-hydroxyethyl)carbamoyl]methoxy}phenyl)-butyl]carbamic acid benzyl ester 25 (0.19 g, 0.43 mmol) in ethanol (4 mL) was added 10% palladium on activated carbon (0.1 g, 50% wet). The mixture was hydrogenated overnight at atmospheric hydrogen. The catalyst was filtered through a pad of diatomaceous earth and the solvent was evaporated in vacuo. The residue was purified by flash chromatography (silica gel, 20-5:1:0.1-1 dichloromethane/methanol/concentrated ammonium hydroxide, v/v) to provide 26 (0.09 g, 72%) as a colorless oil. ¹H NMR (300 MHz, CD₃OD) δ 1.56 (m, 4H), 2.56 (t, 2H), 2.65 (t, 1H), 3.29 (m, 1H), 3.55 (m, 4H), 3.72 (m, 4H), 4.90 (s, 2H), 6.86 (d, 2H), 7.09 (d, 2H). m/z (ESI): 311 [C₁₆H₂₆N₂O₄+H]⁺.

### 2-(4-{4-[N′-(3,5-Diamino-6-chloropyrazine-2-carbonyl)guanidino]butyl}phenoxy)-N,N-bis-(2-hydroxyethyl)acetamide (27, PSA 16826)

1-(3,5-Diamino-6-chloropyrazine-2-carbonyl)-2-methylisothiourea hydriodide (0.13 g, 0.33 mmol) was added to a solution of 2-[4-(4-aminobutyl)phenoxy]-N,N-bis-(2-hydroxyethyl)acetamide 26 (0.09 g, 0.3 mmol), triethylamine (0.12 mL), and ethanol (1.7 mL). The reaction mixture was stirred at 60° C. for 3 h. The solvent was evaporated in vacuo. The residue was triturated with water and then purified by flash chromatography (silica gel, 20-10:1:0-0.2 CH₂Cl₂/methanol/concentrated ammonium hydroxide, v/v) to provide 2-(4-{4-[N′-(3,5-diamino-6-chloropyrazine-2-carbonyl)guanidino]butyl}-phenoxy)-N,N-bis-(2-hydroxyethyl)acetamide 27 (0.1 g, 64%) as a yellow solid. mp 1 14-116° C. ¹H NMR (300 MHz, CD₃OD) δ 1.70 (m, 4H), 2.60 (m, 2H), 3.32 (m, 2H), 3.50 (m, 4H), 3.70 (m, 4H), 4.81 (s, 2H), 6.85 (d, 2H), 7.10 (d, 2H). m/z (ESD): 523 [C₂₂H₃₁ClN₈O₅+H]⁺.

### Example 7

### Synthesis of 2-(4-{4-[N-(3,5-diamino-6-chloropyrazine-2-carbonyl)guanidino]-butyl}phenoxy)-N,N-dimethylacetamide hydrochloride (PSA 16313)

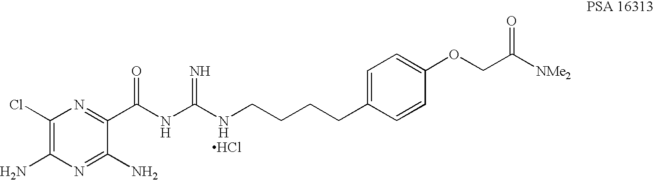

### [4-(4-Dimethylcarbamoylmethoxyphenyl)butyl]carbamic acid benzyl ester (28)

A mixture of [4-(4-benzyloxycarbonylaminobutyl)phenoxy]acetic acid ethyl ester 23 (0.50 g, 1.3 mmol) and dimethylamine (2.0 M in THF, 10 mL, 20 mmol) in a sealed tube was heated at 55° C. for 48 h. The solvent was evaporated in vacuo. The residue was purified by flash chromatography (silica gel, ethyl acetate/CH₂Cl₂, 1:4, 1:3, v/v) to provide [4-(4-dimethylcarbamoylmethoxyphenyl)butyl]carbamic acid benzyl ester 28 (0.26 g, 52% yield) as a white solid. ¹H NMR (300 MHz, CDCl₃) δ 1.55 (m, 4H), 2.55 (m, 2H), 2.90 (s, 3H), 3.05 (s, 3H), 3.20 (m, 2H), 4.65 (s, 2H), 5.08 (s, 2H), 6.80 (d, 2H), 7.05 (d, 2H), 7.35 (m, 5H).

### 2-[4-(4-Aminobutyl)phenoxy]-N,N-dimethylacetamide (29)

To a degassed solution of [4-(4-dimethylcarbamoylmethoxyphenyl)butyl]carbamic acid benzyl ester (28) (0.26 g, 0.68 mmol) in ethanol (10 mL) was added 10% palladium on activated carbon (0.1 g, 50% wet). The mixture was stirred at room temperature overnight under atmospheric hydrogen. The catalyst was filtered through a pad of diatomaceous earth and the solvent was evaporated in vacuo. The residue was purified by flash chromatography (silica gel, dichloromethane/methanol/concentrated ammonium hydroxide, 100:5:1, v/v) to provide 2-[4-(4-aminobutyl)phenoxy]-N,N-dimethylacetamide 29 (100 mg, 60% yield) as a white solid. ¹H NMR (300 MHz, CD₃OD) δ 1.55 (m, 4H), 2.55 (m, 2H), 2.66 (m, 2H), 2.90 (s, 3H), 3.05 (s, 3H), 4.70 (s, 2H), 6.80 (d, 2H), 7.05 (d, 2H). m/z (ESI): 251 [Cl₄H₂₂N₂O₂+H]⁺.

### 2-(4-{4-[N′-(3,5-Diamino-6-chloropyrazine-2-carbonyl)guanidino]butyl}phenoxy)-N,N-dimethylacetamide hydrochloride (30, PSA 16313)

A solution of 2-[4-(4-aminobutyl)phenoxy]-N,N-dimethylacetamide 29 (67 mg, 0.27 mmol) in absolute ethanol (1 mL) was stirred at 65° C. for 30 min, after which 1-(3,5-diamino-6-chloropyrazine-2-carbonyl)-2-methylisothiourea hydriodide (110 mg, 0.29 mmol) was added in one portion. The reaction mixture was stirred at that temperature for 3 h and then cooled to room temperature. The reaction mixture was concentrated by rotary evaporation. The crude residue was triturated with water and filtered. The filter cake was purified by flash silica gel column chromatography eluting with dichloromethane/methanol/concentrated ammonium hydroxide (200:10:0, 200:10:1, v/v) to give 2-(4-{4-[N′-(3,5-diamino-6-chloropyrazine-2-carbonyl)guanidino]butyl}-phenoxy)-N,N-dimethylacetamide as a yellow solid (35 mg, 28% yield). This solid was dissolved in methanol (2 mL) and added to 4 N aqueous HCl (4 drops). Concentration in vacuo gave 2-(4-{4-[NA-(3,5-diamino-6-chloropyrazine-2-carbonyl)-guanidino]butyl}phenoxy)-N,N-dimethylacetamide hydrochloride (30, PSA 16313). mp 130-132° C. (decomposed). ¹H NMR (300 MHz, CD₃OD) δ 1.69 (m, 4H), 2.60 (m, 2H), 2.95 (s, 3H), 3.10 (s, 3H), 3.35 (m, 2H), 4.75 (s, 2H), 6.80 (d, 2H), 7.10 (d, 2H). m/z (ESI): 463 [C₂₀H₂₇ClN₈O₃+H]⁺.

### Example 8

### Synthesis of 2-(4-{4-[N′-(3,5-diamino-6-chloropyrazine-2-carbonyl)guanidino]-butyl}phenoxy)-N-(1H-imidazol-2-yl)acetamide dihydrochloride (PSA 16437)

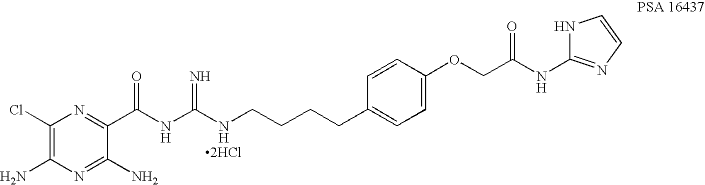

### [4-(4-tert-Butoxycarbonylaminobutyl)phenoxy]acetic acid methyl ester (32)

A mixture of [4-(4-hydroxyphenyl)butyl]carbamic acid tert-butyl ester 31 (1.00 g, 3.78 mmol), potassium carbonate (0.627 g, 4.54 mmol), sodium iodide (0.567 g, 3.78 mmol), and methyl bromoacetate (0.40 mL, 4.21 mmol) in anhydrous DMF (8 mL) was stirred at room temperature for 14 h. The reaction mixture was then diluted with ethyl acetate (100 mL) and hexanes (20 mL), washed with water (20 mL×4) and brine (30 mL), and concentrated under reduced pressure to afford the desired product 32 as a yellow oil (1.28 g, 100% yield) which was used for the next step without further purification. ¹H NMR (300 MHz, CDCl₃) δ 1.40 (s, 9H), 1.41-1.65 (m, 4H), 2.49-2.60 (m, 2H), 3.02-3.16 (m, 2H), 3.79 (s, 3H), 4.45 (br s, 1H), 4.59 (s, 2H), 6.79 (d, 2H), 7.05 (d, 2H). m/z (ESI): 338 [C₁₈H₂₇NO₅+H]⁺.

### [4-(4-tert-Butoxycarbonylaminobutyl)phenoxy]acetic acid (33)

A solution of [4-(4-tert-butoxycarbonylaminobutyl)phenoxy]acetic acid methyl ester 32 (1.28 g, 3.78 mmol) in methanol (80 mL) was added with crushed potassium hydroxide (2.50 g, 85%, 37.8 mmol) and the mixture was stirred at room temperature for 5 h. Solvent was removed by rotary evaporation. The residue was taken up in water and acidified to pH ˜1 with 6N aqueous HCl, and extracted with dichloromethane. The combined organics were washed with brine, dried over Na₂SO₄, and concentrated to complete dryness to afford the desired product 33 as a white solid (1.19 g, 97% yield). ¹H NMR (300 MHz, CD₃OD) δ 1.41 (s, 9H), 1.42-1.70 (m, 4H), 2.45-2.60 (m, 2H), 3.00-3.20 (m, 2H), 4.60 (s, 2H), 6.80 (d, 2H), 7.08 (d, 2H). m/z (ESI): 322 [C₁₇H₂₅NO₅−H]⁻.

### (4-{4-[(1H-Imidazol-2-yl-carbamoyl)methoxy]phenyl}butyl)carbamic acid tert-butyl ester (34)

[4-(4-tert-Butoxycarbonylaminobutyl)phenoxy]acetic acid 33 (1.19 g, 3.68 mmol) was dissolved in anhydrous THF (10 mL), CH₂Cl₂ (10 mL) and CH₃CN (5 mL). To the solution were sequentially added HOAt (200 mg, 1.47 mmol), DMAP (135 mg, 1.10 mmol), and diisopropylethylamine (3.2 mL, 18.40 mmol), followed by the addition of EDC.HCl (1.03 g, 5.35 mmol). The reaction mixture was stirred at room temperature for 15 min. Amino imidazole sulfate (583 mg, 4.41 mmol) was then added and stirring was continued for 48 h. Solvents were removed by rotary evaporation. The residue was taken up in CH₂Cl₂ (250 mL), washed with water and brine, and concentrated under reduced pressure. Flash silica gel column chromatography eluting with methanol/dichloromethane (1:30, 1:20, v/v) gave the desired amide as a white solid (0.95 g, 66% yield). ¹H NMR (300 MHz, CD₃OD) δ 1.40 (s, 9H), 1.42-1.70 (m, 4H), 2.48-2.60 (m, 2H), 3.00-3.20 (m, 2H), 4.65 (s, 2H), 6.79-6.89 (m, 4H), 7.10 (d, 2H). m/z (ESI): 389 [C₂₀H₂₈N₄O₄+H]⁺.

### 2-[4-(4-Aminobutyl)phenoxy]-N-(1H-imidazol-2-yl)acetamide dihydrochloride (35)

(4-{4-[(1H-Imidazol-2-yl-carbamoyl)methoxy]phenyl}butyl)carbamic acid tert-butyl ester 34 (950 mg, 2.45 mmol) was treated with HCl (4 M in dioxane, 24 mL, 96 mmol) at room temperature for 12 h. The reaction mixture was concentrated in vacuo and further co-evaporated with dichloromethane and methanol, and dried under high vacuum. The desired product was obtained as a white solid (779 mg, 98%) and used directly without flrther purification. ¹H NMR (300 MHz, CD₃OD) δ 1.59-1.74 (m, 4H), 2.55-2.67 (m, 2H), 2.85-2.98 (m, 2H), 4.80 (s, 2H), 7.00 (d, 2H), 7.18 (d, 2H), 7.19 (s, 2H). m/z (ESI): 289 [C₁₅H₂₀N₄O₂+H]⁺.

### 2-(4-{4-[N′-(3,5-Diamino-6-chloropyrazine-2-carbonyl)guanidino]butyl}phenoxy)-N-(1H-imidazol-2-yl)acetamide dihydrochloride (36, PSA 16437)

A solution of 2-[4-(4-aminobutyl)phenoxy]-N-(1H-imidazol-2-yl)acetamide dihydrochloride 35 (99 mg, 0.27 mmol) and diisopropylethylamine (0.27 mL, 1.53 mmol) in absolute ethanol (4 mL) and anhydrous methanol (3 mL) was stirred at 70° C. for 30 min, after which 1-(3,5-diamino-6-chloropyrazine-2-carbonyl)-2-methylisothiourea hydriodide (130 mg, 0.34 mmol) was added in one portion. The reaction mixture was stirred for 3 h and then cooled to room temperature. The yellow insolubles were removed by suction filtration and the liquid filtrate was concentrated by rotary evaporation. The crude residue was purified by flash silica gel column chromatography eluting with dichloromethane/methanol/concentrated ammonium hydroxide (200:10:0, 200:10:1, 150:10:1, and 100:10:1, v/v) to give 2-(4-{4-[N′-(3,5-diamino-6-chloropyrazine-2-carbonyl)guanidino]butyl}phenoxy)-N-(1H-imidazol-2-yl)-acetamide as a yellow solid (44 mg, 29% yield). The free base thus obtained was dissolved in methanol and treated with 4 drops of 4 N aqueous HCl. The solution was concentrated under reduced pressure and further dried under vacuum to give the final compound 36. mp 172-174° C. ¹H NMR (300 MHz, CD₃OD) δ 1.61-1.77 (m, 4H), 2.58-2.70 (m, 2H), 3.32-3.40 (m, 2H), 4.80 (s, 2H), 7.00 (d, 2H), 7.18 (d, 2H), 7.20 (s, 2H). m/z (ESI): 501 [C₂₁H₂₅ClN₁₀O₃+H]⁺.

### Example 9

### Synthesis of N-carbamoylmethyl-2-(4-{4-[N′-(3,5-diamino-6-chloropyrazine-2-carbonyl)guanidino]butyl}phenoxy)acetamide (PSA 16314)

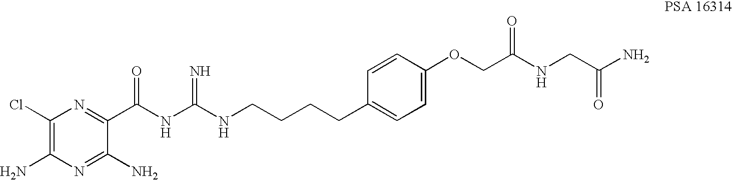

### (4-{4-[(Carbamoylmethylcarbamoyl)methoxy]phenyl}butyl)carbamic acid benzyl ester (37)

Compound 1 (0.50 g, 1.77 mmol) was dissolved in DMF (10 mL). To the solution was added crushed NaOH (0.107 g, 2.66 mmol). The mixture was stirred at room temperature for 30 min. 2-Bromoacetamide (0.367 g, 2.66 mmol) was added. The reaction was further stirred at room temperature overnight, quenched with water (2 mL) and partitioned between water and dichloromethane (each 50 mL). The organic layer was separated, washed with water (2×50 mL), dried over anhydrous Na₂SO₄ and concentrated under vacuum. The residue was purified on silica gel, eluting with a mixture of methanol/dichloromethane (7:93, v/v), to afford the desired product 37 (0.131 g, 18% yield) as a white solids. ¹H NMR (300 MHz, CDCl₃): δ 1.58 (m, 4H), 2.60 (t, 2H), 3.20 (m, 2H), 4.04 (d, 2H), 4.54 (s, 2H), 4.75 (br, 2H), 5.12 (s, 2H), 5.43 (br, 1H), 5.80 (br, 1H), 6.85 (d, 2H), 7.12 (d, 2H), 7.36 (m, 5H). m/z (APCI): 414 [C₂₂H₂₇N₃O₅+H]⁺.

### 2-[4-(4-Aminobutyl)phenoxy]-N-carbamoylmethylacetamide (38)

Compound 37 (130 mg, 0.314 mmol) was dissolved in EtOH and THF (14 mL, 1/1 ratio). The reaction vessel was purged with nitrogen both before and after the catalyst (100 mg, 10% Pd/C, 50% wet) was added. The mixture was stirred under hydrogen atmosphere (1 atm) overnight. After purging with nitrogen, the catalyst was vacuum filtered and washed with ethanol (3×5 mL). The combined filtrates were concentrated under vacuum. The residue was chromatographed on silica gel, eluting with a mixture of concentrated ammonium hydroxide/methanol/dichloromethane (2:20:88, v/v), to afford the desired product 38 (80 mg, 91% yield) as a white solid. ¹H NMR (300 MHz, CD₃OD): δ 1.62 (m, 4H), 2.60 (t, 2H), 2.75 (t, 2H), 3.92 (s, 2H), 4.54 (s, 2H), 6.92 (d, 2H), 7.14 (d, 2H).

### N-Carbamoylmethyl-2-(4-{4-[N′-(3,5-diamino-6-chloropyrazine-2-carbonyl)-guanidino]butyl}phenoxy)acetamide (39, PSA 16314)

Compound 38 (79 mg, 0.283 mmol) was dissolved in a mixture of absolute ethanol (5 mL) and Hunig's base (0.25 mL, 1.41 mmol) at 65° C. over 10 min. To the solution was added 1-(3,5-diamino-6-chloropyrazine-2-carbonyl)-2-methylisothiourea hydriodide (132 mg, 0.34 mmol) in one portion. The newly resulting reaction mixture was continuously stirred for an additional 2 h before it was cooled down to ambient temperature and subsequently concentrated under vacuum. The resulting residue was purified by chromatography eluting with methanol/dichloromethane/concentrated ammonium hydroxide (10/2/88, v/v) to afford the free base (93 mg, 67% yield) as a yellow solid. The HCl salt was made using the following procedure: 45 mg of the free base was suspended in ethanol (2 mL) and treated with concentrated HCl (12 N, 0.5 mL) for 10 min. All liquid was then completely removed under vacuum to afford 39 (47 mg). mp 178-180° C. (decomposed). ¹H NMR (300 MHz, DMSO-d₆): δ 1.61 (m, 4H), 2.58 (t, 2H), 3.32 (m, 2H), 3.70 (s, 2H), 4.48 (s, 2H), 6.93 (d, 2H), 7.08 (br, 1H), 7.13 (d, 2H), 7.36 (br, 1H), 7.44 (br, 2H), 8.17 (t, 1H), 8.74 (br, 1H), 8.90 (br, 2H), 9.18 (t, 1H), 10.48 (br, 1H). m/z (APCI): 492 [C₂₀H₂₆ClN₉O₄+H]⁺.

### Example 10

### Synthesis of N-[4-(4-cyanomethoxyphenyl)butyl]-N′-(3,5-diamino-6-chloro-pyrazine-2-carbonyl)guanidine (PSA 16208)

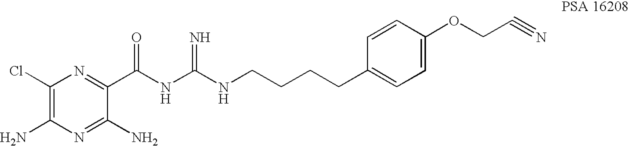

### [4-(4-Cyanomethoxyphenyl)butyl]carbamic acid tert-butyl ester (40)

A mixture of [4-(4-hydroxyphenyl)butyl]carbamic acid tert-butyl ester 31 (0.365 g, 1.37 mmol) and Cs₂CO₃ (0.672 g, 2.06 mmol) in anhydrous DMF (8 mL) was heated at 65° C. for 30 min. Iodoacetonitrile (0.276 g, 1.651 mmol) was then added to the mixture in one portion. The mixture was stirred at 65° C. overnight, and then cooled to room temperature. The precipitated solid was filtered, and the filtrate was partitioned between water and dichloromethane (each 50 mL). The organic layer was separated, washed with brine (3×50 mL), dried over anhydrous Na₂SO₄ and concentrated under vacuum. The residue was chromatographed on silica gel, eluting with a mixture of diethyl ether/dichloromethane (6:94, v/v), to afford the desired product 40 (0.109 g, 38% yield) as a colorless viscous oil. ¹H NMR (300 MHz, CDCl₃): δ 1.43 (s, 9H), 1.57 (m, 4H), 2.60 (t, 2H), 3.15 (m, 2H), 4.49 (br, 1H), 4.75 (s, 2H), 6.91 (d, 2H), 7.13 (d, 2H).

### [4-(4-Aminobutyl)phenoxy]acetonitrile (41)

Compound 40 (0.105 g, 0.345 mmol) was dissolved in dichloromethane (10 mL). Trifluoroacetic acid (2 mL) was added in one portion. The mixture was stirred at room temperature for 2 h, and then concentrated under vacuum to dryness. The crude residue was used directly without further purification. ¹H NMR (300 MHz, CD₃OD): δ 1.60-1.75 (m, 4H), 2.65 (t, 2H), 2.92 (t, 2H), 4.38 (s, 2H), 6.96 (d, 2H), 7.20 (d, 2H). m/z (APCI): 205 [C₁₂H₁₆N₂O+H]⁺.

### N-[4-(4-Cyanomethoxyphenyl)butyl]-N′-(3,5-diamino-6-chloropyrazine-2-carbonyl)guanidine (42, PSA 16208)

A mixture of compound 41 (0.070 g, 0.345 mmol) and Hunig's base (0.3 mL, 1.72 mmol) in anhydrous ethanol was heated at 65° C. for 20 min. To the solution was added 1-(3,5-diamino-6-chloropyrazine-2-carbonyl)-2-methylisothiourea hydriodide (0.148 g, 0.379 mmol) in one portion. The heating was continued for another 2 h. The reaction mixture was then concentrated under vacuum. The residue was chromatographed by flash column chromatography and further purified by preparative TLC, eluting with methanol/dichloromethane/concentrated ammonium hydroxide (10/1/89, v/v), to afford the desired product 42 (0.031 g, 22%) as a yellow solid. mp 129-132° C. ¹H NMR (300 MHz, CD₃OD): δ 1.72 (m, 4H), 2.68 (t, 2H), 3.32 (m, 2H), 4.92 (s, 2H), 6.95 (d, 2H), 7.22 (d, 2H); m/z (APCI): 417 [C₁₈H₂₁ClN₈O₂+H]⁺.

### Example 11

### Synthesis of N-(3,5-diamino-6-chloropyrazine-2-carbonyl)-N′-(4-{4-[3-(2,3-dihydroxypropoxy)-2-hydroxypropoxy]phenyl}butyl)guanidine (PSA 15143)

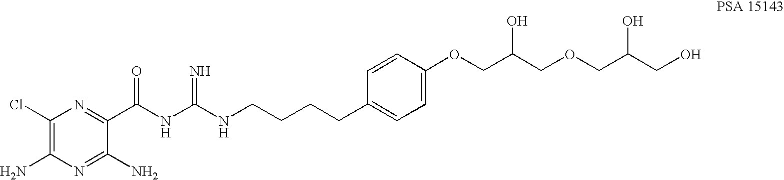

### (4-{4-[3-(2,3-Dihydroxypropoxy)-2-hydroxypropoxy]phenyl}butyl)carbamic acid benzyl ester (43)

A solution containing compound 1 (2.0 g, 6.68 mmol), triethylamine (0.093 mL, 0.668 mmol) and anhydrous ethanol (2.2 mL) was heated at 70° C. for 1 h. Oxiranylmethanol (0.5 mL, 6.68 mmol) was added every hour for a total of 4 h (the total amount of oxiranylmethanol added was 2.0 ml, 26.72 mmol). The reaction was concentrated under vacuum. The residue was chromatographed on silica gel with the elution of a mixture of methanol/dichloromethane (3:97, v/v) to provide 168 mg (4.6% yield) of the desired product 43. m/z (APCI): 448 [C₂₄H₃₃NO₇+H]⁺.

### 3-{3-[4-(4-Aminobutylphenoxy]-2-hydroxypropoxy}propane-1,2-diol (44)

A solution containing the compound 43 (0.15 g, 0.34 mmol) in ethanol (1.5 mL) was purged with nitrogen before and after the catalyst (0.15 g, 10% Pd/C, 50% wet) was added. The reaction mixture was placed under hydrogenation atmosphere for 45 min. The catalyst was vacuum filtered through diatomaceous earth and washed with ethanol (3×2 mL). The combined filtrates were concentrated under vacuum. The residue was chromatographed on silica gel, eluting with methanol/dichloromethane/concentrated ammonium (25/2.5/73.5, v/v), to afford the desired product 44 (0.053 g, 51% yield) as a colorless, viscous oil. ¹H NMR (300 MHz, CD₃OD): δ 1.52 (m, 4H), 2.55 (t, 2H), 2.65 (t, 2H), 3.61 (m, 10H), 6.85 (d, 2H), 7.09 (d, 2H). m/z (APCI): 314 [C₁₆H₂₇NO₅+H]⁺.

### N-(3,5-Diamino-6-chloropyrazine-2-carbonyl)-N′-(4-{4-[3-(2,3-dihydroxypropoxy)-2-hydroxypropoxy]phenyl}butyl)guanidine (45, PSA 15143)

Compound 44 (50 mg, 0.159 mmol) was dissolved in a mixture of absolute ethanol (0.5 mL) and triethylamine (0.076 mL, 0.541 mmol) at 65° C. over 15 min. To the solution was added 1-(3,5-diamino-6-chloropyrazine-2-carbonyl)-2-methylisothiourea hydriodide (74 mg, 0.191 mmol). The reaction mixture was stirred at the above temperature for an additional 50 min, cooled down to ambient temperature and subsequently concentrated under vacuum. The residue was chromatographed on silica gel, eluting with methanol/dichloromethane/concentrated ammonium hydroxide (10/1/40, v/v) to afford the desired product 45 (53 mg, 36% yield) as a yellow solid. mp 73-82° C. (decomposed). ¹H NMR (300 MHz, CD₃OD): δ 1.70 (m, 4H), 2.55 (m, 2H), 3.22 (m, 2H), 3.65 (m, 7H), 3.98 (m, 3H), 6.86 (d, 2H), 7.08 (d, 2H). m/z (APCI): 526 [C₂₂H₃₂ClN₇O₆+H]⁺.

### Example 12

Utilizing the procedures set forth above, the following Capped Pyrazinoylguanidine was prepared.

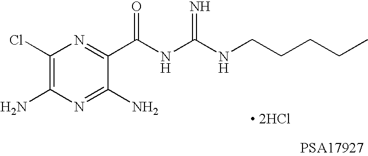

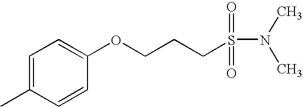

| TEST | RESULT/REFERENCE |
|---|---|
| Description | Yellow solid |
| Identification: | Consistent |
| 300 MHz ¹H NMR | |
| Spectrum (DMSO-d₆) | |
| Melting Point | 108-110° C. dec |
| HPLC Analysis | 96.5% (area percent), Polarity dC18 Column, |
| | Detector @ 220 nm |
| Miscellaneous Tests: | m/z 527 [C₂₁H₃₁ClN₈O₄S + H]⁺ |
| ESI Mass Spectrum | |

### Example 13

Utilizing the procedures set forth above, the following Capped Pyrazinoylguanidine was prepared.

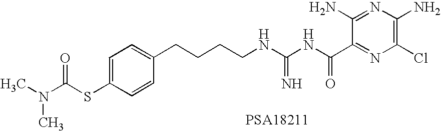

| TEST | RESULT/REFERENCE |
|---|---|
| Description | Yellow solid |
| Identification: | Consistent |
| 300 MHz ¹H NMR | |
| Spectrum (DMSO-d₆) | |
| Melting Point | 153-155° C. dec |
| HPLC Analysis | 96.3% (area percent), Polarity dC18 Column, |
| | Detector @ 220 nm |
| Miscellaneous Tests: | m/z 465 [C₁₉H₂₅ClN₈O₂S + H]⁺ |
| ESI Mass Spectrum | |

### Example 14

Utilizing the procedures set forth above, the following Capped Pyrazinoylguanidine was prepared.

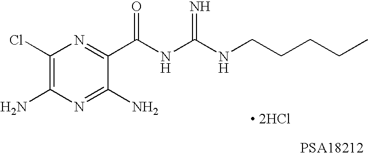

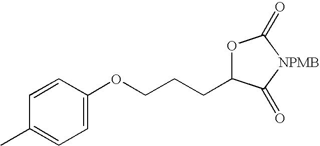

| TEST | RESULT/REFERENCE |
|---|---|
| Description | Yellow solid |
| Identification: | Consistent |
| 500 MHz ¹H NMR | |
| Spectrum (CD₃OD) | |
| Melting Point | 115-116° C. dec |
| HPLC Analysis | 97.1% (area percent), Polarity dC18 Column, |
| | Detector @ 220 nm |
| Miscellaneous Tests: | m/z 639 [C₃₀H₃₅ClN₈O₆ + H]⁺ |
| ESI Mass Spectrum | |

### Example 15

Utilizing the procedures set forth above, the following Capped Pyrazinoylguanidine was prepared.

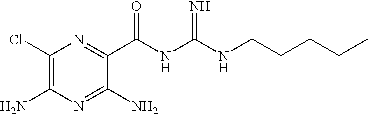

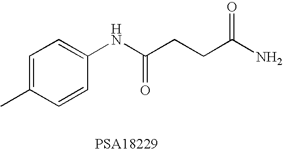

| TEST | RESULT/REFERENCE |
|---|---|
| Description | Yellow solid |
| Identification: | Consistent |
| 300 MHz ¹H NMR | |
| Spectrum (CD₃OD) | |
| Melting Point | 190-192° C. dec |
| HPLC Analysis | 97.9% (area percent), Polarity dC18 Column, |
| | Detector @ 220 nm |
| Miscellaneous Tests: | m/z 476 [C₂₀H₂₆ClN₉O₃ + H]⁺ |
| ESI Mass Spectrum | |

### Example 16

Utilizing the procedures set forth above, the following Capped Pyrazinoylguanidine was prepared.

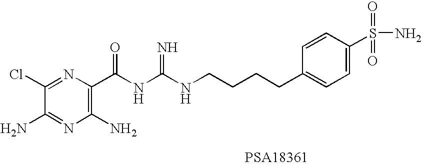

| TEST | RESULT/REFERENCE |
|---|---|
| Description | Yellow solid |
| Identification: | Consistent |
| 300 MHz ¹H NMR | |
| Spectrum (CD₃OD) | |
| Melting Point | 124-126° C. dec |
| HPLC Analysis | 95.2% (area percent), Polarity dC18 Column, |
| | Detector @ 220 nm |
| Miscellaneous Tests: | m/z 441 [C₁₆H₂₁ClN₈O₃S + H]⁺ |
| ESI Mass Spectrum | |

### Example 17

Utilizing the procedures set forth above, the following Capped Pyrazinoylguanidine was prepared.

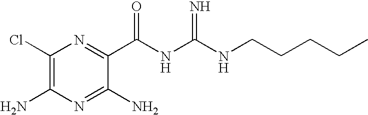

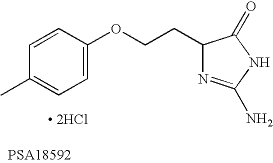

| TEST | RESULT/REFERENCE |
|---|---|
| Description | Yellow solid |
| Identification: | Consistent |
| 500 MHz ¹H NMR | |
| Spectrum (CD₃OD) | |
| Melting Point | 189° C. dec |
| HPLC Analysis | 95.0% (area percent), Polarity dC18 Column, |
| | Detector @ 220 nm |
| Miscellaneous Tests: | m/z 503 [C₂₁H₂₇ClN₁₀O₃ + H]⁺ |
| ESI Mass Spectrum | |

### Example 18

Utilizing the procedures set forth above, the following Capped Pyrazinoylguanidine was prepared.

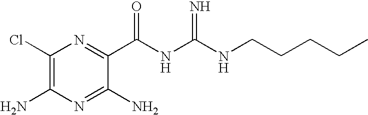

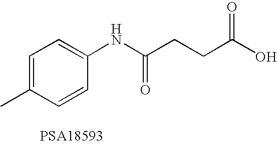

| TEST | RESULT/REFERENCE |
|---|---|
| Description | Pale yellow solid |
| Identification: | Consistent |
| 300 MHz ¹H NMR | |
| Spectrum (CD₃OD) | |
| Melting Point | 195-197° C. dec |
| HPLC Analysis | 97.4% (area percent), Polarity dC18 Column, |
| | Detector @ 220 nm |
| Miscellaneous Tests: | m/z 477 [C₂₀H₂₅ClN₈O₄ + H]⁺ |
| ESI Mass Spectrum | |

### Example 19

Utilizing the procedures set forth above, the following Capped Pyrazinoylguanidine was prepared.

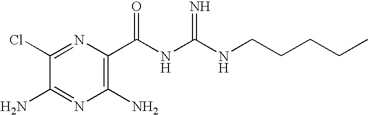

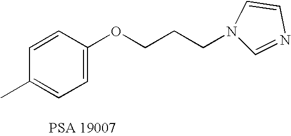

| TEST | RESULT/REFERENCE |
|---|---|
| Description | Yellow solid |
| Identification: | Consistent |
| 300 MHz ¹H NMR | |
| Spectrum (CD₃OD) | |
| Melting Point | 210-212° C. dec |
| HPLC Analysis | 95.5% (area percent), Polarity dC18 Column, |
| | Detector @ 220 nm |
| Miscellaneous Tests: | m/z 486 [C₂₂H₂₈ClN₉O₂ + H]⁺ |
| ESI Mass Spectrum | |

### Example 20

Utilizing the procedures set forth above, the following Capped Pyrazinoylguanidine was prepared.

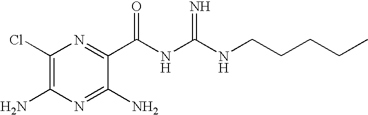

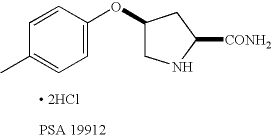

| TEST | RESULT/REFERENCE |
|---|---|
| Description | Yellow solid |
| Identification: | Consistent |
| 300 MHz ¹H NMR | |
| Spectrum (CD₃OD) | |
| Optical Rotation | [α]²⁵_{D} − 7.8° (c 0.30, Methanol) |
| Melting Point | 178-180° C. dec |
| HPLC Analysis | 97.0% (area percent), Polarity dC18 Column, |
| | Detector @ 220 nm |
| Miscellaneous Tests: | m/z 490 [C₂₁H₂₈ClN₉O₃ + H]⁺ |
| ESI Mass Spectrum | |

### Example 21

Utilizing the procedures set forth above, the following Capped Pyrazinoylguanidine was prepared.

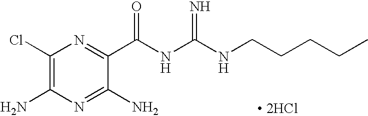

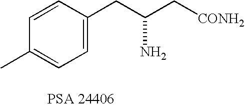

| TEST | RESULT/REFERENCE |
|---|---|
| Description | Yellow solid |
| Identification: | Consistent |
| 300 MHz ¹H NMR | |
| Spectrum (CD₃OD) | |
| Optical Rotation | [α]²⁵_{D} + 0.5° (c 0.35, Methanol) |
| Melting Point | 215° C. dec |
| HPLC Analysis | 96.1% (area percent), Polarity dC18 Column, |
| | Detector @ 220 nm |
| Miscellaneous Tests: | m/z 462 [C₂₀H₂₈ClN₉O₂ + H]⁺ |
| ESI Mass Spectrum | |

### Example 22

Utilizing the procedures set forth above, the following Capped Pyrazinoylguanidine was prepared.

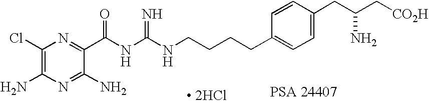

| TEST | RESULT/REFERENCE |
|---|---|
| Description | Yellow solid |
| Identification: | Consistent |
| 300 MHz ¹H NMR | |
| Spectrum (CD₃OD) | |
| Optical Rotation | [α]²⁵_{D} + 4.1+ (c 0.30, Methanol) |
| Melting Point | 230° C. dec |
| HPLC Analysis | 95.3% (area percent), Polarity dC18 Column, |
| | Detector @220 nm |
| Miscellaneous Tests: | m/z 463 [C₂₀H₂₇ClN₈O₃ + H]⁺ |
| ESI Mass Spectrum | |

### Example 23

Sodium Channel Blocking Activity of Selected Capped Pyrazinoylguanidines.

| | | | Fold Amiloride** | |
|---|---|---|---|---|
| PSA | EC₅₀(nM) | | (PSA 4022 = 100) | |
| 15143 | 7 ± 3 | (n = 3) | 107 ± 11 | (n = 3) |
| 16208 | 11 ± 4 | (n = 6) | 52 ± 21 | (n = 6) |
| 16314 | 13 ± 2 | (n = 4) | 41 ± 6 | (n = 4) |
| 16313 | 15 ± 4 | (n = 4) | 41 ± 7 | (n = 4) |
| 16437 | 13 ± 7 | (n = 7) | 77 ± 53 | (n = 7) |
| 17482 | 16 ± 4 | (n = 3) | 39 ± 6 | (n = 3) |
| 17846 | 11 ± 6 | (n = 4) | 104 ± 49 | (n = 4) |
| 17926 | 25 ± 9 | (n = 6) | 29 ± 12 | (n = 6) |
| 17927 | 13 ± 4 | (n = 3) | 83 ± 26 | (n = 3) |
| 18211 | 10 ± 4 | (n = 3) | 112 ± 52 | (n = 2) |
| 18212 | 27 ± 17 | (n = 4) | 32 ± 16 | (n = 4) |
| 18229 | 15 ± 6 | (n = 3) | 49 ± 15 | (n = 3) |
| 18361 | 11 ± 4 | (n = 3) | 76 ± 25 | (n = 3) |
| 18592 | 8 ± 4 | (n = 2) | 136 ± 58 | (n = 2) |
| 18593 | 48 ± 16 | (n = 6) | 13 ± 4 | (n = 4) |
| 19007 | 18 ± 13 | (n = 4) | 42 ± 17 | (n = 4) |
| 19008 | 9 ± 1 | (n = 4) | 54 ± 6 | (n = 4) |
| 19912 | 26 ± 8 | (n = 4) | 32 ± 10 | (n = 4) |
| 23022 | 12 ± 3 | (n = 4) | 79 ± 15 | (n = 4) |
| 24406 | 8 ± 3 | (n = 6) | 107 ± 38 | (n = 6) |
| 24407 | 32 ± 11 | (n = 10) | 23 ± 4 | (n = 10) |
| 24851 | 28 ± 13 | (n = 8) | 25 ± 10 | (n = 8) |

**Relative potency for PSA 4022 = 100 using EC₅₀ from PSA 4022 in same run

The following examples depict the synthesis of compounds according to Formula II.

### FORMULA II EXAMPLES

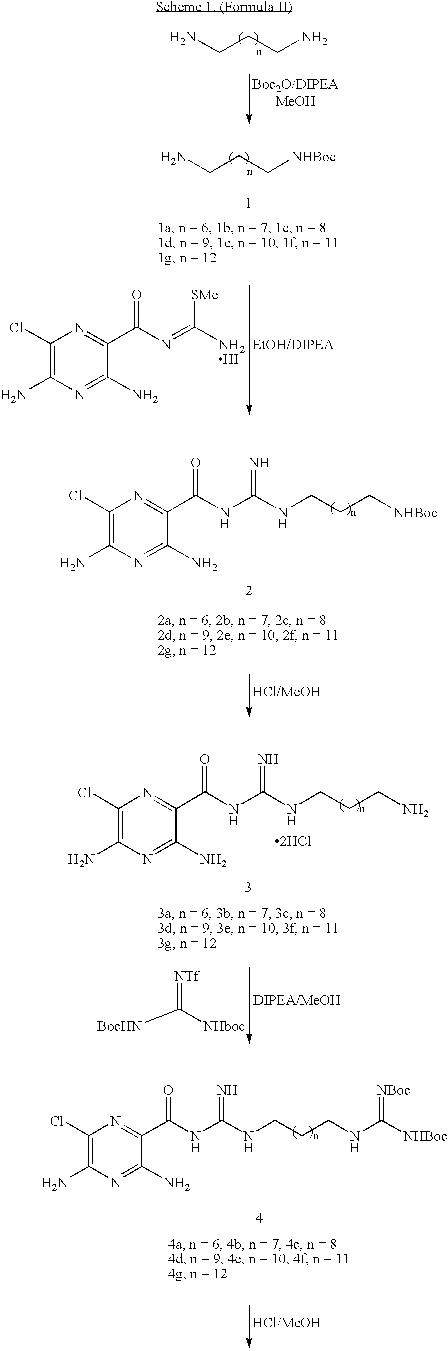 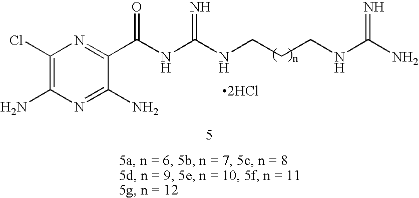 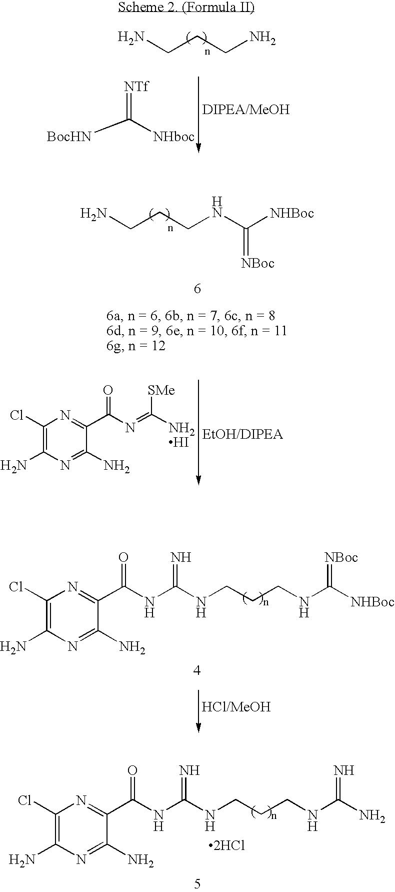 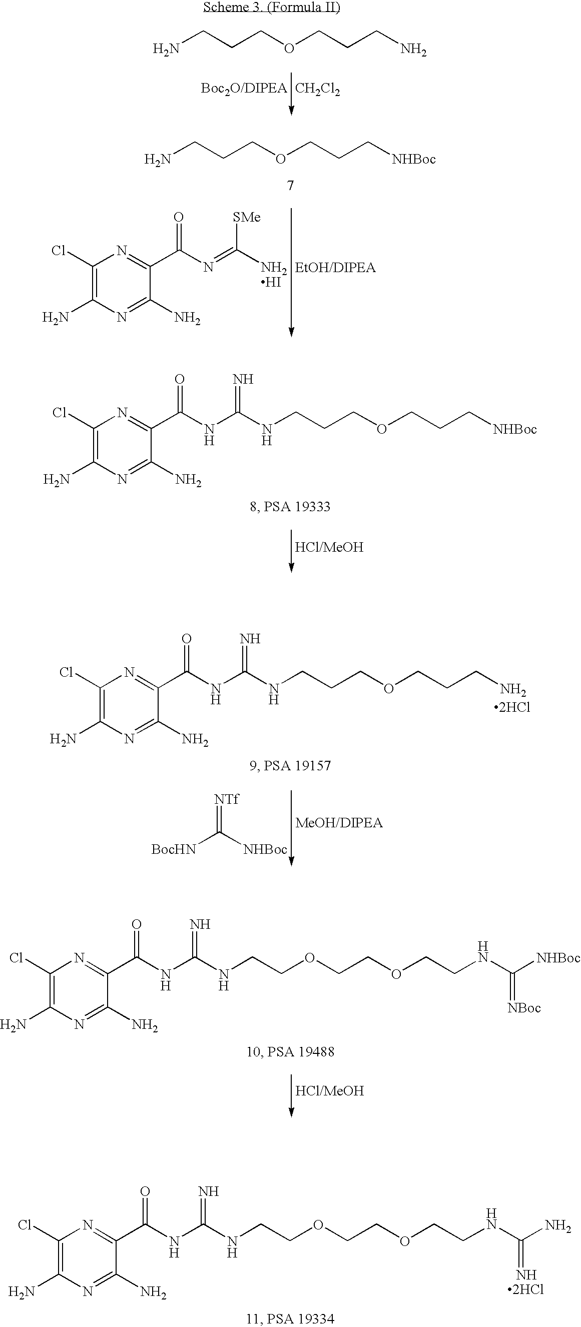 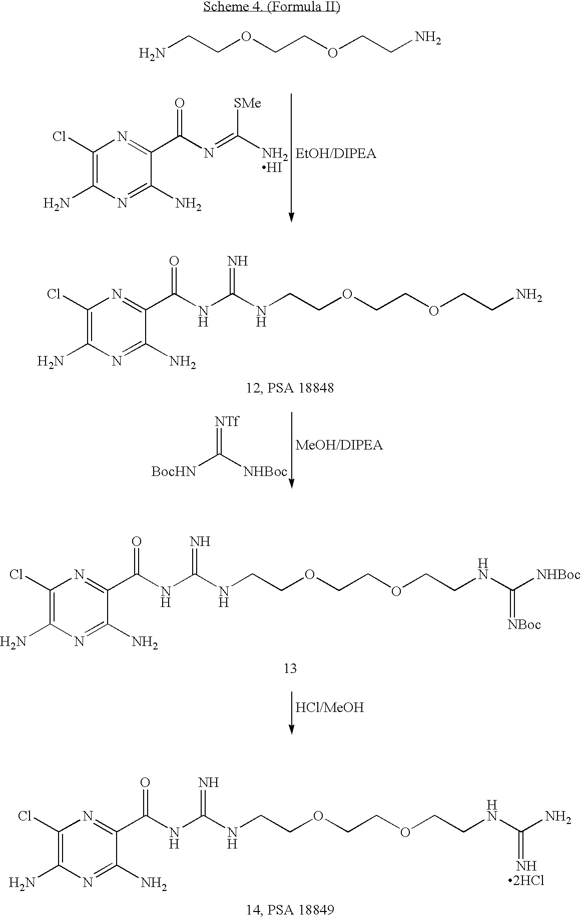 General Procedures

Method A. Mono-Protection of Symmetrical Diamine by Boc-Protecting Group

The diamine was dissolved in anhydrous methanol. To the solution was added Hunig's base (DIPEA, 3 equiv). The newly resulting solution was stirred at room temperature for 30 min. To the reaction mixture was slowly added (over 2 to 4 hours) a solution of Boc₂O (1 equiv) dissolved in anhydrous methanol. After the addition, the reaction mixture was stirred for an additional 2 hours, then quenched with water. The product was extracted with dichloromethane. The combined extracts were washed with brine, dried over anhydrous Na₂SO₄ and concentrated. The residue was chromatographed on silica gel eluting with a mixture of methanol and dichloromethane. The fractions containing the desired product were collected and concentrated under vacuum. The product was spectroscopically characterized.

Method B. Removal of Boc-Protecting Group from Amino or Guanidino Group

The compound containing Boc-protected amino or guanidino group was dissolved in methanol. The solution was then treated with concentrated HCl (12 N) at room temperature for 1 to 2 hours. All liquid in the reaction mixture was then completely removed under vacuum. The resulting residue was further dried under vacuum and generally directly used in the next step without purification.

Method C. Guanylation of Free Amine by Reaction with (tert-butoxycarbonylamino-trifluoromethanesulfonyliminomethyl)carbamic acid tert-butyl ester (Goodman's Reagent)

To a solution containing the free amine dissolved in anhydrous methanol was added Hunig's base (DIPEA, 3 equiv). The newly resulting solution was stirred at room temperature for 30 min before the Goodman's reagent was added (1.5 equiv). The stirring was continued for an additional 3 to 5 hours. The reaction mixture was concentrated. The resulting residue was chromatographed on silica gel eluting with a mixture of dichloromethane, methanol, and concentrated ammonium hydroxide (CMA). The fractions containing the desired product were collected and concentrated. The product was characterized by spectroscopic methods.

Method D. Coupling of Un-Protected Amine with 1-(3,5-diamino-6-chloropyrazine-2-carbonyl)-2-methylisothiourea hydriodide (Cragoe Compound)

The un-protected amine was dissolved in anhydrous ethanol. To the solution was added Hunig's base (DIPEA, 3 equiv). The newly resulting solution was heated at 65° C. for 15 min. The Cragoe compound (1.2 equiv) was then added. The reaction mixture was stirred at 65° C. for an additional 2 to 3 hours, and then cooled to room temperature before it was concentrated under vacuum. The resulting residue was chromatographed on silica gel eluting with CMA. The appropriate fractions were collected and concentrated under vacuum. The desired product (typically a yellow solid) was characterized by spectroscopic methods.

### Example 1

### Synthesis of N-(6-aminohexyl)-N′-(3,5-diamino-6-chloropyrazine-2-carbonyl)guanidine dihydrochloride (PSA 18706)

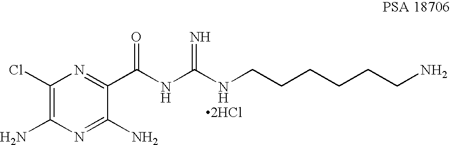

### {6-[N′-(3,5-Diamino-6-chloropyrazine-2-carbonyl)guanidino]hexyl}carbamic acid tert-butyl ester (2a)

Compound 2a was synthesized from 1a, 6-aminohexylcarbamic acid tert-butyl ester (Scheme 1), in 90% yield using method D. ¹H NMR (300 MHz, CD₃OD): δ 1.42 (s, 9H), 1.46-1.65 (m, 8H), 3.04 (t, 2H), 3.22 (t, 2H). m/z (APCI): 429 [C₁₇H₂₉ClN₈O₃+H]⁺.

### N-(6-Aminohexyl)-N′-(3,5-diamino-6-chloropyrazine-2-carbonyl)guanidine dihydrochloride (3a, PSA 18706)

Compound 3a was synthesized from compound 2a in 34% yield using method B. mp>240° C. ¹H NMR (300 MHz, CD₃OD): δ 1.42-1.54 (m, 4H), 1.65-1.78 (m, 4H), 2.94 (t, 2H), 3.34 (t, 2H). m/z (APCI): 329 [C₁₂H₂₁ClN₈O+H]⁺.

### Example 2

### Synthesis of N-(7-aminoheptyl)-N′-(3,5-diamino-6-chloropyrazine-2-carbonyl)guanidine (PSA 18705)

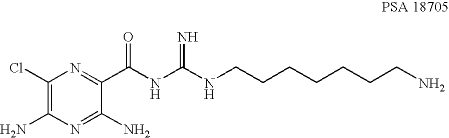

Compound 3b (PSA 18705) was synthesized from heptane-1,7-diamine in 65% yield using method D. mp 185-187° C. (decomposed). ¹H NMR (300 MHZ, CD₃OD): δ 1.40-1.55 (m, 6H), 1.58-1.76 (m, 4H), 2.80 (t, 2H), 3.30 (m, 2H). m/z (ESI): 343 [C₁₃H₂₃ClN₈O+H]⁺.

### Example 3

### Synthesis of N-{7-[N′-(3,5-diamino-6-chloropyrazine-2-carbonyl)guanidino]heptyl}guanidine dihydrochloride (PSA 19155)

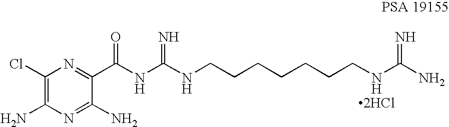

### N-(7-Aminoheptyl)-[N′,N′-bis-(tert-butoxycarbonyl)]guanidine (6b)

Compound 6b was synthesized from heptane-1,7-diamine (Scheme 2) in 43% yield using method C. ¹H NMR (300 MHz, CDCl₃): δ 1.44-1.50 (m, 10H), 1.55 (s, 18H), 1.84 (t, 2H), 2.78 (t, 2H), 3.46 (t, 2H). m/z (ESI): 373 [C₁₈H₃₆N₄O₄+H]⁺.

### N-{7-[N′-(3,5-Diamino-6-chloropyrazine-2-carbonyl)guanidino]heptyl}guanidine dihydrochloride (5b, PSA 19155)

Compound 6b was reacted with the Cragoe compound according to method D (Scheme 2). The product of the reaction, after chromatographic purification, was directly treated with concentrated HCl using method B to afford the desired compound 5b in 17% overall yield. mp 140-142° C. ¹H NMR (300 MHz, CD₃OD): δ 1.42-1.54 (m, 6H), 1.60-1.82 (m, 4H), 3.18 (t, 2H), 3.34 (m, 2H). m/z (ESI): 385 [C₁₄H₂₅ClN₁₀O+H]⁺.

### Example 4

### Synthesis of {8-[N′-(3,5-diamino-6-chloropyrazine-2-carbonyl)guanidino]octyl}carbamic acid tert-butyl ester (PSA 19156)

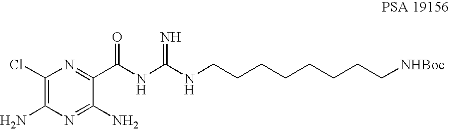

Octane-1,8-diamine was mono-protected by Boc-protecting group using method A (Scheme 1). The product from this step was directly reacted with the Cragoe compound using method D, which afforded the desired product 2c (PSA 19156) in 81% yield. mp 189-191° C. ¹H NMR (300 MHz, CD₃OD): δ 1.38-1.56 (m, 19H), 1.70 (m, 2H), 3.02 (t, 2H), 3.24 (t, 2H). m/z (ESI): 457 [C₁₉H₃₃ClN₈O₃+H]⁺.

### Example 5

### Synthesis of N-(8-aminooctyl)-N′-(3,5-diamino-6-chloropyrazine-2-carbonyl)-guanidine dihydrochloride (PSA 19336)

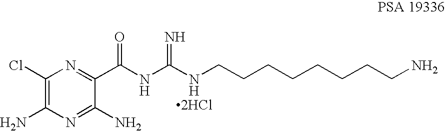

Compound 3c (PSA 19336) was synthesized from 2c using method B. mp 253-255° C. ¹H NMR (300 MHz, CD₃OD) 6 1.40 (m, 8H), 1.66 (m, 4H), 2.90 (m, 2H), 3.32 (m, 2H). m/z (ESI): 357 [C₁₄H₂₅ClN₈O+H]⁺.

### Example 6

### Synthesis of N-{8-[N′-(3,5-diamino-6-chloropyrazine-2-carbonyl)guanidino]octyl}{N″,N′″-bis-(tert-butoxycarbonyl)}guanidine (PSA 19486)

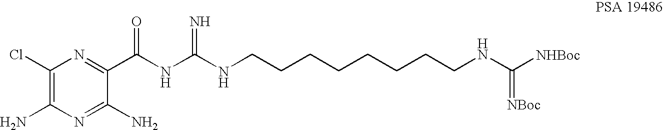

Compound 4c (PSA 19486) was synthesized from 3c in 52% yield using method C. mp 208-210° C. (decomposed). ¹H NMR (300 MHz, CD₃OD) δ 1.33-1.72 (m, 30H), 3.18-3.39 (m, 4H). m/z (ESI): 599 [C₂₅H₄₃ClN₁₀O₅+H]⁺.

### Example 7

### Synthesis of N-{8-[N′-(3,5-diamino-6-chloropyrazine-2-carbonyl)guanidino]octyl}-guanidine dihydrochloride (PSA 19604)

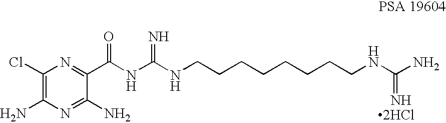

Compound 5c (PSA 19604) was synthesized from 4c in quantitative yield using method B. mp 130-132° C. ¹H NMR (300 MHz, CD₃OD) δ 1.33-1.72 (m, 12H), 3.18-3.39 (m, 4H). m/z (ESI): 399 [C₁₅H₂₇ClN₁₀O+H]⁺.

### Example 8

### Synthesis of {9-[N′-(3,5-diamino-6-chloropyrazine-2-carbonyl)guanidino]nonyl}-carbamic acid tert-butyl ester (PSA 19484)

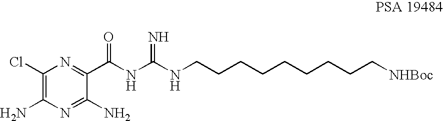

Compound 2d (PSA 19484) was synthesized in a similar method to compound 2c (PSA 19156). mp 187-189° C. ¹H NMR (500 MHz, CD₃OD) δ 1.35 (m, 12H), 1.41 (s, 9H), 1.60 (m, 2H), 3.00 (m, 2H), 3.20 (m, 2H). m/z (ESI): 471 [C₂₀H₃₅ClN₈O₃+H]⁺.

### Example 9

### Synthesis of N-(9-aminononyl)-N′-(3,5-diamino-6-chloropyrazine-2-carbonyl)-guanidine dihydrochloride (PSA 19335)

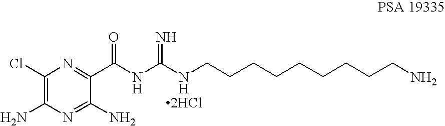

Compound 3d (PSA 19335) was synthesized in quantitative yield from 2d (PSA 19484) using method B. mp 155-157° C. (decomposed). ¹H NMR (300 MHz, CD₃OD) δ 1.40 (m, 10H), 1.70 (m, 4H), 2.90 (m, 2H), 3.32 (m, 2H). m/z (ESI): 357 [C₁₅H₂₇ClN₈O+H]⁺.

### Example 11

### Synthesis of N-{9-[N′-(3,5-diamino-6-chloropyrazine-2-carbonyl)guanidino]nonyl}guanidine dihydrochloride (PSA 19006)

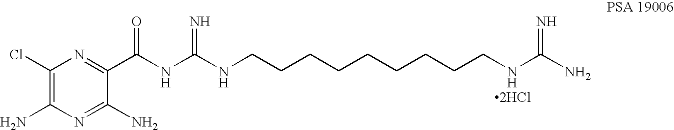

Compound 5d (PSA 19006) was synthesized similarly to compound 5c (PSA 19155). mp 178-180° C. ¹H NMR (300 MHz, CD₃OD): δ 1.44-1.54 (m, 10H), 1.58-1.80 (m, 4H), 3.20 (t, 2H), 3.34 (m, 2H). m/z (ESI): 413 [C₁₆H₂₉ClN₁₀O+H]⁺.

### Example 12

### Synthesis of {10-[N′-(3,5-diamino-6-chloropyrazine-2-carbonyl)guanidino]decyl}-carbamic acid tert-butyl ester (PSA 19485)

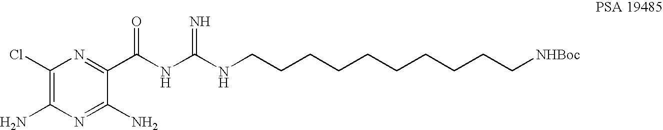

Compound 2e (PSA 19485) was synthesized in a similar method to compound 2c. mp 186-188° C. ¹H NMR (300 MHz, CD₃OD) δ 1.29-1.51 (m, 23H), 1.59-1.70 (m, 2H), 3.02 (t, 2H), 3.19-3.28 (m, 2H). m/z (ESI): 485 [C₂₁H₃₇ClN₈O₃+H]⁺.

### Example 13

### Synthesis of N-(10-aminodecyl)-N′-(3,5-diamino-6-chloropyrazine-2-carbonyl)-guanidine dihydrochloride (PSA 19487)

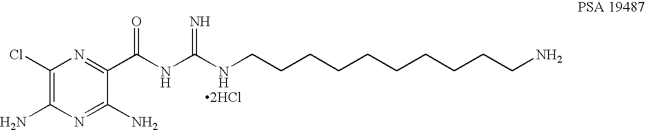

Compound 3e (PSA 19487) was synthesized from compound 2e using method B. mp 168-170° C. ¹H NMR (300 MHz, CD₃OD) δ 1.41(m, 12H), 1.57-1.79 (m, 4H), 2.84-2.99 (m, 2H), 3.34-3.40 (m, 2H); m/z (ESI): 385 [C₁₆H₂₉ClN₈O+H]⁺.

### Example 14

### Synthesis of N-{10-[N′-(3,5-diamino-6-chloropyrazine-2-carbonyl)guanidino]-decyl}guanidine dihydrochloride (PSA 23608)

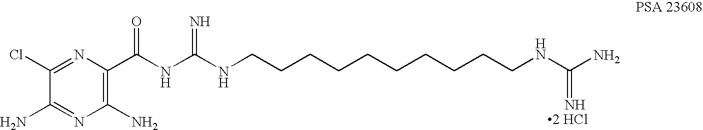

Compound 3e (PSA 19487) was reacted with the Goodman's reagent (Scheme 1) according to method C. The product of the reaction, after chromatographic purification, was directly treated with concentrated HCl using method B to afford the desired product 5e (PSA 23608). mp 156-158° C. ¹H NMR (500 MHz, CD₃OD) δ 1.31-1.48 (m, 12H), 1.55-1.62 (m, 2H), 1.65-1.76 (m, 2H), 3.11-3.19 (m, 2H), 3.34-3.38 (m, 2H). m/z (ESI): 427 [C₁₇H₃₁ClN₁₀O+H]⁺.

### Example 15

### Synthesis of {11-[N′-(3,5-diamino-6-chloropyrazine-2-carbonyl)guanidino]undecyl}carbamic acid tert-butyl ester (PSA 23777)

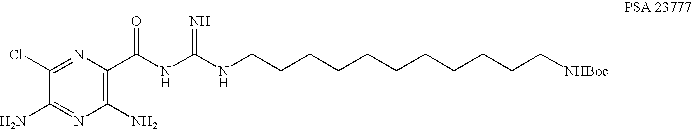

Compound 2f (PSA 23777) was synthesized in a similar method to compound 2c (PSA 19156). mp 82-84° C. ¹H NMR (500 MHz, CD₃OD) δ 1.27 (s, 12H), 1.45 (s, 13H), 1.65 (m, 2), 2.95 (m, 2H), 3.21 (m, 2H). m/z (APCI): 499 [C₂₂H₃₉ClN₈O₃+H]⁺.

### Example 16

### Synthesis of N-(11-aminoundecyl)-N′-(3,5-diamino-6-chloropyrazine-2-carbonyl)guanidine dihydrochloride (PSA 23682)

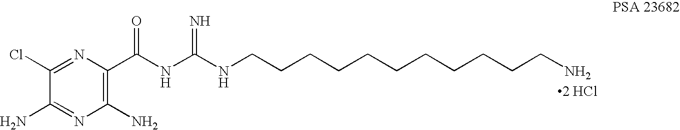

Compound 3f (PSA 23682) was synthesized from 2f using method B. mp 220-222° C. ¹H NMR (500 MHz, CD₃OD) δ 1.35 (m, 14H), 1.65 (m, 4H), 2.91 (m, 2H), 3.31 (m, 2H). m/z (APCI): 399 [C₁₇H₃₁ClN₈O+H]⁺.

### Example 17

### Synthesis of N-{11-[N′-(3,5-diamino-6-chloropyrazine-2-carbonyl)guanidino]-undecyl}guanidine dihydrochloride (PSA 23991)

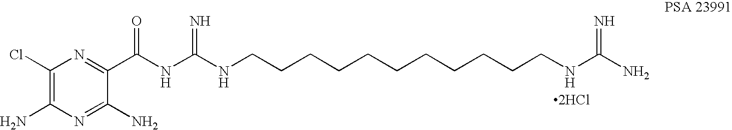

Compound 5f (PSA 23991) was synthesized in a similar manner to compound 5e. mp 151-153° C. ¹H NMR (300 MHz, DMSO-d₆) δ 1.27 (m, 18H), 1.45 (m, 2H), 1.55 (m, 2H), 3.07 (m, 2H), 3.27 (m, 2H), 7.43 (m, 2H), 7.66 (m, 1H), 8.78 (br, 1H), 8.94(br, 1H), 9.25 (br, 1H), 10.5 (br, 1H). m/z (APCI): 441 [C₁₈H₃₃ClN₁₀O+H]⁺.

### Example 18

### Synthesis of {12-[N′-(3,5-diamino-6-chloropyrazine-2-carbonyl)guanidino]dodecyl}-carbamic acid tert-butyl ester (PSA 23776)

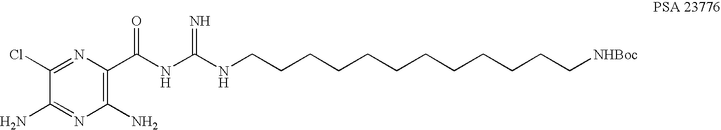

Compound 2g (PSA 23776) was synthesized similarly to compound 2c. mp 154-156° C. ¹H NMR (500 MHz, CD₃OD) δ 1.25 (m, 14H), 1.47 (m, 13H), 1.65 (m, 2H), 2.98 (m, 2H), 3.21 (m, 2H). m/z (APCI): 513 [C₂₃H₄₁ClN₈O₃+H]⁺.

### Example 19

### Synthesis of N-(12-aminododecyl)-N′-(3,5-diamino-6-chloropyrazine-2-carbonyl)-guanidine dihydrochloride (PSA 23609)

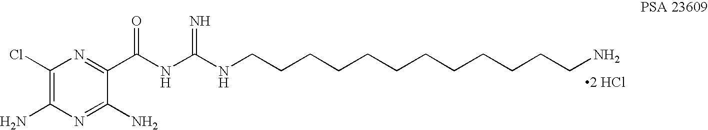

Compound 3g (PSA 23609) was synthesized from compound 2g using method B. mp 235-237° C. ¹H NMR (500 MHz, CD₃OD) δ 1.35 (m, 16H), 1.65 (m, 4H), 2.89 (m, 2H), 3.31 (m, 2H). m/z (ESI): 413 [C₁₈H₃₃ClN₈O+H]⁺.

### Example 20

### Synthesis of N-{12-[N′-(3,5-diamino-6-chloropyrazine-2-carbonyl)guanidino]dodecyl)guanidine dihydrochloride (PSA 23683)

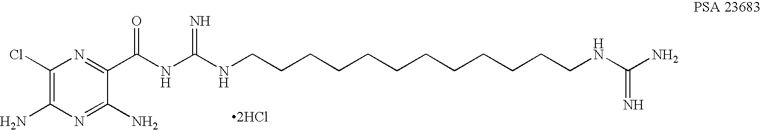

Compound 5g (PSA 23683) was synthesized from compound 3g in a similar method to 5b. mp 145-147° C. ¹H NMR (500 MHz, CD₃OD) δ 1.30-1.48 (m, 16H), 1.64 (t, 2H), 1.75 (t, 2H), 3.18 (t, 2H), 3.35 (m, 2H). m/z (APCI): 455 [C₁₉H₃₅ClN₁₀O+H]⁺.

### Example 21

### Synthesis of (3-{3-[N′-(3,5-diamino-6-chloropyrazine-2-carbonyl)guanidino]propoxy}propyl])carbamic acid tert-butyl ester (PSA19333)

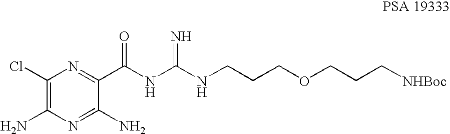

### [3-(3-Aminopropoxy)propyl]carbamic acid tert-butyl ester (7) Compound 7 was synthesized from 3-(3-aminopropoxy)propylamine (Scheme 3) using method A. ¹H NMR (300 MHz, CDCl₃) δ 1.40 (m, 2H), 1.44 (s, 9H), 1.74 (m, 4H), 2.81 (m, 2H), 3.23 (m, 2H), 3.48 (m, 4H), 5.03 (br s, 1H).

### (3-{3-[N′-(3,5-Diamino-6-chloropyrazine-2-carbonyl)guanidino]propoxy}propyl)-carbamic acid tert-butyl ester (8, PSA 19333)

Compound 8 was synthesized from compound 7 using method D. mp 62-65° C. (decomposed). ¹H NMR (300 MHz, CD₃OD) δ 1.40 (s, 9H), 1.80 (m, 4H), 3.12 (m, 2H), 3.32 (m, 2H), 3.52 (m, 4H). m/z (ESI): 445 [C₁₇H₂₉ClN₈O₄+H]⁺.

### Example 22

### Synthesis of N-[3-(3-aminopropoxy)propyl]-N′-(3,5-diamino-6-chloropyrazine-2-carbonyl)guanidine dihydrochloride (PSA19157)

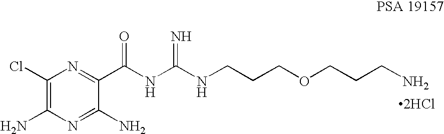

Compound 9 (PSA 19157) was synthesized from compound 8 (PSA 19333) using method B. mp 164-166° C. (decomposed). ¹H NMR (300 MHz, CD₃OD) δ 1.95 (m, 4H), 3.05 (m, 2H), 3.48 (m, 2H), 3.60 (m, 4H). m/z (ESI): 345 [C₁₂H₂₁ClN₈O₂+H]⁺.

### Example 23

### Synthesis of N-(3,5-diamino-6-chloropyrazine-2-carbonyl)-N′-[3-(3-{N″,N′″-bis-(tert-butoxycarbonyl)guanidino}propoxy)propyl]guanidine (PSA 19488)

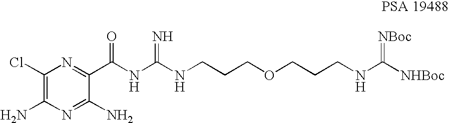

Compound 10 (PSA 19488) was synthesized from compound 9 (PSA 19157) using method C. mp 89-93° C. ¹H NMR (300 MHz, CD₃OD) δ 1.46 (s, 9H), 1.50 (s, 9H), 1.90 (m, 4H), 3.40 (m, 4H), 3.55 (m, 4H). m/z (ESI): 587 [C₂₃H₃₉ClN₁₀O₆+H]⁺.

### Example 24

### Synthesis of N-(3,5-diamino-6-chloropyrazine-2-carbonyl)-N′-[3-(3-guanidino-propoxy)propyl]guanidine dihydrochloride (PSA 19334)

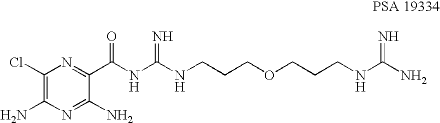

Compound 11 (PSA 19334) was synthesized from compound 10 (PSA 19488) using method B. mp 72-75° C. (decomposed). ¹H NMR (300 MHz, CD₃OD) δ 1.91 (m, 4H), 3.30 (m, 2H), 3.50 (m, 2H), 3.60 (m, 4H). m/z (ESI): 387 [C₁₃H₂₃ClN₁₀O₂+H]⁺.

### Example 25

### Synthesis of N-{2-[2-(2-aminoethoxy)ethoxy]ethyl}-N′-(3,5-diamino-6-chloro-pyrazine-2-carbonyl)guanidine (PSA 18848)

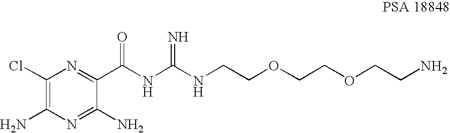

Compound 12 (PSA 18848) was synthesized from 2-[2-(2-aminoethoxy)ethoxy]-ethylamine (Scheme 4) in 86% yield using method D. mp 87-90° C. ¹H NMR (300 MHz, CD₃OD): δ 2.84 (t, 2H), 3.45 (t, 2H), 3.54-3.66 (m, 8H). m/z (APCI): 361 [C₁₂H₂₁ClN₈O₃+H]⁺.

### Example 26

### Synthesis of N-(3,5-diamino-6-chloropyrazine-2-carbonyl)-N′-{2-[2-(2-guanidinoethoxy)ethoxy]ethyl}guanidine dihydrochloride (PSA 18849)

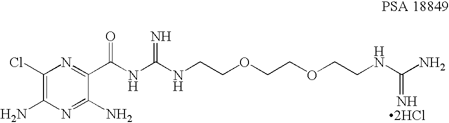

Compound 14 (PSA 18849) was synthesized from compound 12 by a similar method used to prepared compound 5e. mp 108-112° C. (decomposed). ¹H NMR (300 MHz, CD₃OD): δ 1.38 (t, 2H), 3.38 (t, 2H), 3.57 (t, 2H), 3.67 (t, 2H), 3.75 (m, 4H). m/z (APCI): 403 [C₁₃H₂₃ClN₁₀O₃+H]⁺.

### Example 27

Sodium Channel Blocking Activity of Selected Alaphatic Pyrazinoylguanidines.

| | | | Fold Amiloride** | |
|---|---|---|---|---|
| PSA | EC₅₀(nM) | | (PSA 4022 = 100) | |
| 18705 | 99 ± 31 | (n = 4) | 8 ± 2 | (n = 4) |
| 18706 | 254 ± 118 | (n = 4) | 4 ± 1 | (n = 4) |
| 19006 | 60 ± 15 | (n = 3) | 11 ± 2 | (n = 3) |
| 19155 | 81 ± 45 | (n = 3) | 8 ± 7 | (n = 3) |
| 19156 | 46 ± 20 | (n = 2) | 18 ± 2 | (n = 2) |
| 19333 | 81 ± 8 | (n = 4) | 7 ± 2 | (n = 4) |
| 19335 | 36 ± 7 | (n = 4) | 19 ± 7 | (n = 4) |
| 19336 | 76 ± 18 | (n = 4) | 12 ± 3 | (n = 3) |
| 19484 | 66 | (n = 1) | 12 | (n = 1) |
| 19487 | 25 ± 11 | (n = 4) | 37 ± 1 | (n = 4) |
| 19604 | 25 ± 27 | (n = 4) | 63 ± 61 | (n = 4) |
| 23608 | 17 ± 8 | (n = 2) | 41 ± 29 | (n = 2) |
| 23609 | 13 ± 7 | (n = 4) | 66 ± 36 | (n = 4) |
| 23682 | 12 ± 3 | (n = 3) | 51 ± 15 | (n = 3) |
| 23683 | 41 ± 68 | (n = 6) | 68 ± 48 | (n = 6) |
| 23776 | 75 | (n = 1) | 7 | (n = 1) |
| 23991 | 64 ± 77 | (n = 4) | 20 ± 12 | (n = 4) |

**Relative potency for PSA 4022 = 100 using EC₅₀ from PSA 4022 in same run

The following examples depict the synthesis of compounds according to Formula III.

### FORMULA III EXAMPLES

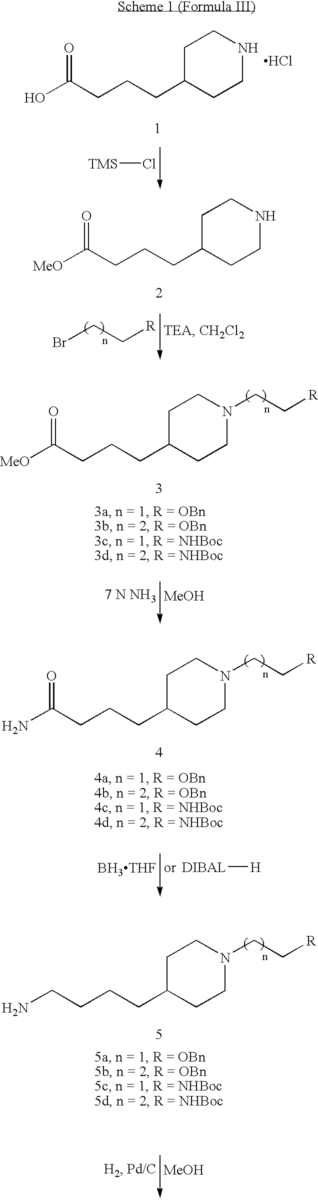

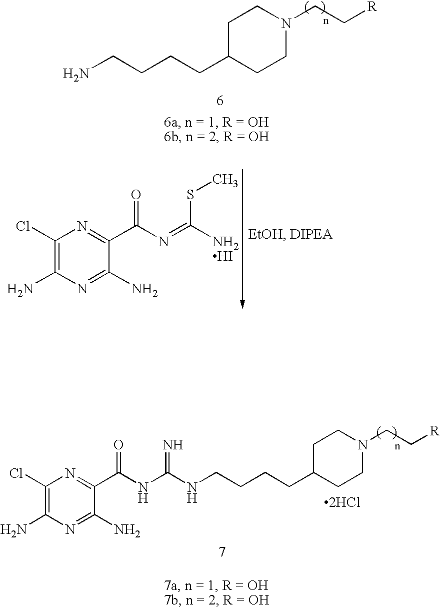

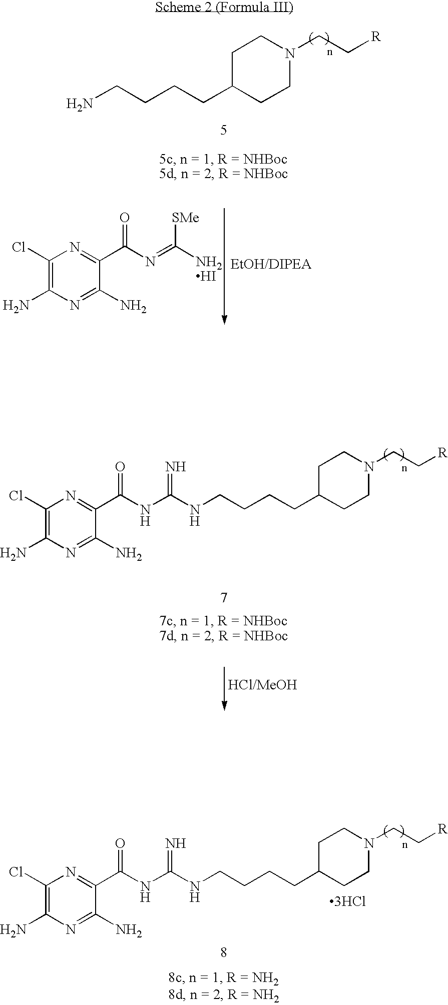

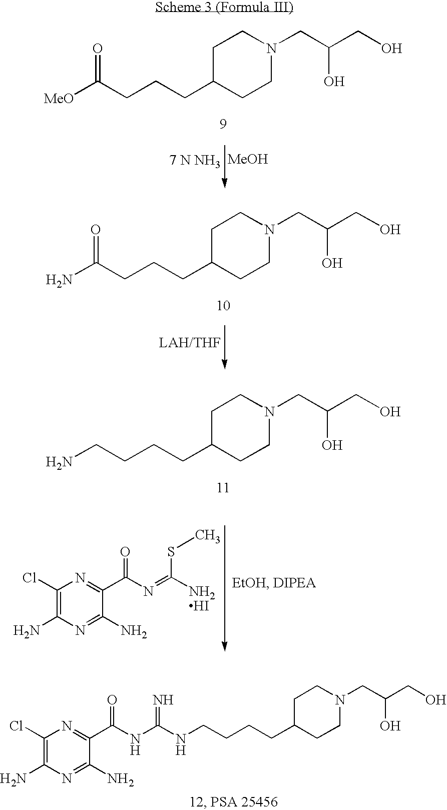

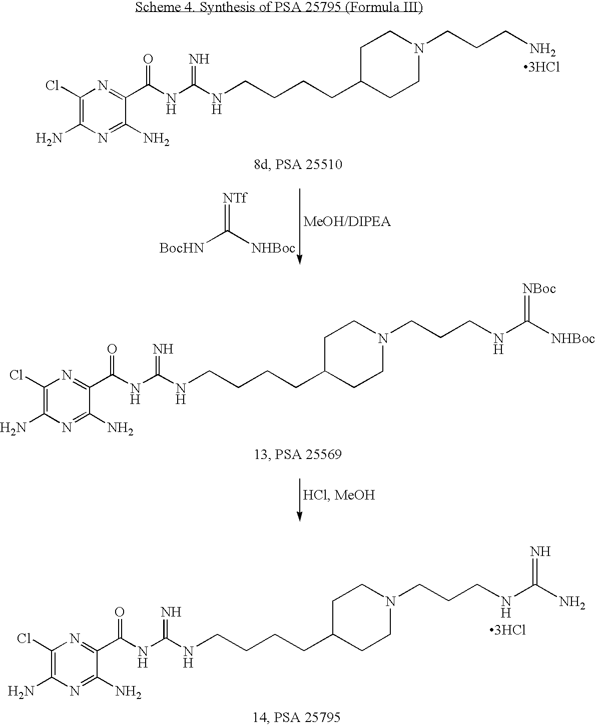

### Example 1

### Synthesis of N-(3,5-diamino-6-chloropyrazine-2-carbonyl)-N′-{4-[1-(2-hydroxyethyl)piperidin-4-yl]butyl}guanidine dihydrochloride (PSA 25193)

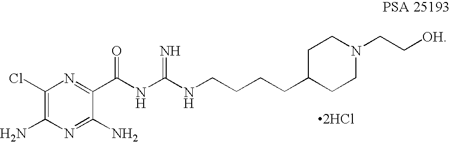

### 4-(Piperidin-4-yl)butyric acid methyl ester (2)

A solution of 1 (2.00 g, 9.50 mmol) and chlorotrimethylsilane (2.30 g, 20.1 mmol) in methanol (30 mL) was stirred at room temperature overnight (Scheme 1). After that, the solvent was removed under reduced pressure and the residue was purified by Flash™ chromatography (BIOTAGE, Inc) (9:1 dichloromethane/methanol, v/v) to provide 2 (1.73 g, 98%) as a light yellow solid.

¹H NMR (300 MHz, CD₃OD) δ 1.39 (m, 4H), 1.66 (m, 3H), 1.95 (d, 2H), 2.39 (m, 2H), 3.02 (m, 2H), 3.40 (m, 2H), 3.69 (s, 3H). m/z (ESI): 186 [C₁₀H₁₉NO₂+H]⁺.

### 4-[1-(2-Benzyloxyethyl)piperidin-4-yl]butyric acid methyl ester (3a)

A solution of 2 (2.00 g, 10.8 mmol), (2-bromoethoxymethyl)benzene (2.31 g, 10.8 mmol), and triethylamine (4.5 ml, 32.4 mmol) in dichloromethane (30 mL) was stirred at room temperature overnight. Solvent was evaporated and the residue was purified by Flash™ chromatography (BIOTAGE, Inc) (9.3:0.7 dichloromethane/methanol, v/v) to provide 3a (1.3 g, 42%) as a yellow oil. ¹H NMR (300 MHz, CD₃OD) δ 1.30 (m, 5H), 1.66 (m, 2H), 1.87 (d, 2H), 2.37 (m, 2H), 2.58 (m, 2H), 3.04 (m, 2H), 3.39 (m, 2H), 3.65 (s, 3H), 3.80 (m, 2H), 4.55 (s, 2H), 7.37 (m, 5H). m/z (ESI): 320 [C₁₉H₂₉NO₃+H]⁺.

### 4-[1-(2-Benzyloxyethyl)piperidin-4-yl]butyramide (4a)

Compound 3a (1.30 g, 4.0 mmol) was dissolved in 7 N NH₃ in methanol (25 mL) in a sealed tube. The resulting solution was stirred at 50° C. for 3 days. After that the solvent was removed under vacuum and the residue was purified by Flash™ chromatography (BIOTAGE, Inc) (9.5:0.45:0.05 dichloromethane/methanol/concentrated ammonium hydroxide, v/v) to provide 4a (0.93 g, 78%) as a white solid. ¹H NMR (300 MHz, CD₃OD) δ 1.27 (m, 5H), 1.65 (m, 4H), 2.11 (m, 4H), 2.65 (m, 2H), 2.96 (d, 2H), 3.62 (m, 2H), 4.51 (s, 2H), 7.37 (m, 5H). m/z (ESI): 305 [C₁₈H₂₈N₂O₂+H]⁺.

### 4-[1-(2-Benzyloxyethyl)piperidin-4-yl]butylamine (5a)

To a solution of BH₃.THF (2.2 mL, 2.2 mmol) cooled to 0° C. was added compound 4a (100 mg, 0.3 mmol). The resulting mixture was stirred for 30 min, then warmed to room temperature and stirred overnight. The reaction was quenched with water, and extracted with Et₂O. The organic solution was dried over Na₂SO₄ and concentrated under vacuum to provide 5a (85.2 mg, 89%) which was used directly without further purification. ¹H NMR (500 MHz, CD₃OD) δ 1.39 (m, 2H), 1.45 (m, 4H), 1.62 (m, 1H), 1.71 (m, 2H), 1.95 (m, 2H), 2.87 (m, 2H), 2.97 (m, 2H), 3.25 (m, 2H), 3.45 (d, 2H), 3.82 (m, 2H), 4.61 (s, 2H), 7.39 (m, 5H). m/z (ESI): 291 [C₁₈H₃₀N₂O+H]⁺.

### 2-[4-(4-Aminobutyl)piperidin-1-yl]ethanol (6a)

A suspension of 5a (0.3 g, 1.03 mmol) and catalyst (10% palladium on carbon, 0.8 g, 50% wet) in methanol (25 mL) was placed in a Parr shaker bottle. The system was vacuumed and flushed with nitrogen. The procedure was repeated three times. The mixture was then shaken at room temperature overnight under 40 psi hydrogen atmosphere. The system was then vacuumed again and flushed with nitrogen. The procedure was repeated three times. The catalyst was filtered under vacuum and washed with methanol (2×10 mL). The filtrate and washings were combined and concentrated under reduced pressure to provide 6a (186 mg, 90%). The crude product was used directly without purification. m/z (ESI): 201 [C₁₁H₂₄N₂O+H]⁺.

### N-(3,5-Diamino-6-chloropyrazine-2-carbonyl)-N′{4-[1-(2-hydroxyethyl)piperidin-4-yl]butyl}guanidine dihydrochloride (7a, PSA 25193)

1-(3,5-Diamino-6-chloropyrazine-2-carbonyl)-2-methylisothiourea hydriodide (290 mg, 0.73 mmol) was added to a solution of compound 6a (130 mg, 0.65 mmol) and DIPEA (0.34 mL, 1.95 mmol) in ethanol (5 mL). The reaction mixture was stirred at 65° C. for 5 h. Solvent was removed under reduced pressure and the residue was purified by semi-preparative HPLC (water/acetonitrile/0.1% TFA). The purified product was dissolved in 5% HCl aqueous solution and stirred at room temperature for 30 min. The mixture was then concentrated and further dried under high vacuum to provide 7a (15 mg, 6%) as a light yellow solid. ¹H NMR (500 MHz, CD₃OD) δ 1.50 (m, 9H), 2.01 (d, 2H), 3.05 (m, 2H), 3.20 (m, 2H), 3.61 (m, 2H), 3.89 (s, 2H). m/z (ESI): 413 [C₁₇H₂₉ClN₈O₂+H]⁺. mp 168-170° C.

### Example 2

### Synthesis of N-(3,5-diamino-6-chloropyrazine-2-carbonyl)-N′-{4-[1-(3-hydroxypropyl)piperidin-4-yl]butyl}guanidine dihydrochloride (PSA 25310)

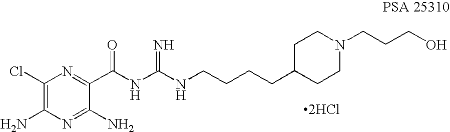

### 4-[1-(3-Benzyloxypropyl)piperidin-4-yl]butyric acid methyl ester (3b)

Following the same procedure described for the preparation of compound 3a, the compound 3b was synthesized in 40% yield from compound 2 as a yellow oil.

¹H NMR (300 MHz, CD₃OD) δ 1.21 (m, 4H), 1.42 (m, 1H), 1.49 (m, 2H), 1.83 (d, 2H), 1.93 (m, 2H), 2.31 (m, 2H), 2.69 (m, 2H), 2.99 (m, 2H), 3.35 (m, 2H), 3.60 (m, 5H), 4.50 (m, 2H), 7.28 (m, 5H). m/z (ESI): 334 [C₂₀H₃₁NO₃+H]⁺.

### 4-[1-(3-Benzyloxypropyl)piperidin-4-yl]butyramide (4b)

Following the same procedure described for the preparation of compound 4a, compound 4b was synthesized in 69% yield from compound 3b as a yellow solid.

¹H NMR (500 MHz, DMSO-d₆) δ 1.25 (m, 5H), 1.49 (m, 2H), 1.68 (m, 2H), 1.85 (m, 2H), 2.01 (m, 2H), 2.40 (m, 2H), 2.75 (m, 2H), 3.13 (m, 3H), 3.45 (m, 3H), 4.47 (m, 2H), 7.37 (m, 5H). m/z (ESI): 319 [C₁₉H₃₀N₂O₂+H]⁺.

### 4-[1-(3-Benzyloxypropyl)piperidin-4-yl]butylamine (5b)

Following the same procedure described for the preparation of compound 5a, compound 5b was synthesized in 70% yield from compound 4b as a light yellow solid. ¹H NMR (500 MHz, CDCl₃) δ 1.16 (m, 5H), 1.29 (m, 2H), 1.43 (m, 2H), 1.61 (m, 3H), 1.85 (m, 5H), 2.60 (m, 3H), 2.70 (m, 1H), 2.95 (m, 2H), 3.50 (m, 2H), 4.51 (s, 2H), 7.39 (m, 5H). m/z (ESI): 305 [C₁₉H₃₂N₂O+H]⁺.

### 3-[4-(4-Aminobutyl)piperidin-1-yl]propan-1-ol (6b)

Following the same procedure described for the preparation of compound 6a, compound 6b was synthesized in 90% yield from compound 5b as a light yellow solid. ¹H NMR (500 MHz, CDCl₃) δ 1.20 (m, 7H), 1.41 (m, 2H), 1.65 (m, 5H), 1.89 (m, 2H), 2.60 (m, 4H), 3.00 (m, 4H), 3.79 (m, 2H). m/z (ESI): 215 [C₁₂H₂₆N₂O+H]⁺.

### N-(3,5-Diamino-6-chloropyrazine-2-carbonyl)-N′-{4-[1-(3-hydroxypropyl)piperidin-4-yl]butyl}guanidine dihydrochloride (7b, PSA 25310)

Following the same procedure described for the preparation of compound 7a, compound 7b was synthesized in 40% yield from compound 6b as a yellow solid.

¹H NMR (500 MHz, DMSO-d₆) δ 1.25 (m, 5H), 1.52 (m, 5H), 1.85 (m, 4H), 2.85 (m, 2H), 3.00 (m, 2H), 3.15 (m, 1H), 3.31 (m, 2H), 3.45 (m, 4H), 7.41 (m, 3H), 8.90 (m, 2H), 9.40 (m, 1H). m/z (ESI): 427 [C₁₈H₃₁ClN₈O₂+H]⁺. mp 165-167° C.

### Example 3

### Synthesis of N-{4-[1-(2-aminoethyl)piperidin-4-yl]butyl}-N-(3,5-diamino-6-chloro-pyrazine-2-carbonyl)guanidine trihydrochloride (PSA 25455)

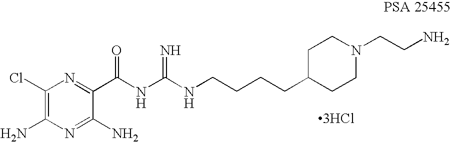

### 4-[1-(2-tert-Butoxycarbonylaminoethyl)piperidin-4-yl]butyric acid methyl ester (3c)

Following the same procedure described for the preparation of compound 3a, compound 3c was synthesized from compound 2 as an off white solid. ¹H NMR (300 MHz, CD₃OD) δ 1.18-1.35 (m, 7H), 1.41 (m, 9H), 1.59-1.84 (m, 5H), 2.29-2.37 (m, 2H), 2.41-2.52 (m, 2H), 2.86-3.02 (m, 2H), 3.13-3.24 (m, 2H), 3.67 (s, 3H). m/z (ESI): 329 [C₁₇H₃₂N₂O₄+H]⁺.

### {2-[4-(3-Carbamoylpropyl)piperidin-1-yl]ethyl}carbamic acid tert-butyl ester (4c)

Following the same procedure described for the preparation of compound 4a, compound 4c was synthesized from compound 3c as an off-white solid. ¹H NMR (300 MHz, CD₃OD) δ 1.18-1.35 (m, 7H), 1.41 (m, 9H), 1.59-1.84 (m, 5H), 2.29-2.37 (m, 2H), 2.41-2.52 (m, 2H), 2.86-3.02 (m, 2H), 3.13-3.24 (m, 2H). m/z (ESI): 314 [C₁₆H₃₁N₃O₃+H]⁺.

### {2-[4-(4-Aminobutyl)piperidin-1-yl]ethyl}carbamic acid tert-butyl ester (5c)

A solution of compound 4c (250 mg, 0.80 mmol) in dichloromethane (10 mL) was cooled to 0° C., then DIBA1-H (7.4 mL, 7.4 mmol of 1M in toluene) was added dropwise into the solution over 45 min. The mixture was stirred for 1 hour, then warmed to room temperature and stirred for 14 h. The reaction was quenched with potassium sodium tartrate aqueous solution. The mixture was extracted with dichloromethane (3×10 mL). The combined extracts were washed with water and brine, dried over sodium sulfate and concentrated under vacuum to afford an oil. Purification by column chromatography (silica; 90:10, v/v, dichloromethane/methanol followed by 89:10:1 dichloromethane/methanol/ammonium hydroxide) produced the desired product 5c (54 mg, 23% un-optimized yield) as a clear colorless oil. ¹H NMR (500 MHz, CDCl₃) δ 1.21-1.26 (m, 8H), 1.42-1.46 (m, 11H), 1.641.67 (m, 3H), 1.91-1.99 (m, 2H), 2.41-2.48 (m, 2H), 2.67-2.70 (m, 2H), 2.83-2.86 (m, 2H), 3.20-3.22 (m, 2H), 5.00 (br s, 1H). m/z (ESI): 300 [C₁₆H₃₃N₃O₂+H]⁺.

### [3-(4-{4-[N′-(3,5-Diamino-6-chloropyrazine-2-carbonyl)guanidino]butyl}piperidin-1-yl)ethyl]carbamic acid tert-butyl ester (7c)

Following the same procedure described for the preparation of compound 7a, compound 7c was synthesized in 54% yield from compound 5c as a yellow solid (Scheme 2). ¹H NMR (500 MHz, CDCl₃) δ 1.21-1.26 (m, 8H), 1.41-1.46 (m, 11H), 1.64-1.67 (m, 7H), 1.91-1.99 (m, 2H), 2.41-2.48 (m, 2H), 2.83-2.86 (m, 2H), 3.20-3.22 (m, 2H), 5.00 (br s, 2H). m/z (ESI): 512 [C₂₂H₃₈ClN₉O₃+H]⁺.

### N-{4-[1-(2-Aminoethyl)piperidin-4-yl]butyl}-N′-(3,5-diamino-6-chloropyrazine-2-carbonyl)guanidine trihydrochloride (8c, PSA 25455)

A solution of compound 7c (37 mg, 0.0723 mmol) dissolved in methanol (2 mL) was cooled to 0° C. (Scheme 2). To the stirring solution was added dropwise 1 N HCl in diethyl ether (1 mL). The resulting mixture was stirred for 2 h, then the solvent was removed under vacuum and the residue was dried under high vacuum to provide 8c (36 mg, quant) as a yellow solid: mp>200° C. ¹H NMR (500 MHz, DMSO-d₆) δ 1.24-1.92 (m, 12H), 2.82-3.02 (m, 2H), 3.51-3.72 (m, 4H), 7.45-7.58 (m, 2H), 8.42 (br s, 3H), 8.75-9.09 (m, 2H), 9.29 (br s, 1H), 10.55 (br s, 1H), 10.75 (m, 1H). m/z (APCI): 412 [C₁₇H₃₀ClN₉O+H]⁺.

### Example 4

### Synthesis of N-{4-[1-(3-aminopropyl)piperidin-4-yl]butyl}-N′-(3,5-diamino-6-chloropyrazine-2-carbonyl)guanidine trihydrochloride (PSA 25510)

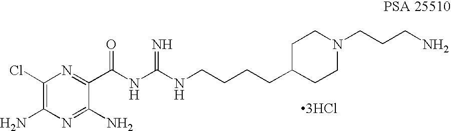

### 4-[1-(3-tert-Butoxycarbonylaminopropyl)piperidin-4-yl]butyric acid methyl ester (3d)

Following the same procedure described for the preparation of compound 3a, compound 3d was synthesized in 64% yield from compound 2 as a yellow solid. ¹H NMR (500 MHz, CDCl₃) δ 1.30 (m, 3H), 1.41 (m, 12H), 1.65 (m, 3H), 1.78 (m, 2H), 1.95 (m, 2H), 2.25 (m, 3H), 2.75 (m, 1H), 3.17 (m, 4H), 3.67 (m, 3H), 4.98 (s, 1H). m/z (ESI): 343 [C₁₈H₃₄N₂O₄+H]⁺.

### {3-[4-(3-Carbamoylpropyl)piperidin-1-yl]propyl}carbamic acid tert-butyl ester (4d)

Following the same procedure described for the preparation of compound 4a, compound 4d was synthesized in 66% yield from compound 3d as a yellow solid.

¹H NMR (500 MHz, CDCl₃) δ 1.22 (m, 7H), 1.45 (s, 9H), 1.65 (m, 6H), 1.87 (m, 2H), 2.19 (m, 2H), 2.39 (m, 2H), 2.90 (d, 2H), 5.40 (s, 2H), 5.62 (s, 1H). m/z (ESI): 328 [C₁₇H₃₃N₃O₃+H]⁺.

### {3-[4-(4-Aminobutyl)piperidin-1-yl]propyl}carbamic acid tert-butyl ester (5d)

Following the same procedure described for the preparation of compound 5c, compound 5d was synthesized in 82% yield from compound 4d as an off-white solid. ¹H NMR (500 MHz, CDCl₃) δ 1.20 (m, 5H), 1.35 (m, 3H), 1.46 (m, 12H), 1.65 (m, 2H), 1.84 (m, 2H), 2.46 (m, 2H), 2.68 (m, 1H), 2.87 (d, 2H), 3.18 (d, 2H), 3.45 (s, 1H), 5.65 (s, 2H), 7.49 (m, 1H). m/z (ESI): 314 [C₁₇H₃₅N₃O₂+H]³⁰ .

### [3-(4-{4-[N′-(3,5-diamino-6-chloropyrazine-2-carbonyl)guanidino]butyl}piperidin-1-yl)propyl]carbamic acid tert-butyl ester (7d, PSA 25452)

Following the same procedure described for the preparation of compound 7c, compound 7d was synthesized from compound 5d as a yellow solid (Scheme 2). ¹H NMR (500 MHz, DMSO-d₆) δ 1.12 (m, 6H), 1.31 (m, 11H), 1.47 (m, 4H), 1.60 (d, 2H), 1.77 (m, 2H), 2.20 (m, 2H), 2.79 (d, 2H), 2.91 (m, 2H), 3.10 (m, 3H), 6.55 (m, 3H), 6.79 (s, 2H), 9.05 (s, 1H). m/z (APCI): 527 [C₂₃H₄₀ClN₉O₃+H]³⁰ . mp 98-102° C.

### N-{4-[1-(3-Aminopropyl)piperidin-4-yl]butyl}-N′-(3,5-diamino-6-chloropyrazine-2-carbonyl)guanidine trihydrochloride (8d, PSA 25510)

Following the same procedure described for the preparation of compound 8c, compound 8d was synthesized in 91% yield from compound 7d as a yellow solid (Scheme 2). ¹H NMR (500 MHz, DMSO-d₆) δ 1.30 (m, 4H), 1.55 (m, 5H), 1.85 (d, 2H), 2.07 (m, 2H), 2.85 (m, 3H), 3.12 (m, 2H), 3.31 (m, 2H), 3.44 (m, 2H), 7.45 (m, 2H), 8.19 (s, 3H), 8.90 (d, 2H), 9.35 (s, 1H), 10.55 (s, 1H), 10.75 (s, 1H). m/z (ESI): 426 [C₁₈H₃₂ClN₉O+H]⁺. mp 105-108° C.

### Example 5

### N-(3,5-Diamino-6-chloropyrazine-2-carbonyl)-N′-{4-[1-(2,3-dihydroxypropyl)-piperidin-4-yl]butyl}guanidine (PSA 25456)

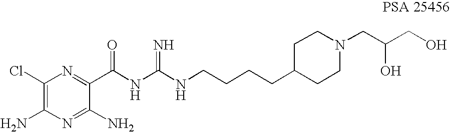

### 4-[1-(2,3-Dihydroxypropyl)piperidin-4-yl]butyramide (10)

Following the same procedure as described for the preparation of compound 4, compound 10 (263 mg, 71% yield, Scheme 3) was prepared from compound 9 as a clear orange oil. ¹H NMR (500 MHz, CDCl₃) δ 1.21-1.29 (m, 6H), 1.65-1.70 (m, 6H), 2.92-2.98 (m, 1H), 2.19-2.34 (m, 3H), 2.51-2.53 (m, 1H), 2.78-2.82 (m, 1H), 2.96-3.02 (m, 1H), 3.45-3.75 (m, 3H), 5.28 (m, 2H). m/z (ESI): 245 [C₁₂H₂₄N₂O₃+H]⁺.

### 3-[4-(4-Aminobutyl)piperidin-1-yl]propane-1,2-diol (11)

Compound 10 (263 mg, 1.07 mmol) was dissolved in tetrahydrofuran (12 mL) under a nitrogen atmosphere. Lithium aluminum hydride (3.7 mL of a 1 M solution in THF) was added dropwise over 20 min. The reaction was refluxed for 8 h, and then cooled to room temperature. It was quenched by successively adding water (1 mL, dropwise), 20% sodium hydroxide solution (1 mL), and then 25% ammonium hydroxide solution (2 mL). The resulting mixture was stirred for 30 min and then filtered through diatomaceous earth. The filtrate was dried over sodium sulfate and concentrated under vacuum to give the amine 11 (183 mg, 74% yield) as a red oil which was carried on without further purification: ¹H NMR (500 MHz, CDCl₃) δ 1.21-1.68 (m, 12H), 1.88-1.95 (m, 3H), 2.20-2.33 (m, 4H), 2.49-2.53 (m, 1H), 2.66-2.70 (m, 1H), 2.78-2.82 (m, 1H), 2.96-3.02 (m, 1H), 3.48-3.94 (m, 3H). m/z (ESI): 231 [C₁₂H₂₆N₂O₂+H]⁺.

### N-(3,5-Diamino-6-chloropyrazine-2-carbonyl)-N′-{4-[1-(2,3-dihydroxypropyl)-piperidin-4-yl]butyl}guanidine (12, PSA 25456)

Following the same procedure described for the preparation of compound 7a, compound 12 was synthesized in 28% yield from compound 11 as a yellow solid. mp 188-191° C. ¹H NMR (500 MHz, DMSO-d₆) δ 1.09-1.32 (m, 8H), 1.45-1.61 (m, 4H), 1.90 (br s, 2H), 2.20-2.30 (m, 2H), 3.75-3.92 (m, 2H), 3.11 (br s, 2H), 3.57 (br s, 1H), 4.31 (br s, 1H), 4.56-4.57 (m, 1H), 6.60 (br s, 3H), 9.06 (br s, 1H). m/z (APCI): 443 [C₁₈H₃₁ClN₈O₃+H]⁺.

### Example 6

### Synthesis of N-(3,5-Diamino-6-chloropyrazine-2-carbonyl)-N′-{4-[1-(3-guanidino-propyl)piperidin-4-yl]butyl}guanidine trihydrochloride (PSA 25795)

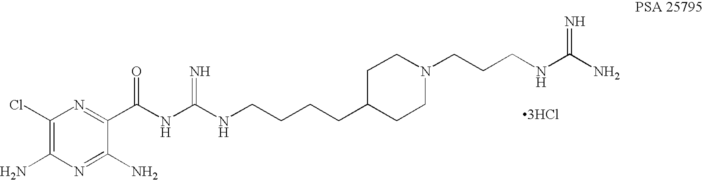

### N-(3,5-Diamino-6-chloropyrazine-2-carbonyl)-N′-{4-[1-(3-[N″,N′″-bis-tert-butoxycarbonyl]guanidinopropyl)piperidin-4-yl]butyl}guanidine (13, PSA 25569)

The Goodman's reagent, (tert-Butoxycarbonylamino-trifluoromethanesulfonylimino-methyl)carbamic acid tert-butyl ester, (368 mg, 0.94 mmol) was added to a solution of compound 8d (360 mg, 0.67 mmol) and DIPEA (0.47 mL, 2.69 mmol) in methanol (20 mL). The reaction mixture was stirred at room temperature overnight. Solvent was removed under reduced pressure and the residue was purified by flash silica gel chromatography (9:0.9:0.1 dichloromethane/methanol/concentrated ammonium hydroxide, v/v) to provide 13 (327 mg, 73%) as a yellow solid. mp 122-125° C. ¹H NMR (500 MHz, DMSO-d₆) δ 1.25 (m, 9H), 1.40 (m, 21H), 1.59 (m, 4H), 1.75 (m, 2H), 2.25 (m, 2H), 2.82 (m, 2H), 3.11 (m, 2H), 6.60 (m, 3H), 8.55 (s, 2H), 9.05 (s, 1H), 11.55 (s, 2H). m/z (ESI) 668 [C₂₉H₅₀ClN₁₁O₅+H]⁺.

### N-(3,5-Diamino-6-chloropyrazine-2-carbonyl)-N′-{4-[1-(3-guanidinopropyl)piperidin-4-yl]butyl}guanidine trihydrochloride (14, PSA 25795)

To a solution of compound 13 (250 mg, 0.37 mmol) in methanol (5 mL) cooled at 0° C. was added dropwise 12 N HCl (2.5 mL). It was stirred first at 0° C. for 0.5 h, then allowed to warm up to room temperature. The stirring was continued for an additional 3 h. Complete removal of solvent under vacuum provided 14 (215 mg, 94%) as a yellow solid. mp 176-178° C. ¹H NMR (500 MHz, DMSO-d₆) δ 1.29 (m, 2H), 1.30 (m, 3H), 1.54 (m, 6H), 1.82 (m, 2H), 1.93 (m, 2H), 2.84 (m, 2H), 3.02 (m, 2H), 3.15 (s, 1H), 3.24 (m, 5H), 7.17 (m, 3H), 7.99 (s, 1H), 8.90 (d, 2H), 9.30 (s, 1H), 10.54 (s, 1H), 10.62 (s, 1H). m/z (ESI) 468 [C₁₉H₃₄ClN₁₁O+H]⁺.

### Example 7

Sodium Channel Blocking Activity of Selected Cyclic Pyrazinoylguanidines

| | | | Fold Amiloride** | |
|---|---|---|---|---|
| PSA | EC₅₀(nM) | | (PSA 4022 = 100) | |
| 25310 | 169 ± 47 | (n = 8) | 5 ± 2 | (n = 8) |
| 25193 | 99 ± 14 | (n = 6) | 8 ± 3 | (n = 6) |
| 25452 | 60 ± 6 | (n = 6) | 8 ± 1 | (n = 6) |
| 25455 | 104 ± 32 | (n = 7) | 5 ± 1 | (n = 7) |
| 25456 | 106 ± 34 | (n = 7) | 6 ± 2 | (n = 7) |
| 25510 | 61 ± 23 | (n = 7) | 3 ± 9 | (n = 7) |
| 25569 | 16 ± 3 | (n = 4) | 41 ± 9 | (n = 9) |
| 25795 | 37 ± 5 | (n = 4) | 18 ± 7 | (n = 4) |

**Relative potency for PSA 4022 = 100 using EC₅₀ from PSA 4022 in same run

While the invention has been described with reference to preferred aspects or embodiments, it is to be understood that variations and modifications may be resorted to as will be apparent to those skilled in the art. Such variations and modifications are to be considered within the purview and the scope of the claims appended hereto.

1. A prophylactic treatment method comprising:
administering a prophylactically effective amount of a sodium channel blocker according to Formula I: 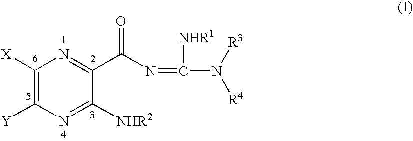
wherein
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—(CH₂OR⁸, —(CH₂)ₙ—CO₂R⁷, or 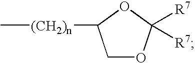
R³ and R⁴ are each, independently, hydrogen, a group represented by formula (A), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R³ and R⁴ is a group represented by formula (A): 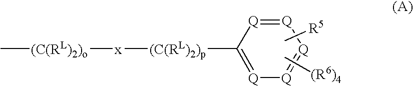
wherein
each R^{L} is, independently, —R⁷, —(CH₂)ₙ—OR⁸, —O—(CH₂)ₘ—OR⁸, —(CH₂)ₙ—NR⁷R¹⁰, —O(CH₂)ₘNR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷—O—(CH₂)ₘ—CO₂R⁷, —OSO₃H, —O-glucuronide, —O-glucose, 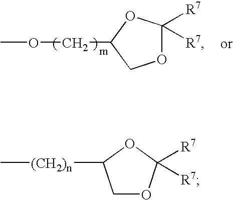
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
wherein each R⁵ is, independently,
Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ—(Z)_{g}—CAP, Link —(CH₂)ₙ(Z)_{g}—(CH₂)ₘ—CAP, Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}—CAP, Link —(CH₂)ₙNR¹³—(CH₂)ₘ(CHOR⁸)ₙCH₂NR¹³-(Z)_{g}—CAP, Link —(CH₂)ₘ-(Z)_{g}-(CH₂)ₘ—CAP, Link NH—C(═O)—NH—(CH₂)ₘ—CAP, Link —(CH₂)ₘ—C(═O)NR¹³—(CH₂)ₘ—C(═O)NR¹⁰OR¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)ₘ-Het-(CH₂)ₘ—CAP;
wherein Link is, independently,
—O—, (CH₂)ₙ—, —O(CH₂)ₘ—, —NR¹³—C(═O)—NR¹³, —NR¹³—C(═O)—(CH₂)ₘ—, —C(═O)NR¹³—(CH₂)ₘ, —(CH₂)ₙ-Z_{g}-(CH₂)_{n,}—S—, —SO—, —SO₂—, SO₂NR⁷—, SO₂NR¹⁰—, -Het-;
wherein each CAP is, independently,
thiazolidinedione, oxazolidinedione, heteroaryl-C(═O)N R¹³R¹³ , heteroaryl-CAP, —CN, —O—C(═S)NR¹³R¹³, -Z_{g}R¹³, —CR¹⁰(Z_{g}R¹³)(Z_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, cyclic sugars and oligosaccharides, including cyclic amino sugars and oligosaccharides, 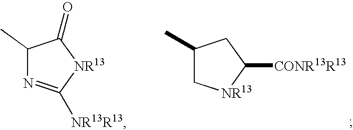
wherein Ar is, independently, phenyl; substituted phenyl, wherein said substituent is 1-3 groups selected, independently, from OH, OCH₃, NR¹³R¹³, Cl, F, CH₃; or heteroaryl, tinazine, furyl, furfuryl-, thienyl, tetrazole, thiazolidinedione, or imidazoyl ( 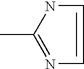
);
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
each R⁶ is, independently, —R⁷, —OR⁷, —OR¹¹, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘCO₂R⁷, —OSO₃H, —O-glucuronide, —O-glucose, 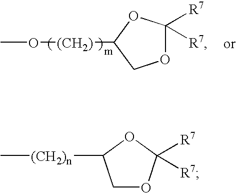
where when two R⁶ are —OR¹¹ and are located adjacent to each other on a phenyl ring, the alkyl moieties of the two R6 may be bonded together to form a methylenedioxy group;
with the proviso that when at least two —CH₂OR⁸ are located adjacent to each other, the R⁸ groups may be joined to form a cyclic mono- or di-substituted 1,3-dioxane or 1,3-dioxolane,
each R⁷is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 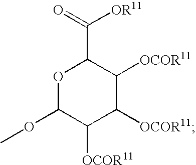
each R⁹ is, independently, —CO₂R¹³, —CON(R¹³)₂, —SO₂CH₂R¹³, or —C(═O)R¹³;
each R¹⁰ is, independently, —H, —SO₂CH₃, —CO₂R¹³, —C(═O)NR¹³R¹³, —C(═O)R¹³, or —(CH₂)ₘ—(CHOH)ₙ—CH₂OH;
each Z is, independently, CHOH, C(═O), —(CH₂)ₙ—,CHNR¹³R¹³, C═NR¹³, or NR¹³;
each R¹¹ is, independently, lower alkyl;
each R¹² is independently, —SO₂CH₃, —CO₂R¹³, —C(═O)NR¹³R¹³, —C(═O)R¹³, or —CH₂—(CHOH)ₙ—CH₂OH;
each R¹³ is, independently, hydrogen, R⁷, R¹⁰, CH₂)ₘ—NR¹³R¹³, 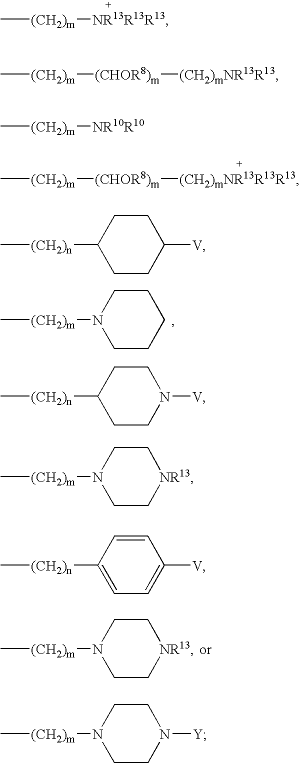
with the proviso that NR¹³R¹³ can be joined on itself to form a ring comprising one of the following: 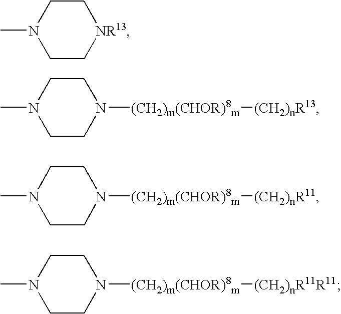
each Het is independently, —NR¹³, —S—, —SO—, or —SO₂—; —O—, —SO₂NR¹³, —NHSO₂—, —NR¹³CO—, —CONR¹³;
each g is, independently, an integer from 1 to 6;
each m is, independently, an integer from 1 to 7;
each n is, independently, an integer from 0 to 7;
each Q is, independently, C—R⁵, C—R⁶, or a nitrogen atom, wherein at most three Q in a ring are nitrogen atoms;
each V is, independently, —(CH₂)ₘ—NR⁷R¹⁰, —(CH₂)ₘ—NR⁷R⁷, —(CH₂)ₘ— 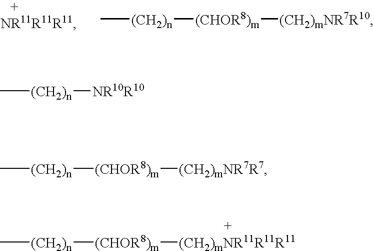
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3-with respect to each other the R⁸ groups may be joined to form a cyclic mono- or di-substituted 1,3-dioxane or 1,3-dioxolane; or a pharmaceutically acceptable salt thereof, to an individual in need of prophylactic treatment against infection or disease from one or more airborne pathogens.

2. The prophylactic treatment method of claim 1 wherein the pathogen is Bacillus anthracis.

3. The prophylactic treatment method of claim 1 wherein the pathogen is Variola major.

4. The prophylactic treatment method of claim 1 wherein the pathogen is Yersinia pestis.

5. The prophylactic treatment method of claim 1 wherein the pathogen is Francisella tularensis.

6. The prophylactic treatment method of claim 1 wherein the pathogen is a gram negative bacteria.

7. The prophylactic treatment method of claim 6 wherein the gram negative bacteria is selected from the group consisting of Brucella species, Burkholderia pseudomallei, Burkholderia mallei, Coxiella burnetii and Rickettsia.

8. The prophylactic treatment method of claim 1 wherein the pathogen is an alphavirus, a flavivirus or a bunyavirus.

9. The prophylactic treatment method of claim 1 wherein the pathogen is ricin toxin from Ricinus communis, epsilon toxin of Clostridium perfringens or Staphylococcal enterotoxin B.

10. The prophylactic treatment method of claim 1 wherein the pathogen is Mycobacterium tuberculosis bacteria.

11. The prophylactic treatment method of claim 1 wherein the pathogen is an influenza virus, rhinovirus, adenovirus or respiratory syncytial virus.

12. The prophylactic treatment method of claim 1 wherein the pathogen is coronavirus.

13. The prophylactic treatment method of claim 1 wherein the sodium channel blocker or pharmaceutically acceptable salt thereof is administered in an aerosol suspension of respirable particles which the individual inhales.

14. The prophylactic treatment method of claim 1 wherein the sodium channel blocker or pharmaceutically acceptable salt thereof is administered for reducing the risk of infection from an airborne pathogen which can cause a disease in a human to the lungs of the human who may be at risk of infection from the airborne pathogen but is asymptomatic for the disease, wherein the effective amount of sodium channel blocker or a pharmaceutically acceptable salt is sufficient to reduce the risk of infection in the human.

15. The prophylactic treatment method of claim 1 wherein the sodium channel blocker or pharmaceutically acceptable salt thereof is administered post-exposure to the one or more airborne pathogens.

16. The prophylactic treatment method of claim 1 wherein the sodium channel blocker is selected from the group consisting of:

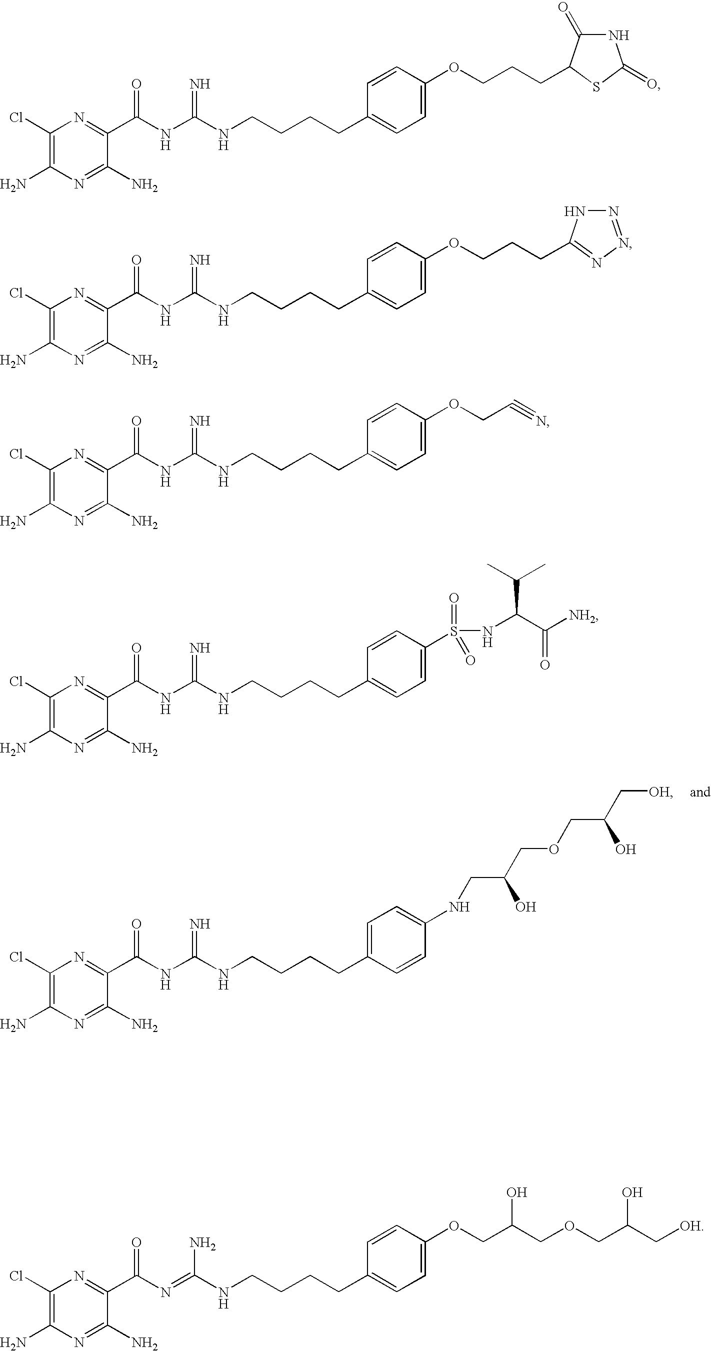

17. The prophylactic treatment method of claim 1 wherein the sodium channel blocker is selected from the group consisting of:

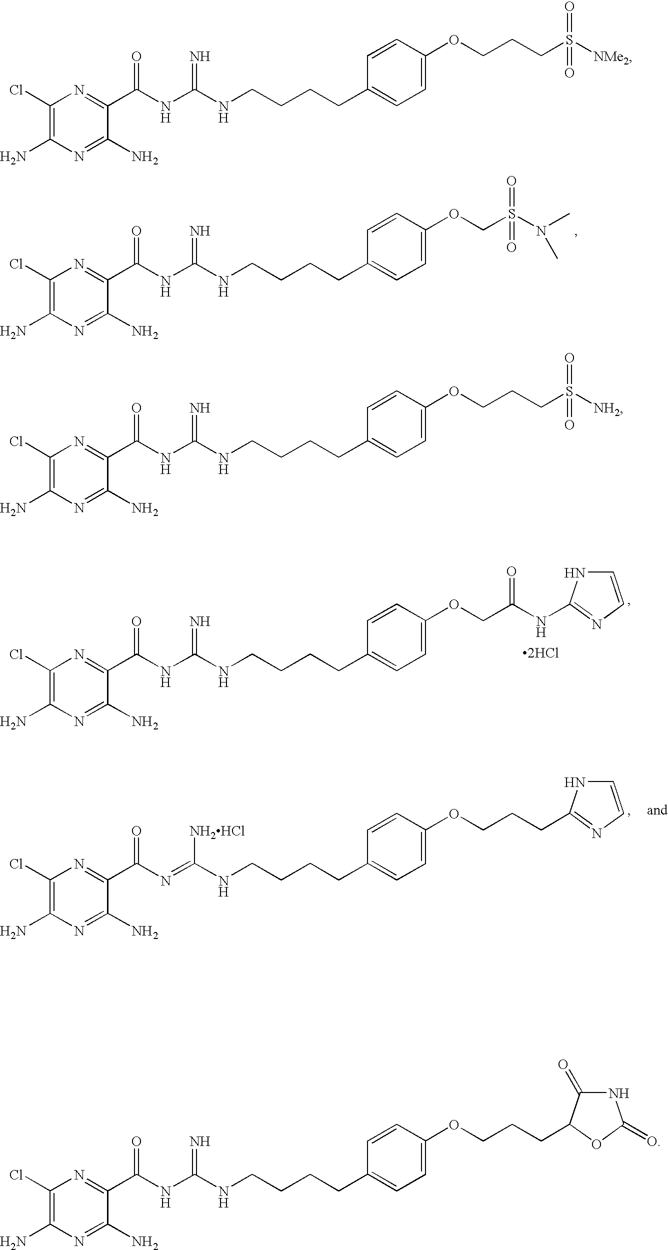

18. The prophylactic treatment method of claim 1 wherein the sodium channel blocker is selected from the group consisting of:

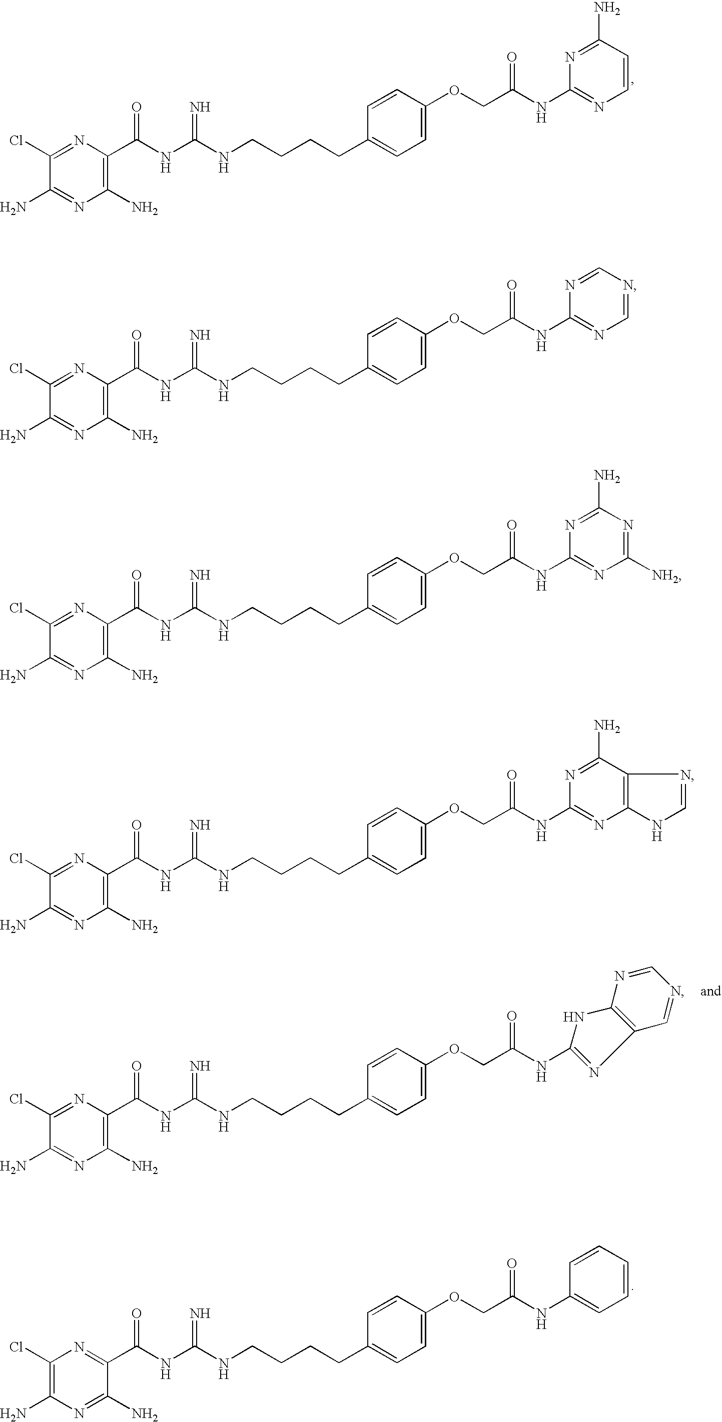

19. A prophylactic treatment method comprising: administering a prophylactically effective amount of a sodium channel blocker according to Formula II: 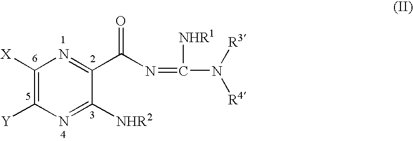
where
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 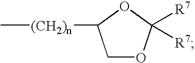
R^{3′} and R^{4′} are each, independently, hydrogen, a group represented by formula (A′), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R^{3′} and R^{4′} is a group represented by formula (A′):
—(C(R^{L})₂)_{O}-x-(C(R^{L})₂)_{P}—CR^{5′}R^{6′}R^{6′} (A′)
where
each R^{L} is, independently, —R⁷, —(CH₂)ₙ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 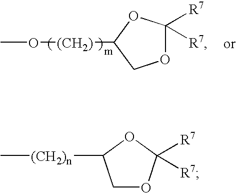
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
each R^{5′} is, independently, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙCH₂OR⁸, —O—(CH₂)ₘ(CHOR⁸)(CHOR⁸)ₙ—CH₂OR⁸, —(CH₂CH₂O)ₘR⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH²OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—O₂R⁷, —OSO₃H, —O-glucuronide, —O-glucose, 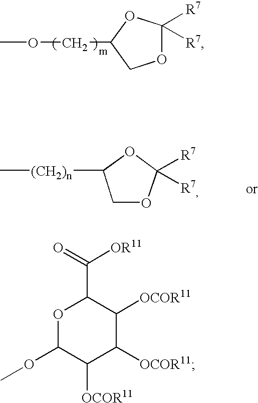
each R^{5′} is also, independently, —(CH₂)ₙ—NR¹²R¹², —O—(CH₂)ₘ—NR¹²R¹², —O—(CH₂)ₙ—NR¹²R¹², —O—(CH₂)ₘ-(Z)_{g}R¹², —(CH₂)ₙNR¹¹R¹¹, —O—(CH₂)ₘNR¹¹R¹¹, —(CH₂)ₙ—N^{⊕}—(R¹¹)₃, —O—(CH₂)ₘ—N^{⊕}—(R¹¹)₃, —(CH₂)ₙ-(Z)_{g}—(CH₂)ₘ—NR¹⁰R¹⁰, —O—(CH₂)ₘ-(Z)_{g}-(CH₂)ₘ—NR¹⁰R¹⁰, —(CH₂CH₂O)ₘ—CH₂CH₂NR¹²R¹², —O—(CH₂CH₂O)ₘ—CH₂CH₂NR¹²R¹², —(CH₂)ₙ—(C═O)NR¹²R¹², —O—(CH₂)ₘ—(C═O)NR¹²R¹², —O—(CH₂)ₘ—(CHOR⁸)ₘCH₂NR¹⁰—(Z)_{g}-R¹⁰, —(CH₂)ₙ—(CHOR⁸)ₘCH₂—NR¹⁰-(Z)_{g}-R¹⁰, —(CH₂)ₙNR¹⁰—O(CH₂)ₘ(CHOR⁸)ₙCH₂NR¹⁰-(Z)_{g}-R¹⁰, —O(CH₂)ₘ—NR¹⁰—(CH₂)ₘ—(CHOR⁸)ₙCH₂NR¹⁰-(Z)_{g}-R¹⁰, -(Het)-(CH₂)ₘ—OR⁸, -(Het)-(CH₂)ₘ—NR⁷R¹⁰, -(Het)-(CH₂)ₘ(CHOR⁸)(CHOR⁸)ₙ—CH₂OR⁸, -(Het)-(CH₂CH₂O)ₘ—R⁸, -(Het)-(CH₂CH₂O)ₘ—CH₂CH₂NR⁷R¹⁰, -(Het)-(CH₂)ₘ—C(═O)NR⁷R¹⁰, -(Het)-(CH₂)ₘ-(Z)_{g}-R⁷, -(Het)-(CH₂)ₙ—NR¹⁰—CH₂(CHOR⁸)(CHOR⁸)ₙ—CH₂OR⁸, -(Het)-(CH₂)ₘ—CO₂R⁷, -(Het)-(CH₂)ₘ—NR¹²R¹², -(Het)-(CH₂)ₙ—NR¹²R¹², -(Het)-(CH₂)ₘ-(Z)_{g}R¹², -(Het)-(CH₂)ₘNR¹¹R¹¹, -(Het)-(CH₂)ₘ—N^{⊕}—(R¹¹)₃, -(Het)-(CH₂)ₘ-(Z)_{g}-(CH₂)ₘ—NR¹⁰R¹⁰, -(Het)-(CH₂CH₂O)ₘ—CH₂CH₂NR¹²R¹², -(Het)-(CH₂)ₘ—(C═O)NR¹²R¹², -(Het)-(CH₂)ₘ—(CHOR⁸)ₘCH₂NR¹⁰-(Z)_{g}-R¹⁰, -(Het)-(CH₂)ₘ—NR¹⁰—(CH₂)ₘ—(CHOR⁸)ₙCH₂NR¹⁰-(Z)_{g}-R¹⁰,
wherein when two —CH₂OR⁸ groups are located 1,2- or 1,3-with respect to each other the R⁸ groups may be joined to form a cyclic mono- or di-substituted 1,3-dioxane or 1,3-dioxolane,
—(CH₂)ₙ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—O—(CH₂)ₘ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—(CH₂)ₙ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane, or
—O—(CH₂)ₘ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane;
wherein each R^{5′} is also, independently,
Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ—(Z)_{g}—CAP, Link —(CH₂)ₙ(Z)_{g}-(CH₂)ₘ—CAP, Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}-CAP, Link —(CH₂)ₙNR¹³—(CH₂)ₘ(CHOR⁸)ₙCH₂NR¹³-(Z)_{g}-CAP, Link —(CH₂)ₘ-(Z)_{g}-(CH₂)ₘ—CAP, Link NH—C(═O)—NH—(CH₂)ₘ—CAP, Link —(CH₂)ₘ—C(═O)NR¹³—(CH₂)ₘ—C(═O)NR¹⁰R¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)ₘ-Het-(CH₂)ₘ—CAP;
wherein Link is, independently,
—O—, (CH₂)ₙ—, —O(CH₂)ₘ—, —NR¹³—C(═O)—NR¹³, —NR¹³—C(═O)—(CH₂)ₘ—, —C(═O)NR¹³—(CH₂)ₘ, —(CH₂)ₙ-Z_{g}-(CH₂)ₙ, —S—, —SO—, —SO₂—, SO₂NR⁷—, SO₂NR¹⁰—, —Het-.
wherein each CAP is, independently,
thiazolidinedione, oxazolidinedione, heteroaryl-C(═O)N R¹³R¹³, heteroaryl-CAP, —CN, —O—C(═S)NR¹³R¹³, -Z_{g}R¹³, —CR¹⁰(Z_{g}R¹³)(Z_{g}R¹³), —C(═O)OAr, —C(═O)N R¹³Ar, imidazoline, tetrazole, tetrazole amide, —SO₂NHR¹³, —SO₂NH—C(R¹³R¹³)-(Z)_{g}-R¹³, cyclic sugars and oligosaccharides, including cyclic amino sugars and oligosaccharides, 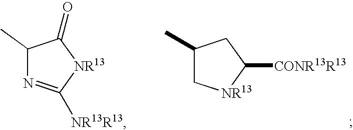
wherein Ar is, independently, phenyl; substituted phenyl, wherein said substituent is 1-3 groups selected, independently, from OH, OCH₃, NR¹³R¹³, Cl, F, CH₃; heteroaryl, tinazine, furyl, furfuryl-, thienyl, tetrazole, thiazolidinedione, or imidazoyl ( 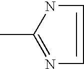
);
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
each R^{6′} is, independently, —R^{5′}, —R⁷, —OR⁸, —N(R⁷)₂, —(CH₂)ₘOR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙCH₂OR, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 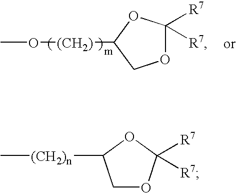
wherein when two —CH₂OR⁸ groups are located 1,2- or 1,3-with respect to each other the R⁸ groups may be joined to form a cyclic mono- or di-substituted 1,3-dioxane or 1,3-dioxolane;
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 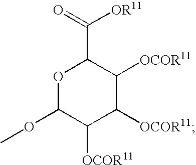
each R⁹ is, independently, —CO₂R¹³, —CON(R¹³)₂, —SO₂CH₂R¹³, or —C(═O)R¹³;
each R¹⁰ is, independently, —H, —SO₂CH₃, —CO₂R¹³, —C(═O)NR¹³R¹³, —C(═O)R³, or —(CH₂)ₘ—(CHOH)ₙ—CH₂OH;
each Z is, independently, CHOH, C(═O), —(CH₂)ₙ—, CHNR¹³R¹³, C═NR¹³, or NR¹³;
each R¹¹ is, independently, lower alkyl;
each R¹² is independently, —SO₂CH₃, —CO₂R¹³, —C(═O)NR¹³R¹³, —C(═O)R¹³, or —CH₂—(CHOH)ₙ—CH₂OH;
each R¹³ is, independently, hydrogen, R⁷, R¹⁰, —(CH₂)ₘ—NR¹³R¹³, 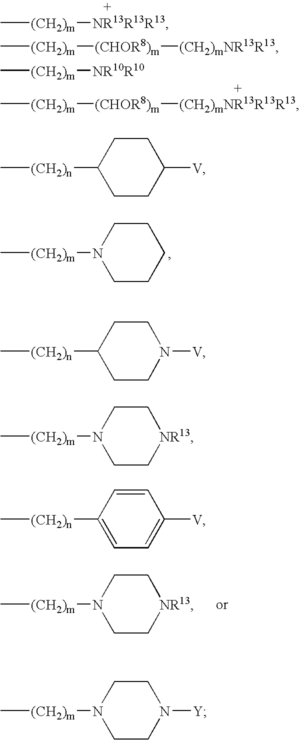
with the proviso that NR¹³R¹³ can be joined on itself to form a ring comprising one of the following: 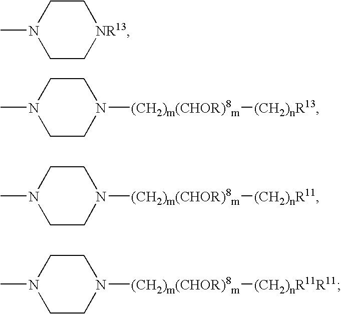
each Het is independently, —NR¹³, —S—, —SO—, or —SO₂—; —O—, —SO₂NR¹³—, —NHSO₂—, —NR¹³CO—, —CONR¹³—;
each g is, independently, an integer from 1 to 6;
each m is, independently, an integer from 1 to 7;
each n is, independently, an integer from 0 to 7;
each V is, independently, —(CH₂)ₘ—NR⁷R¹⁰, —(CH₂)ₘ—NR⁷R⁷, —(CH₂)ₘ— 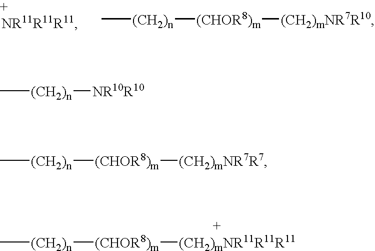
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3-with respect to each other the R⁸ groups may be joined to form a cyclic mono- or di-substituted 1,3-dioxane or 1,3-dioxolane;
or a pharmaceutically acceptable salt thereof, to an individual in need of prophylactic treatment against infection or disease from one or more airborne pathogens.

20. The prophylactic treatment method of claim 19 wherein the pathogen is Bacillus anthracis.

21. The prophylactic treatment method of claim 19 wherein the pathogen is Variola major.

22. The prophylactic treatment method of claim 19 wherein the pathogen is Yersinia pestis.

23. The prophylactic treatment method of claim 19 wherein the pathogen is Francisella tularensis.

24. The prophylactic treatment method of claim 19 wherein the pathogen is a gram negative bacteria.

25. The prophylactic treatment method of claim 24 wherein the gram negative bacteria is selected from the group consisting of Brucella species, Burkholderia pseudomallei, Burkholderia mallei, Coxiella burnetii and Rickettsia.

26. The prophylactic treatment method of claim 19 wherein the pathogen is an alphavirus, a flavivirus or a bunyavirus.

27. The prophylactic treatment method of claim 19 wherein the pathogen is ricin toxin from Ricinus communis, epsilon toxin of Clostridium perfringens or Staphylococcal enterotoxin B.

28. The prophylactic treatment method of claim 19 wherein the pathogen is Mycobacterium tuberculosis bacteria.

29. The prophylactic treatment method of claim 19 wherein the pathogen is an influenza virus, rhinovirus, adenovirus or respiratory syncytial virus.

30. The prophylactic treatment method of claim 19 wherein the pathogen is coronavirus.

31. The prophylactic treatment method of claim 19 wherein the sodium channel blocker or pharmaceutically acceptable salt thereof is administered in an aerosol suspension of respirable particles which the individual inhales.

32. The prophylactic treatment method of claim 19 wherein the sodium channel blocker or pharmaceutically acceptable salt thereof is administered for reducing the risk of infection from an airborne pathogen which can cause a disease in a human to the lungs of the human who may be at risk of infection from the airborne pathogen but is asymptomatic for the disease, wherein the effective amount of sodium channel blocker or a pharmaceutically acceptable salt is sufficient to reduce the risk of infection in the human.

33. The prophylactic treatment method of claim 19 wherein the sodium channel blocker or pharmaceutically acceptable salt thereof is administered post-exposure to the one or more airborne pathogens.

34. The prophylactic treatment method of claim 19 wherein the sodium channel blocker is selected from the group consisting of:

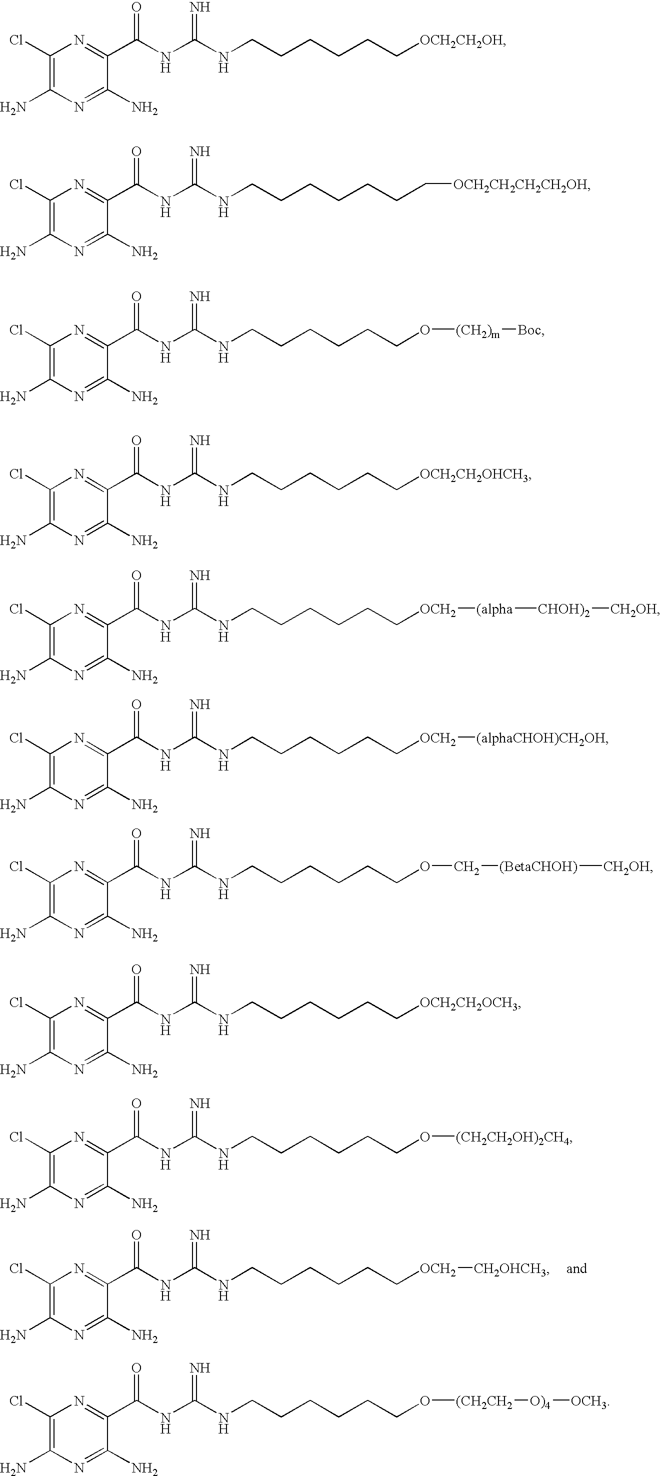

35. The prophylactic treatment method of claim 19 wherein the sodium channel blocker is selected from the group consisting of:

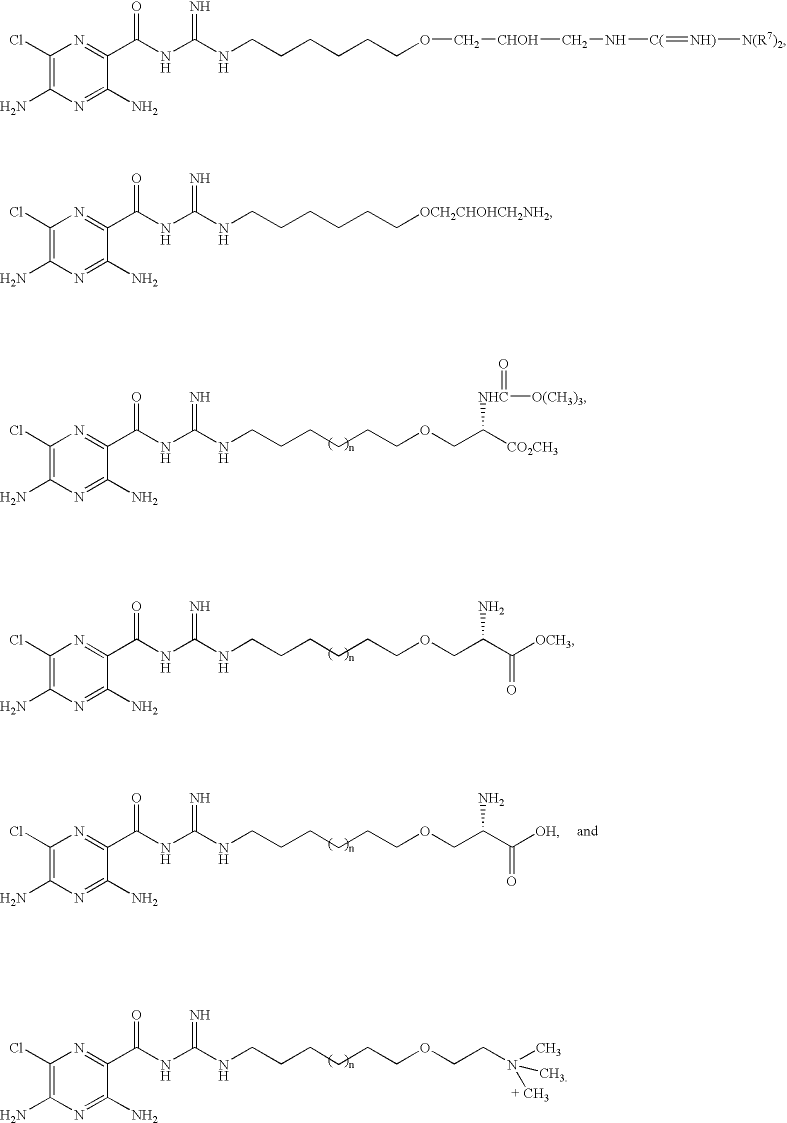

36. The prophylactic treatment method of claim 19 wherein the sodium channel blocker is selected from the group consisting of:

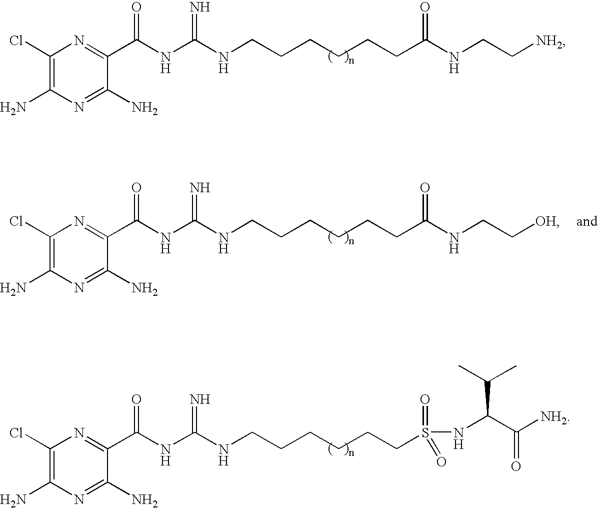

37. The prophylactic treatment method of claim 19 wherein the sodium channel blocker is selected from the group consisting of:

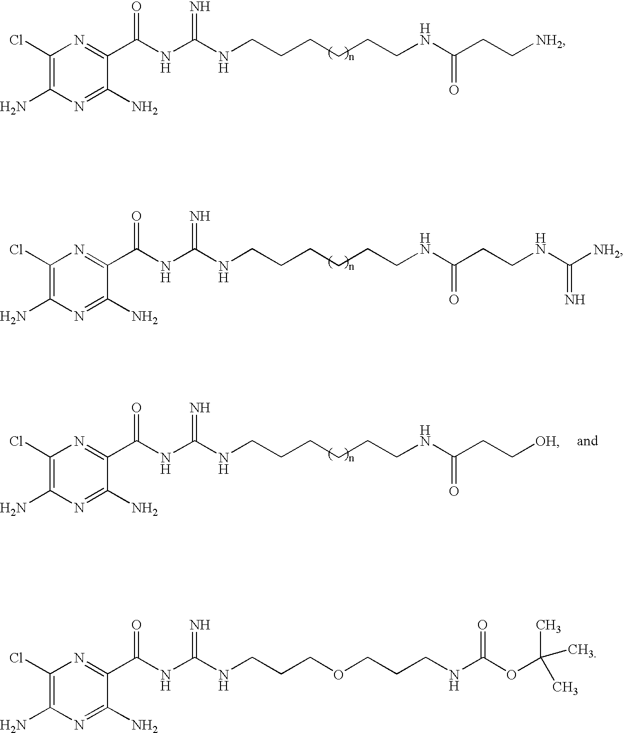

38. The prophylactic treatment method of claim 19 wherein the sodium channel blocker is selected from the group consisting of:

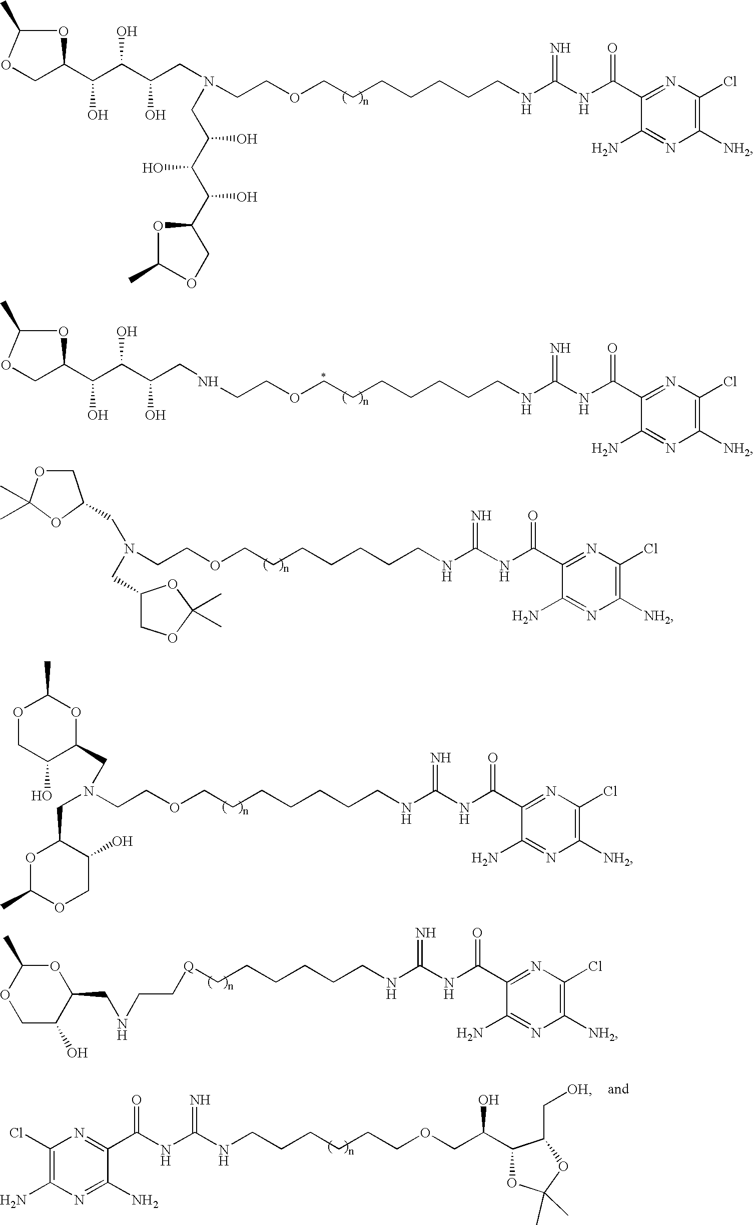

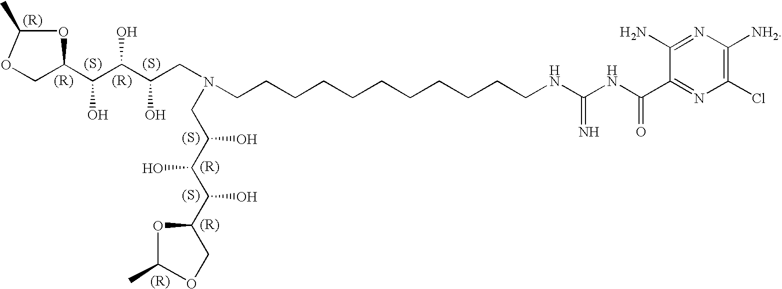

39. The prophylactic treatment method of claim 19 wherein the sodium channel blocker is selected from the group consisting of:

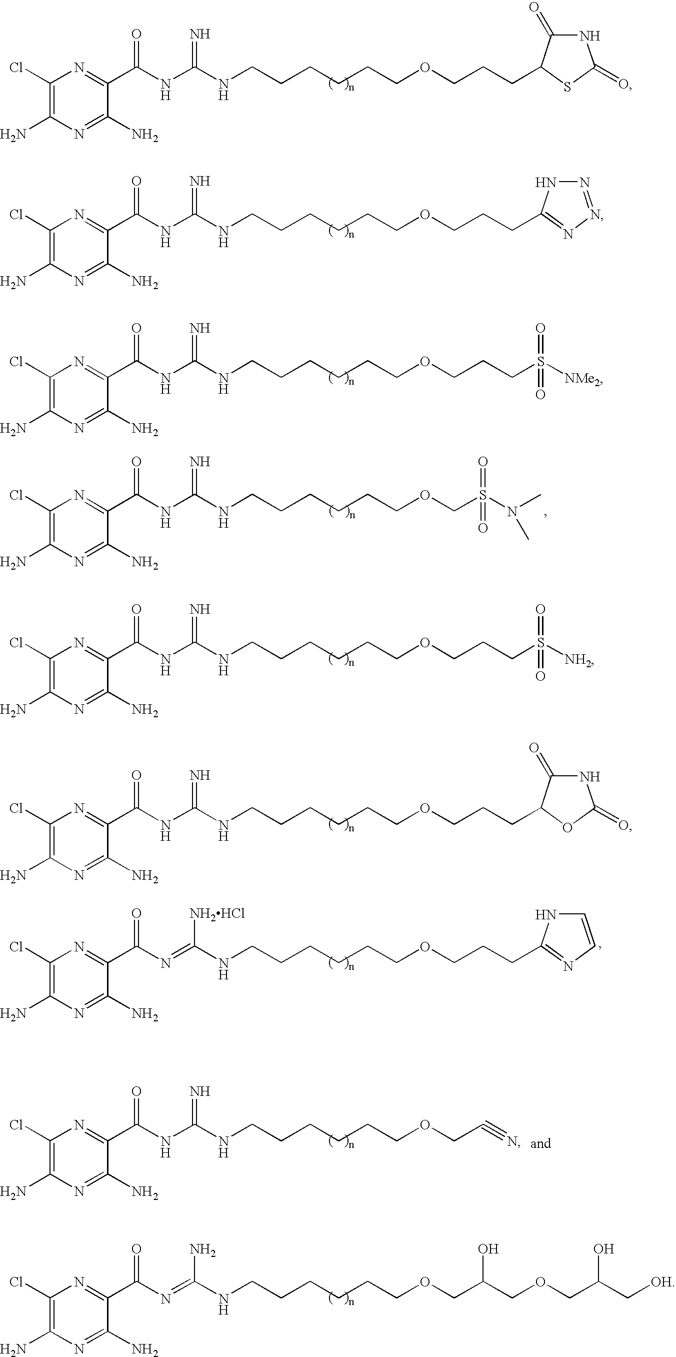

40. The prophylactic treatment method of claim 19 wherein the sodium channel blocker is selected from the group consisting of:

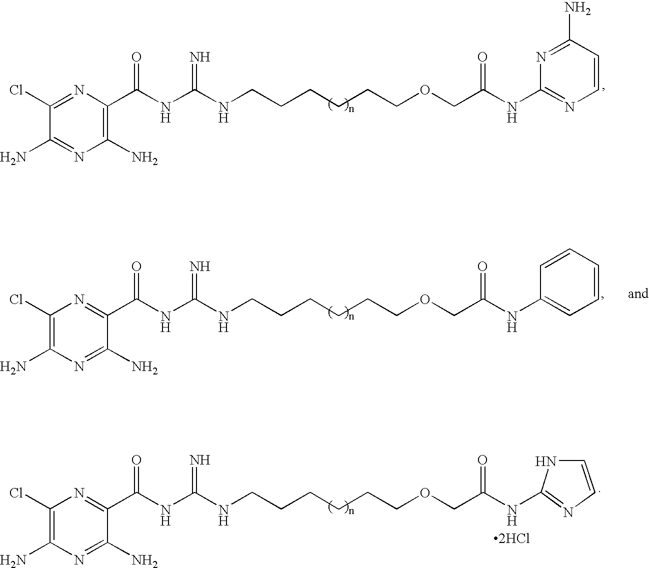

41. The prophylactic treatment method of claim 19 wherein the sodium channel blocker is selected from the group consisting of:

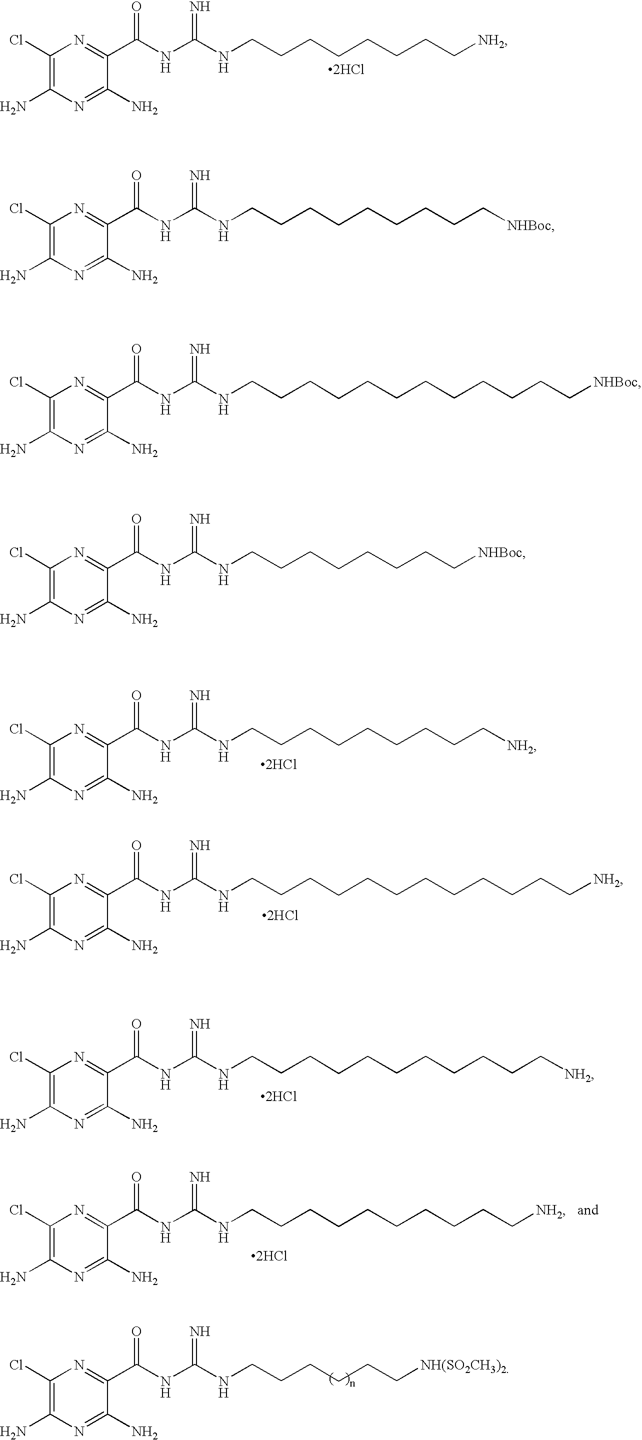

42. The prophylactic treatment method of claim 19 wherein the sodium channel blocker is selected from the group consisting of:

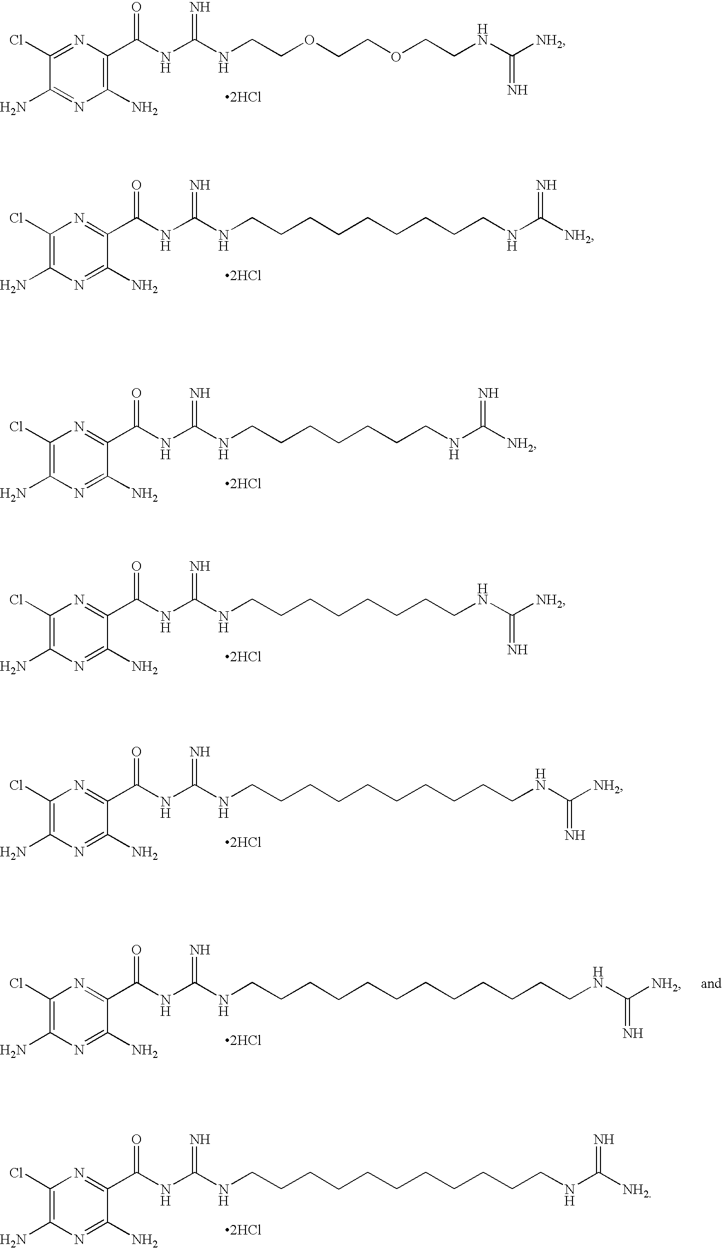

43. A prophylactic treatment method comprising: administering a prophylactically effective amount of a sodium channel blocker according to Formula III: 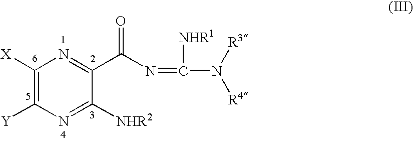
where
X is hydrogen, halogen, trifluoromethyl, lower alkyl, unsubstituted or substituted phenyl, lower alkyl-thio, phenyl-lower alkyl-thio, lower alkyl-sulfonyl, or phenyl-lower alkyl-sulfonyl;
Y is hydrogen, hydroxyl, mercapto, lower alkoxy, lower alkyl-thio, halogen, lower alkyl, unsubstituted or substituted mononuclear aryl, or —N(R²)₂;
R¹ is hydrogen or lower alkyl;
each R² is, independently, —R⁷, —(CH₂)ₘ—OR⁸, —(CH₂)ₘ—NR⁷R¹⁰, —(CH₂)ₙ(CHOR⁸)(CHOR⁸)ₙ—CH₂OR⁸, —(CH₂CH₂O)ₘ—R⁸, —(CH₂CH₂O)ₘ—CH₂CH₂NR⁷R¹⁰, —(CH₂)ₙ—C(═O)NR⁷R¹⁰, —(CH₂)ₙ-Z_{g}-R⁷, —(CH₂)ₘ—NR¹⁰—CH₂(CHOR⁸)(CHOR⁸)ₙ—CH₂OR⁸, —(CH₂)ₙ—CO₂R⁷, or 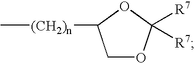
R^{3″} and R^{4″} are each, independently, hydrogen, a group represented by formula (A″), lower alkyl, hydroxy lower alkyl, phenyl, phenyl-lower alkyl, (halophenyl)-lower alkyl, lower-(alkylphenylalkyl), lower (alkoxyphenyl)-lower alkyl, naphthyl-lower alkyl, or pyridyl-lower alkyl, with the proviso that at least one of R^{3″} and R^{4″} is a group represented by formula (A″): 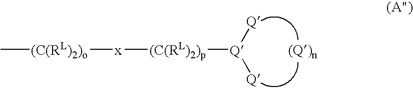
where
each R^{L} is, independently, —R⁷, —(CH₂)ₙ—OR⁸—O—(CH₂)ₘ—OR⁸, —(CH₂)ₙNR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘNR¹⁰CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 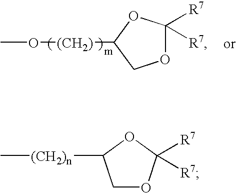
each o is, independently, an integer from 0 to 10;
each p is an integer from 0 to 10;
with the proviso that the sum of o and p in each contiguous chain is from 1 to 10;
each x is, independently, O, NR¹⁰, C(═O), CHOH, C(═N—R¹⁰), CHNR⁷R¹⁰, or represents a single bond;
each R^{5′} is, independently, independently, —O—(CH₂)ₘ—OR⁸, —(CH₂)ₙ—N⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂N⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH_{2OR}⁸, —O—(CH₂)ₘ—NR¹⁰—CH₂(CHOR⁸)(CHOR⁸)ₙ—CH₂OR⁸, —(CH₂)ₙ—CO₂R⁷, —O(CH₂)ₘ—CO₂R⁷, —OSO₃H, —O-glucuronide, —O-glucose, 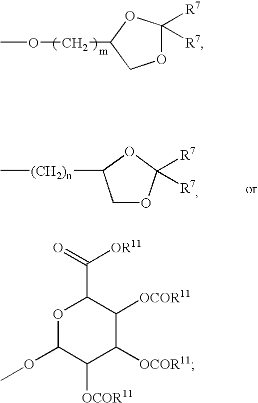
each R^{5′} is also, independently, —(CH₂)ₙ—NR¹²R¹², —O—(CH₂)ₘ—NR¹²R¹², —O—(CH₂)ₙ—NR¹²R¹², —O—(CH₂)ₘ-(Z)_{g}R¹², —(CH₂)ₙNR¹¹R¹¹, —O—(CH₂)ₘNR¹¹R¹¹, —(CH₂)ₙ—N^{⊕}—(R¹¹)₃, —O—(CH₂)ₘN^{⊕}—(R¹¹)₃, —(CH₂)ₙ-(Z)_{g}-(CH₂)ₘ—NR¹⁰R¹⁰, —O—(CH₂)ₘ-(Z)_{g}-(CH₂)ₘ—NR¹⁰R¹⁰ , —(CH₂CH₂O)ₘ—CH₂CH₂NR¹²R¹², —O—(CH₂CH₂O)ₘCH₂CH₂NR¹²R¹², —(CH₂)ₙ—(C═O)NR¹²R¹², —O—(CH₂)ₘ—(C═O)NR¹²R¹², —O—(CH₂)ₘ—(CHOR⁸)ₘCH₂NR¹⁰-(Z)_{g}-R¹⁰, —(CH₂)ₙ-(CHOR⁸)ₘCH₂—NR¹⁰-(Z)_{g}-R¹⁰, —(CH₂)ₙNR¹⁰—O(CH₂)ₘ(CHOR⁸)ₙCH₂NR¹⁰-(Z)_{g}-R¹⁰, —O(CH₂)ₘ—NR¹⁰—(CH₂)ₘ—(CHOR⁸)ₙCH₂NR¹⁰-(Z)_{g}-R¹⁰, -(Het)-(CH₂)ₘ—OR⁸, -(Het)-(CH₂)ₘ—NR⁷R¹⁰, -(Het)-(CH₂)ₘ(CHOR⁸)(CHOR⁸)ₙ—CH₂OR⁸, -(Het)-(CH₂CH₂O)ₘ—R⁸, -(Het)-(CH₂CH₂O)ₘ—CH₂CH₂NR⁷R¹⁰, -(Het)-(CH₂)ₘ—C(=O)NR⁷R¹⁰, -(Het)-(CH₂)ₘ-(Z)_{g}-R⁷, -(Het)-(CH₂)ₘ—NR¹⁰—CH₂(CHOR⁸)(CHOR⁸)ₙ—CH₂OR⁸, -(Het)-(CH₂)ₘ—CO₂R⁷, -(Het)-(CH₂)ₘ—NR¹²R¹², -(Het)-(CH₂)ₙ—NR¹²R¹², -(Het)-(CH₂)ₘ-(Z)_{g}R¹², -(Het)-(CH₂)ₘNR¹¹R¹¹, -(Het)-(CH₂)ₘ—N^{⊕}-(R¹¹)₃, -(Het)-(CH₂)ₘ-(Z)_{g}-(CH₂)ₘ—NR¹⁰R¹⁰, -(Het)-(CH₂CH₂O)ₘ—CH₂CH₂NR¹²R¹², -(Het)-(CH₂)ₘ—(C═O)NR¹²R¹², -(Het)-(CH₂)ₘ—(CHOR⁸)ₘCH₂NR¹⁰-(Z)_{g}-R¹⁰, -(Het)-(CH₂)ₘ—NR¹⁰—(CH₂)ₘ—(CHOR⁸)ₙCH₂NR¹⁰-(Z)_{g}-R¹⁰,
wherein when two —CH₂OR⁸ groups are located 1,2- or 1,3-with respect to each other the R⁸ groups may be joined to form a cyclic mono- or di-substituted 1,3-dioxane or 1,3-dioxolane,
—(CH₂)ₙ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—O—(CH₂)ₘ(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane,
—(CH₂)ₙ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane, or
—O—(CH₂)ₘ—NR¹⁰—CH₂(CHOR⁸)(CHOR⁸)ₙ—CH₂OR⁸, with the proviso that at least two —CH₂OR⁸ are located adjacent to each other and the R⁸ groups are joined to form a cyclic mono- or di-substituted 1,3-dioxane or 1,3-dioxolane;
wherein each R^{5′} is also, independently,
Link —(CH₂)ₙ—CAP, Link —(CH₂)ₙ(CHOR⁸)(CHOR⁸)ₙ—CAP, Link —(CH₂CH₂O)ₘ—CH₂—CAP, Link —(CH₂CH₂O)ₘ—CH₂CH₂—CAP, Link —(CH₂)ₙ-(Z)_{g}-CAP, Link —(CH2)ₙ(Z)_{g}-(CH₂)ₘ—CAP, Link —(CH₂)ₙ—NR¹³—CH₂(CHOR⁸)(CHOR⁸)ₙ—CAP, Link —(CH₂)ₙ—(CHOR⁸)ₘCH₂—NR¹³-(Z)_{g}-CAP, Link —(CH₂)ₙNR¹³—(CH₂)ₘ(CHOR⁸)ₙCH₂NR¹³-(Z)_{g}-CAP, Link —(CH₂)ₘ-(Z)_{g}-(CH₂)ₘ—CAP, Link NH—C(═O)—NH—(CH₂)ₘ—CAP, Link —(CH₂)ₘ—C(═O)NR¹³—(CH₂)ₘ—C(═O)NR¹⁰R¹⁰, Link —(CH₂)ₘ—C(═O)NR¹³—(CH₂)ₘ—CAP, Link —(CH₂)ₘ—C(═O)NR¹¹R¹¹, Link —(CH₂)ₘ—C(═O)NR¹²R¹², Link —(CH₂)ₙ-(Z)_{g}-(CH₂)ₘ-(Z)_{g}-CAP, Link -Z_{g}-(CH₂)ₘ-Het-(CH₂)ₘ—CAP;
wherein Link is, independently,
—O—, (CH₂)ₙ—, —O(CH₂)ₘ—, —NR¹³—C(═O)—NR¹³, —NR¹³—C(═O)—(CH₂)ₘ—, —C(═O)NR¹³—(CH₂)ₘ, —(CH₂)ₙ-Z_{g}-(CH₂)ₙ, —S—, —SO—, —SO₂—, SO₂NR⁷—, SO₂NR¹⁰—, -Het-;
wherein each CAP is, independently,
thiazolidinedione, oxazolidinedione, heteroaryl-C(═O)N R¹³R¹³, heteroaryl-CAP, —CN, —O—C(═S)NR¹³R¹³, —Z_{g}R¹³, —CR¹⁰(Z_{g}R¹³)(Z_{g}R¹³), —C(═O)OAr , —C(═O)N R¹³Ar, imidazoline, tetrazole, tetrazole amide, —SO₂NHR¹³, —SO₂NH—C(R¹³R¹³)-(Z)_{g}R¹³, cyclic sugars and oligosaccharides, including cyclic amino sugars and oligosaccharides, 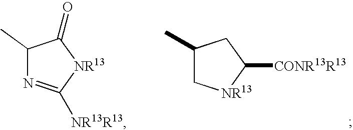
wherein Ar is, independently, phenyl; substituted phenyl, wherein said substituent is 1-3 groups selected, independently, from OH, OCH₃, NR¹³R¹³, Cl, F, CH₃; heteroaryl, tinazine, furyl, furfuryl-, thienyl, tetrazole, thiazolidinedione or imidazoyl ( 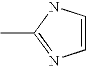
);
wherein heteroaryl is selected from one of the following heteroaromatic systems:
Pyrrole, Furan, Thiophene, Pyridine, Quinoline, Indole, Adenine, Pyrazole, Imidazole, Thiazole, Isoxazole, Indole, Benzimidazole, Purine, Quinoline, Isoquinoline, Pyridazine, Pyrimidine, Pyrazine, 1,2,3-Triazine, 1,2,4-Triazine, 1,3,5-Triazine, Cinnoline, Phthalazine, Quinazoline, Quinoxaline and Pterdine;
wherein when two —CH₂OR⁸ groups are located 1,2- or 1,3-with respect to each other the R⁸ groups may be joined to form a cyclic mono- or di-substituted 1,3-dioxane or 1,3-dioxolane;
each R^{6′} is, independently, —R^{5′}, —R⁷, —OR⁸, —N(R⁷)₂, —(CH₂)ₘ—OR⁸, —O—(CH₂)ₘ—OR⁸, —(CH₂)ₙ—NR⁷R¹⁰, —O—(CH₂)ₘ—NR⁷R¹⁰, —(CH₂)ₙ(CHOR⁸)(CHOR⁸)ₙ—CH₂OR⁸, —O—(CH₂)ₘ(CHOR⁸)(CHOR⁸)ₙ—CH₂OR⁸, —(CH₂CH₂O)ₘ—R⁸, —O—(CH₂CH₂O)ₘ—R⁸, —(CH₂CH₂O)ₘ—CH₂CH₂NR⁷R¹⁰, —O—(CH₂CH₂O)ₘ—CH₂CH₂NR⁷R¹⁰, —(CH₂)ₙ—C(═O)NR⁷R¹⁰, —O—(CH₂)ₘ—C(═O)NR⁷R¹⁰, —(CH₂)ₙ-(Z)_{g}-R⁷, —O—(CH₂)ₘ-(Z)_{g}-R⁷, —(CH₂)ₙ—NR¹⁰—CH₂(CHOR⁸)(CHOR⁸)ₙ—CH₂OR⁸, —O—(CH₂)ₘ—NR¹⁰—CH₂(CHOR⁸)(CHOR⁸)ₙ—CH₂OR⁸, —(CH₂)ₙ—CO₂R⁷, —O—(CH₂)ₘ—CO₂R⁷, —OSO₃H, —O-glucuronide, —O-glucose, 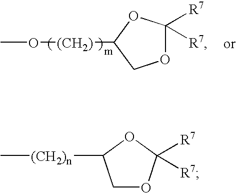
wherein when two —CH₂OR⁸ groups are located 1,2- or 1,3-with respect to each other the R⁸ groups may be joined to form a cyclic mono- or di-substituted 1,3-dioxane or 1,3-dioxolane;
each R⁷ is, independently, hydrogen lower alkyl, phenyl, substituted phenyl or —CH₂(CHOR)⁸ₘ—R¹⁰;
each R⁸ is, independently, hydrogen, lower alkyl, —C(═O)—R¹¹, glucuronide, 2-tetrahydropyranyl, or 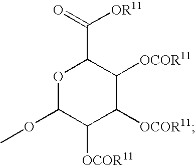
each R⁹ is, independently, —CO₂R¹³, —CON(R¹³)₂, —SO₂CH₂R¹³, or —C(═O)R¹³;
each R¹⁰ is, independently, —H, —SO₂CH₃, —CO₂R¹³, —C(═O)NR¹³R¹³, —C(═O)R¹³, or —(CH₂)ₘ—(CHOH)ₙ—CH₂OH;
each Z is, independently, CHOH, C(═O), —(CH₂)ₙ—, CHNR¹³R¹³, C═NR¹³, or NR¹³;
each R¹¹ is, independently, lower alkyl;
each R¹² is independently, —SO₂CH₃, —CO₂R¹³, —C(═O)NR¹³R¹³, —C(═O)R¹³, or —CH₂—(CHOH)ₙ—CH₂OH;
each R¹³ is, independently, hydrogen, R⁷, R¹⁰, —( CH₂)ₘ—N¹³R¹³, 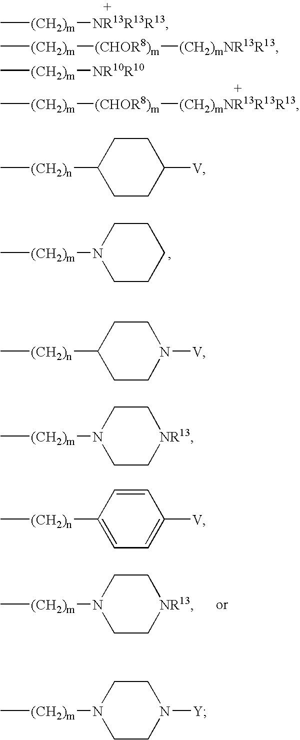
with the proviso that NR¹³R¹³ can be joined on itself to form a ring comprising one of the following: 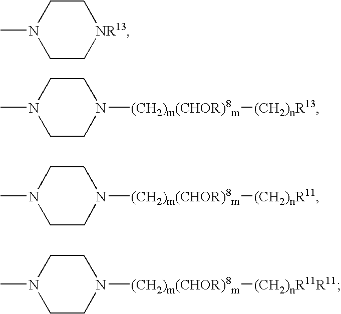
each Het is independently, —NR¹³, —S—, —SO—, or —SO₂—; —O—, —SO₂NR¹³—, —NHSO₂—, —NR¹³CO—, —CONR¹³—;
each g is, independently, an integer from 1 to 6;
each m is, independently, an integer from 1 to 7;
each n is, independently, an integer from 0 to 7;
each Q′ is, independently, —CR^{6′}R^{5′}, —CR^{6′}R^{6′}, N, —NR¹³, —SO—, or —SO₂—;
wherein at most three Q′ in a ring contain a heteroatom and at least one Q′ must be —CR^{5′}R^{6′} or NR^{5′};
each V is, independently, —(CH₂)ₘ—NR⁷R¹⁰, —(CH₂)ₘ—NR⁷R⁷, —(CH₂)ₘ— 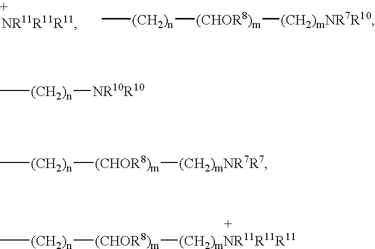
with the proviso that when V is attached directly to a nitrogen atom, then V can also be, independently, R⁷, R¹⁰, or (R¹¹)₂;
wherein for any of the above compounds when two —CH₂OR⁸ groups are located 1,2- or 1,3-with respect to each other the R⁸ groups may be joined to form a cyclic mono- or di-substituted 1,3-dioxane or 1,3-dioxolane;
or a pharmaceutically acceptable salt thereof, to an individual in need of prophylactic treatment against infection or disease from one or more airborne pathogens.

44. The prophylactic treatment method of claim 43 wherein the pathogen is Bacillus anthracis.

45. The prophylactic treatment method of claim 43 wherein the pathogen is Variola major.

46. The prophylactic treatment method of claim 43 wherein the pathogen is Yersinia pestis.

47. The prophylactic treatment method of claim 43 wherein the pathogen is Francisella tularensis.

48. The prophylactic treatment method of claim 43 wherein the pathogen is a gram negative bacteria.

49. The prophylactic treatment method of claim 48 wherein the gram negative bacteria is selected from the group consisting of Brucella species, Burkholderia pseudomallei, Burkholderia mallei, Coxiella burnetii and Rickettsia.

50. The prophylactic treatment method of claim 43 wherein the pathogen is an alphavirus, a flavivirus or a bunyavirus.

51. The prophylactic treatment method of claim 43 wherein the pathogen is ricin toxin from Ricinus communis, epsilon toxin of Clostridium perfringens or Staphylococcal enterotoxin B.

52. The prophylactic treatment method of claim 43 wherein the pathogen is Mycobacterium tuberculosis bacteria.

53. The prophylactic treatment method of claim 43 wherein the pathogen is an influenza virus, rhinovirus, adenovirus or respiratory syncytial virus.

54. The prophylactic treatment method of claim 43 wherein the pathogen is coronavirus.

55. The prophylactic treatment method of claim 43 wherein the sodium channel blocker or pharmaceutically acceptable salt thereof is administered in an aerosol suspension of respirable particles which the individual inhales.

56. The prophylactic treatment method of claim 43 wherein the sodium channel blocker or pharmaceutically acceptable salt thereof is administered for reducing the risk of infection from an airborne pathogen which can cause a disease in a human to the lungs of the human who may be at risk of infection from the airborne pathogen but is asymptomatic for the disease, wherein the effective amount of sodium channel blocker or a pharmaceutically acceptable salt is sufficient to reduce the risk of infection in the human.

57. The prophylactic treatment method of claim 43 wherein the sodium channel blocker or pharmaceutically acceptable salt thereof is administered post-exposure to the one or more airborne pathogens.

58. The prophylactic treatment method of claim 43 wherein the sodium channel blocker is selected from the group consisting of:

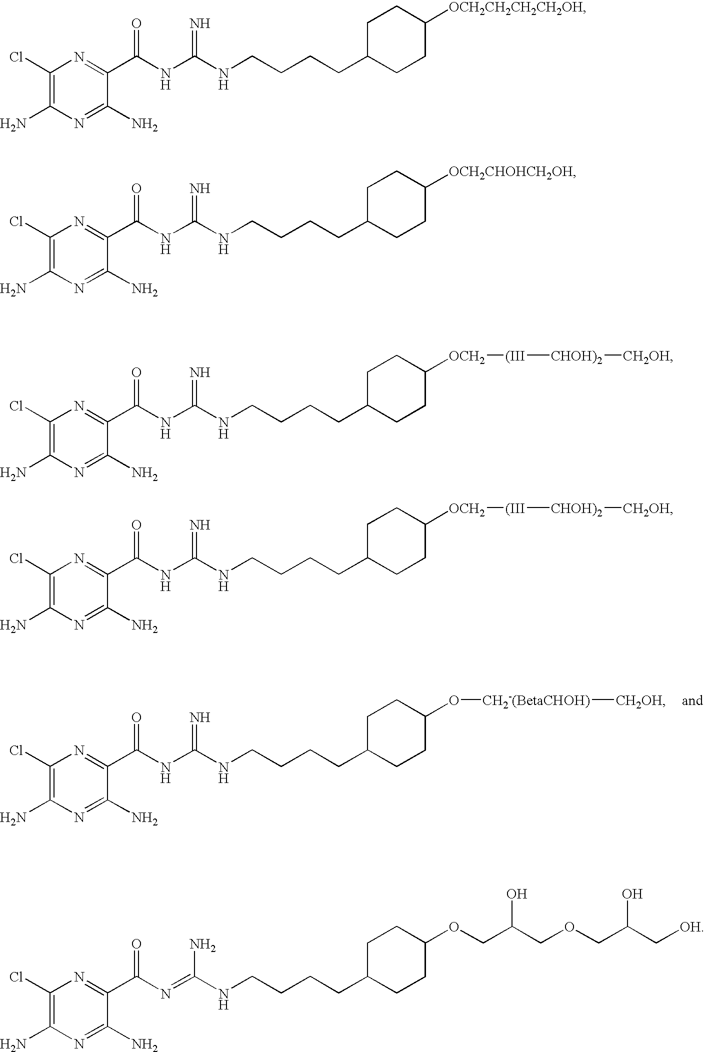

59. The prophylactic treatment method of claim 43 wherein the sodium channel blocker is selected from the group consisting of:

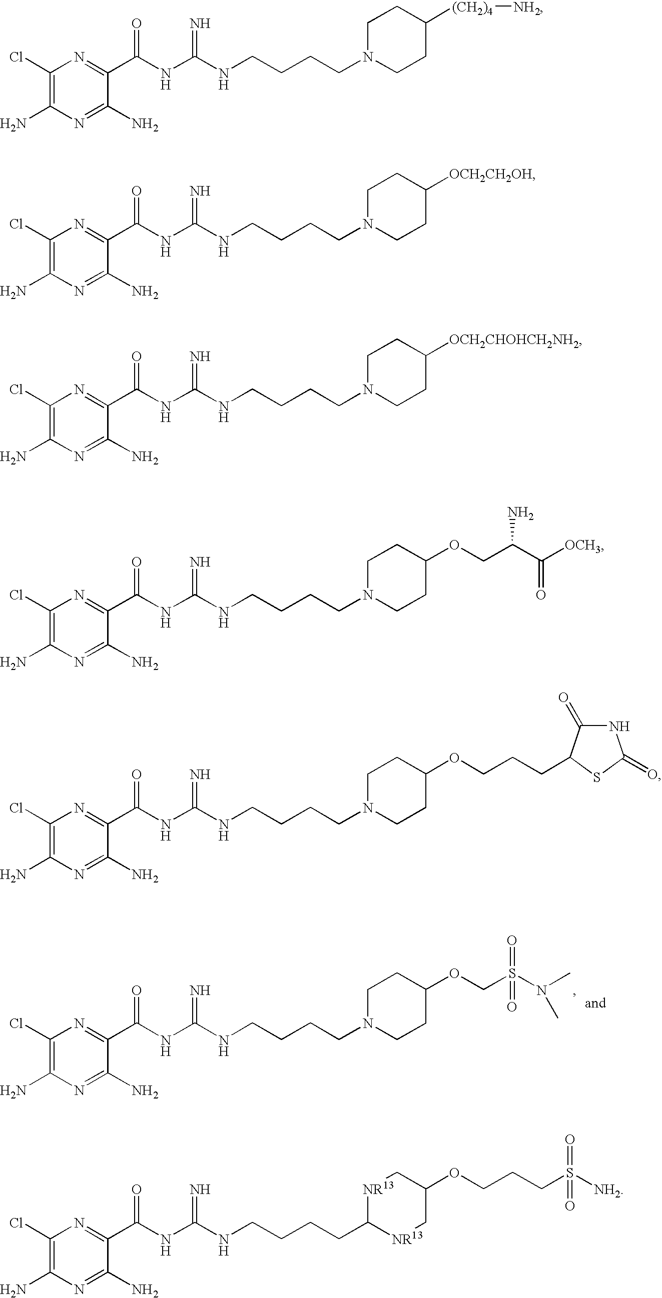

60. The prophylactic treatment method of claim 43 wherein the sodium channel blocker is selected from the group consisting of:

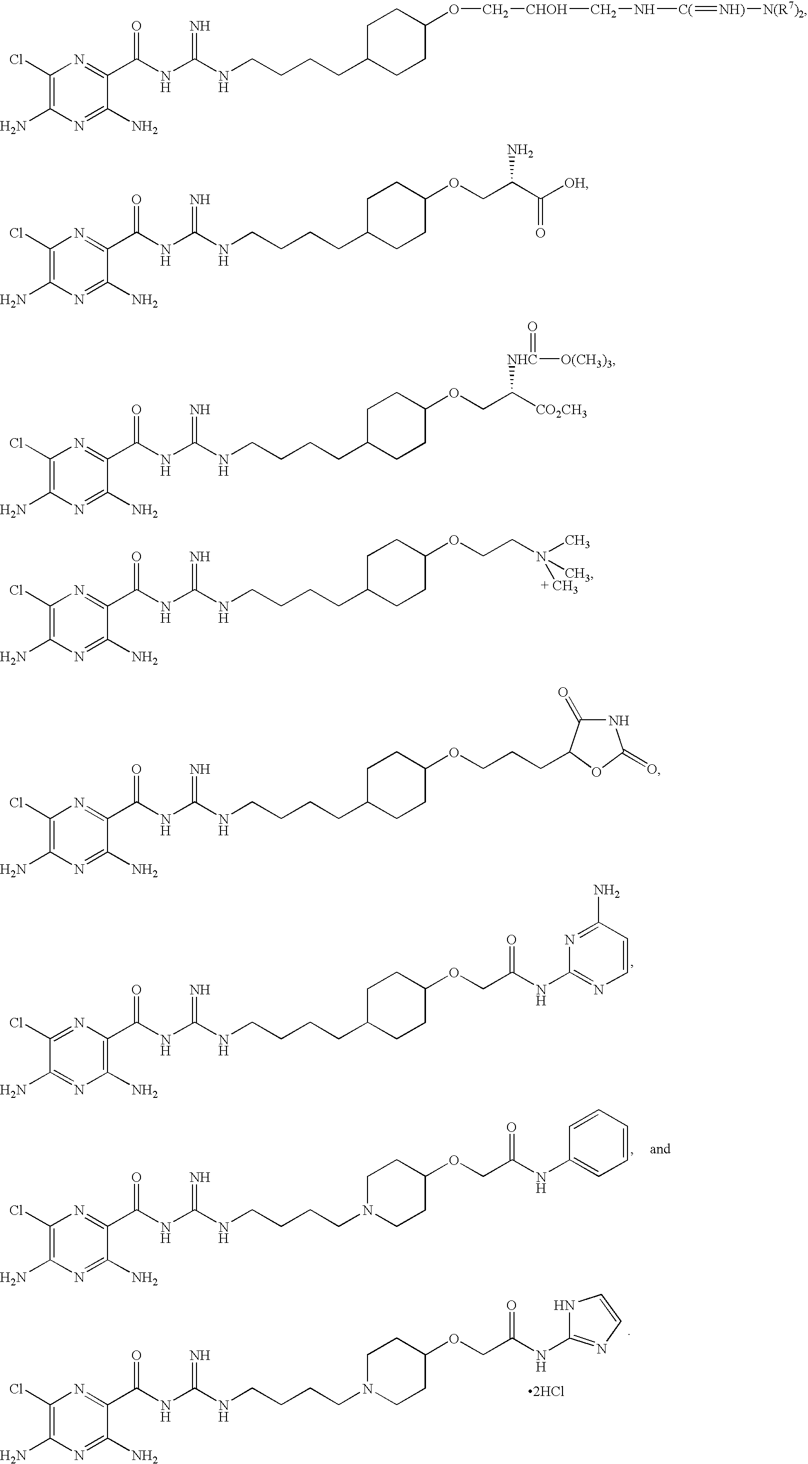

61. The prophylactic treatment method of claim 43 wherein the sodium channel blocker is selected from the group consisting of:

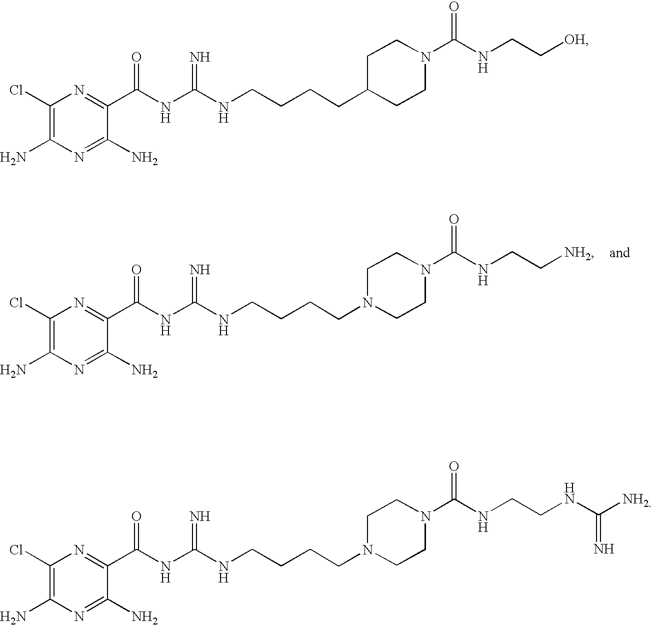

62. The prophylactic treatment method of claim 43 wherein the sodium channel blocker is selected from the group consisting of:

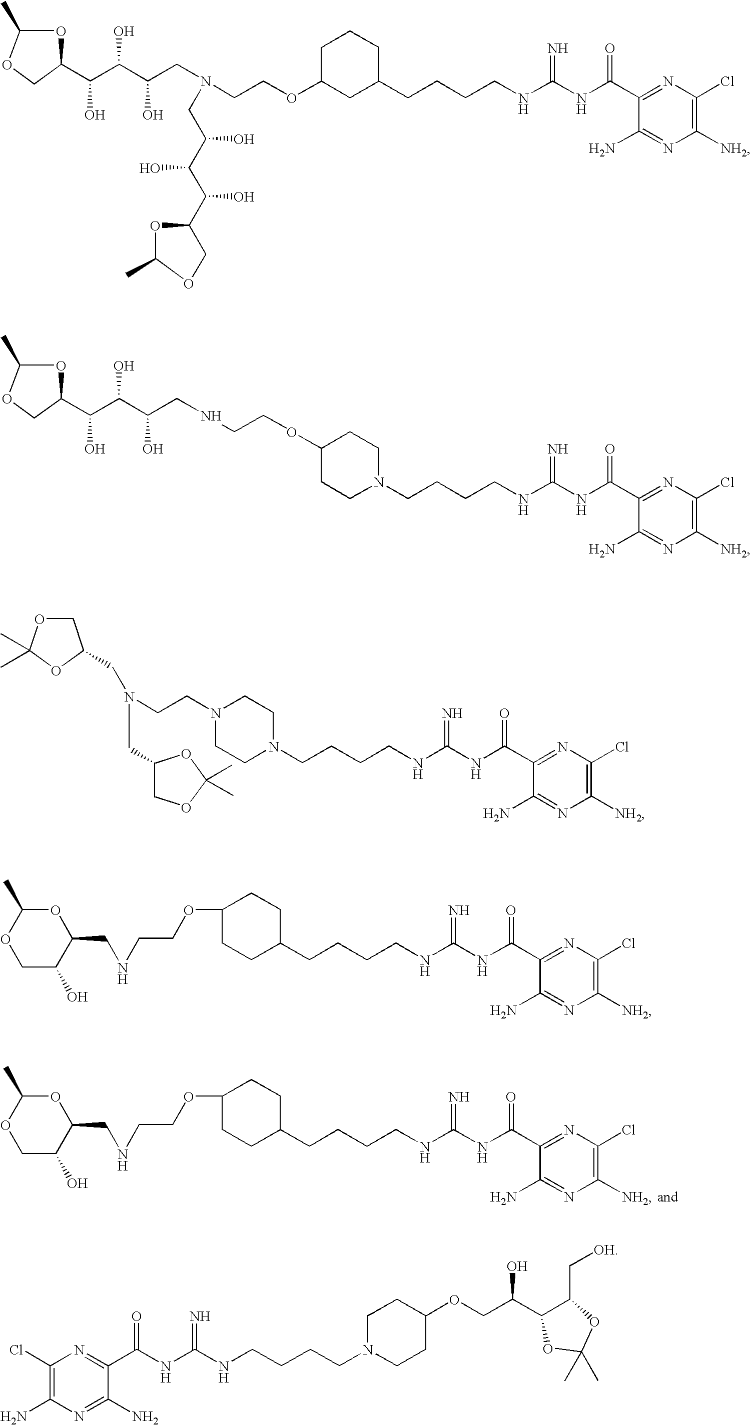

63. The prophylactic treatment method of claim 43 wherein the sodium channel blocker is selected from the group consisting of:

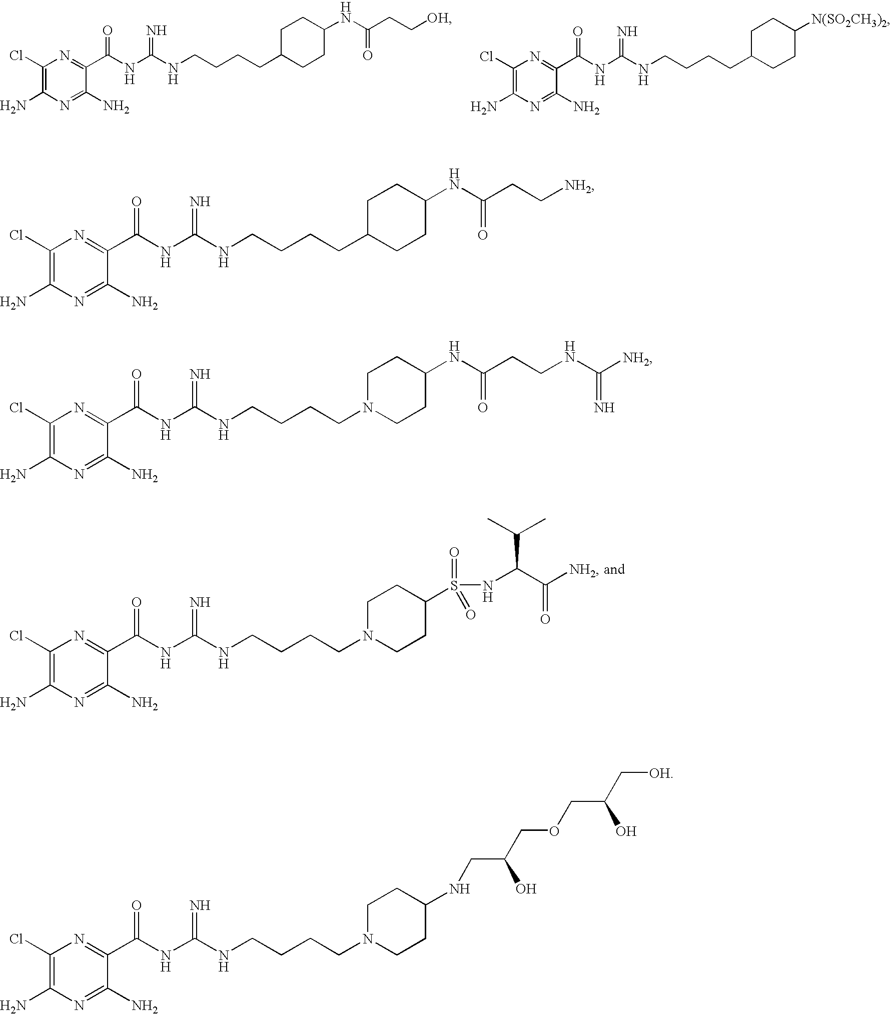

64. The prophylactic treatment method of claim 43 wherein the sodium channel blocker is selected from the group consisting of:

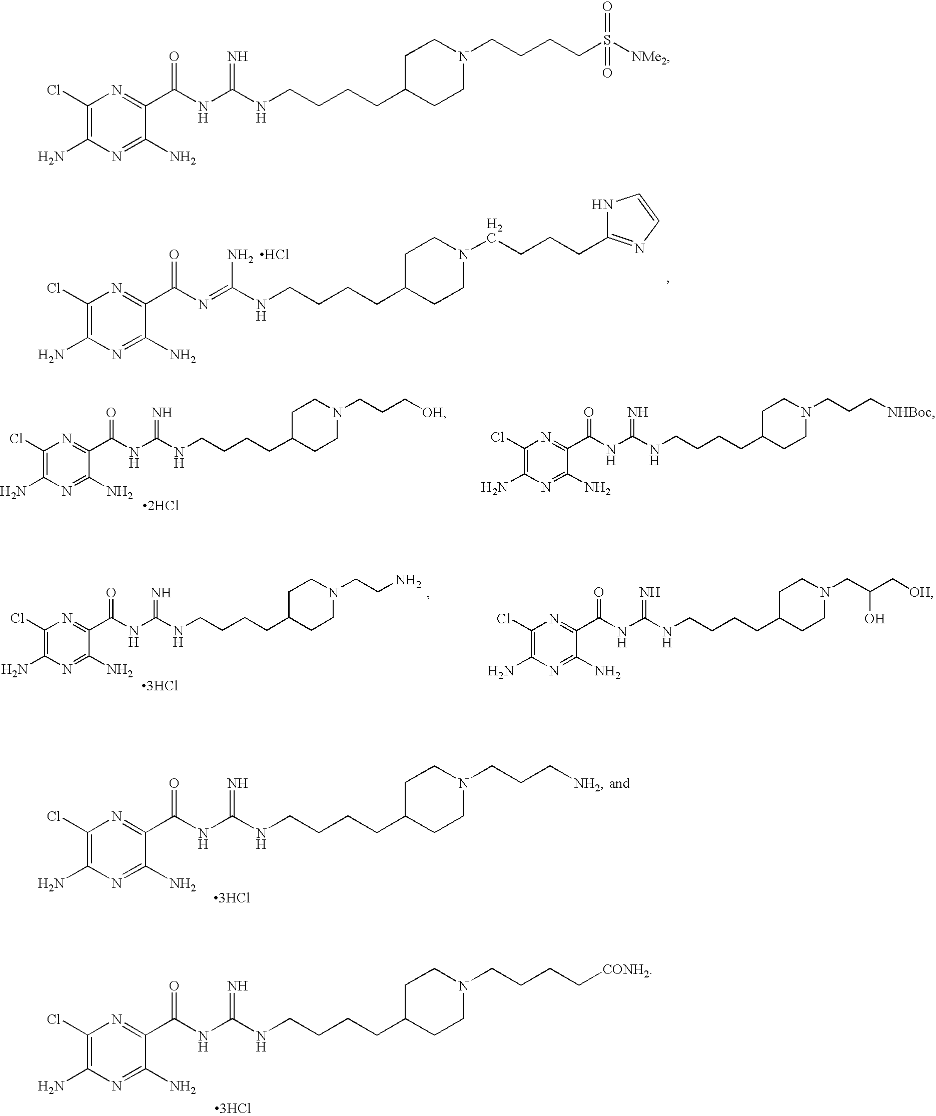

65. The prophylactic treatment method of claim 43 wherein the sodium channel blocker is selected from the group consisting of:

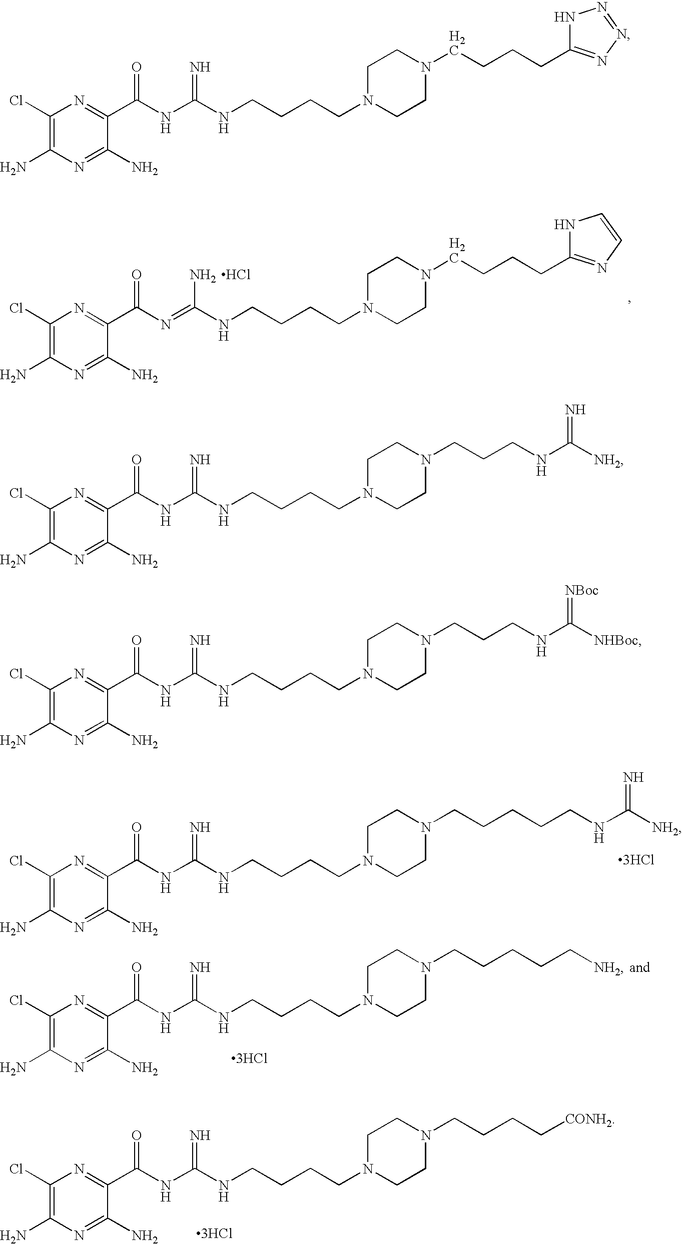

66. The prophylactic treatment method of claim 43 wherein the sodium channel blocker is selected from the group consisting of:

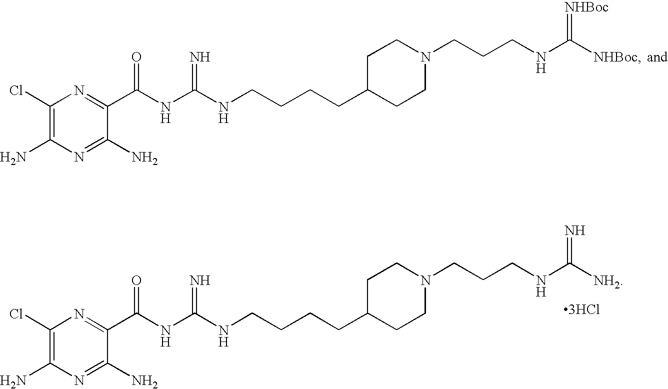

67. The prophylactic treatment method of claim 43 wherein the sodium channel blocker is selected from the group consisting of: [chemical structure]
